(19) United States
(12) Patent Application Publication
(10) Pub. No.: US 2006/0116390 A1
(43) Pub. Date: Jun. 1, 2006

(54) Hepatitis c virus inhibitors

(76) Inventors: Jae-Hong Lim, Daejeon (KR); Joo-Yong Yoon, Daejeon (KR); Jeong-Uk Song, Daejeon (KR); Lee-Taek Sung, Daejeon (KR); Sung-Pil Choi, Daejeon (KR); Ho-Young Song, Daejeon (KR); Jong-Yup Kim, Daejeon (KR); Yong-Zu Kim, Daejeon (KR); Young-Gyu Cho, Daejeon (KR); Chang-Myung Kim, Daejeon (KR); Won-Sup Kim, Daejeon (KR); Seung-Wan Kang, Daejeon (KR); Ji-Hyun Park, Daejeon (KR)

Correspondence Address: BIRCH STEWART KOLASCH & BIRCH, PO BOX 747, FALLS CHURCH, VA, 22040-0747, US

(21) Appl. No.: 10/559,746

(22) PCT Filed: Jun. 9, 2004
(86) PCT No.: PCT/KR04/01370

§ 371 Date: Dec. 7, 2005

(30) Foreign Application Priority Data
Jun. 13, 2003 (KR) 10-2003-0038246

(51) Int. Cl. A61K 31/513; C07D 239/22

(52) U.S. Cl. 514/269; 544/314

(57) ABSTRACT

The present invention relates to new compounds useful for the treatment or prevention of hepatitis C, process for preparing them, and a composition for the treatment or prevention of hepatitis C comprising the compounds as an active ingredient.

### TECHNICAL FIELD

The present invention relates to new compounds useful for the treatment or prevention of hepatitis C, a process for preparing them, and a composition for the treatment or prevention of hepatitis C comprising the compounds as an active ingredient. The composition of the present invention is particularly effective for inhibiting activity of the hepatitis C virus (‘HCV,’ below), and so can be advantageously used as a therapeutic agent to the infection with hepatitis C virus or other viruses.

### BACKGROUND ART

HCV belongs to flaviviridae, and had been known as the major pathogen of Non-A and Non-B hepatitis which are infected by blood transfusion before a part of its gene was cloned for the first time. More than 1% of the total population in the world are presumed to have antibody against HCV by the HCV infection [Review; Purcell, R. H., ‘Hepatitis C virus: Historical perspective and current concepts’ FEMS Microbiology Reviews’ 14, pp 181-192 (1994); Van der Poel, C. L., ‘Hepatitis C Virus. Epidemiology, Transmission and Prevention in Hepatitis C Virus. Current Studies in Hematology and Blood Transfusion, H. W. Reesink, Ed., (Basel: Karger), pp 137-163 (1994)], and particularly, four (4) million persons in the USA are presumed to be infected thereby [Alter, M. J. and Mast, E. E., ‘The Epidemiology of Viral Hepatitis in the United States, Gastroenterol. Clin. North Am. 23, pp 437-455 (1994)].

When human beings are first exposed to HCV, only about 20% of the infected persons suffer from acute hepatitis, and the others do not show severe symptoms. In most cases, however, the virus induces chronic hepatitis lasting several decades [Iwarson, S., ‘The Natural Course of Chronic Hepatitis’ FEMS Microbiology Reviews 14, pp 201-204 (1994)]. Generally (60% or more), the chronic hepatitis causes liver infection to be recurrent or to get gradually worse, and is frequently developed into more serious diseases such as hepatocirrhosis, hepatocellular carcinoma, etc. [Kew, M C., ‘Hepatitis C and Hepatocellular Carcinona’, FEMS Microbiology Reviews, 14, pp 211-220 (1994); Saito, I., ‘Hepatitis C Virus Infection is Associated with the Development of Hepatocellular Carcinoma’ Proc. Natl. Acd. Sci. USA 87, p 6547-6549 (1990)]. A lot of HCV infection by blood transfusion Ray be prevented by the development of medicines for dignosis, but new infections are still found out by some unconfirmed pathways. Unfortunately, there is no broadly effective therapeutic method that attenuates progressing chronic HCV.

Further, under the current knowledge for HCV, no satisfactory HCV inhibitor or therapeutic agent has been developed. The only earlier therapeutic method for HCV-related diseases is interferon therapy or a combination therapy of interferon and ribavirin [Kallinowski, B et al., Zeitschrift fur Gastroenterologie 39, 199-206(2001)]. However, interferon shows considerable side effects (Janssen et al., 1994; Renault and Hoofnagle, 1989) [Janssen et al., Suicide Associated with Alfa-Interferon Therapy for Chronic Viral Hepatitis’, J. Hepatol., 21, pp 241-243 (1994); Renault, P. F. and Hoofnagle, J. H., Side effects of alphainterferon. Seminars in Liver Disease 9, 273-277. (1989)], and has a long term therapeutic effect only for some cases (˜25%) [Weiland, O., ‘Interferon Therapy in Chronic Hepatitis C Virus Infection’, FEMS Microbiol. Rev., 14, pp 279-288 (1994)]. Thus, a more potent anti-HCV therapeutic agent is needed.

Under such circumstances as explained above, the present inventors have extensively studied to develop potent HCV inhibitors which can be used more effectively for the treatment or prevention of hepatitis C. As a result, the present inventors have discovered that a group of new compounds as represented by the following formula (1) can have such desired effect, and then completed the present invention.

### DISCLOSURE

Therefore, the present invention is to provide a compound of the following formula (1):

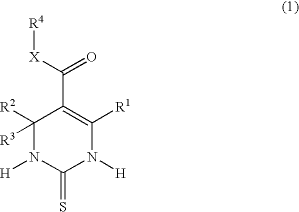

in which

X is O or S,

R¹ is hydrogen,

alkyl unsubstituted or substituted by 1 to 3 substituents selected from a group consisting of halogen; alkoxy; alkoxycarbonyl; hydroxy; carboxy; and

aryl,

alkoxy,

alkoxycarbonyl unsubstituted or substituted by aryl, or

aryl,

R² and R³ each are

hydrogen,

alkyl substituted by 1 to 3 substituents selected from a group consisting of halogen; hydroxy; alkoxy; alkylsulfonyl; and aralkyloxy,

alkoxycarbonyl,

alkylsulfonylakyl,

aryl, or

heteroaryl, or

R² and R³ together form cycloalkyl with the carbon atom to which they are attached, or

when one of R² and R³ is hydrogen, the other is a structure selected from the following:

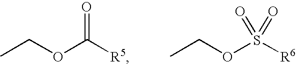

where

R⁵ is alkyl unsubstituted or substituted by amino,

alkylamino unsubstituted or substituted by alkylcarbonylamino; alkylcarbonyloxy; or dialkylamino,

alkoxy unsubstituted or substituted by 1 to 3 substituents selected from a group consisting of benzoyloxy; alkoxycarbonyl; alkyl; alkylsulfonyl; alkylcarbonylthio; alkoxycarbonylamino; and heterocycle which is unsubstituted or substituted by 1 to 3 substituents selected from a group consisting

of alkyl and oxo, and which nay include a double bond,

aryl unsubstituted or substituted by carboxy, or

aralkyl unsubstituted or substituted by alkylcarbonyloxy,

R⁶ is alkyl,

R⁴ is alkyl unsubstituted or substituted by 1 to 3 substituents selected from a group consisting of alkoxy unsubstituted or substituted by heteroaryl; carboxy; alkylthio; alkoxycarbonyl; alkyaminocarbonyl; biphenyl; aryl; aryloxy; aralkyloxy; alkylaryl; amino unsubstituted or substituted by a substituent(s) selected from a group consisting of alkyl and alkylaryl; cycloalkyl; cycloalkyloxy; alkylarylsulfonyl; heteroaryl unsubstituted or substituted by halogen; and heteroarylthio,

alkenyl unsubstituted or substituted by 1 to 3 substituents selected from a group consisting of halogen; aryl; and heteroaryl,

alkinyl unsubstituted or substituted by 1 to 3 substituents selected from a group consisting of alkoxy; alkenyloxy; alkinyloxy; aralkyloxy; aryl; and

heteroaryl,

—(CH₂)_{N}-cycloalkyl,

—(CH₂)_{N}-substituted cycloalkyl (this cycloalkyl is substituted by aryl which is unsubstituted or substituted by alkoxy or halogen; or fused with heteroaryl unsubstituted or substituted by halogen),

—(CH₂)_{N}-cycloalkenyl,

—(CH₂)_{N}-substituted cycloalkenyl (this cycloalkenyl is substituted by 1 to 3 substituents selected from a group consisting of alkyl and alkenyl),

—(CH₂)_{N}-aryl,

—(CH₂)_{N}-substituted aryl (this aryl is substituted by 1 to 5 substituents selected from a group consisting of nitro; cyano; hydroxy; halogen; alkyl; halogenoalkyl; alkoxy; halogenoalkoxy; alkylthio; halogenoalkylthio; alkylsulfonyl; alkoxycarbonyl; alkoxy carbonyloxy; amino unsubstituted or substituted by a substituent(s) selected from a group consisting of alkyl, alkoxyalkyl, alkenyl, cycloalkyl, and cycloalkylalkyl; aryloxy; aralkyloxy unsubstituted or substituted by alkoxy; and heterocycle),

—(CH₂)_{N}-heterocycle,

—(CH₂)_{N}-substituted heterocycle (this heterocycle is substituted by 1 to 3 substituents selected from a group consisting of oxo; nitro; alkyl; aralkyl;

and aryl unsubstituted or substituted by nitro),

—(CH₂)_{N}-heteroaryl, or

—(CH₂)_{N}-substituted heteroaryl (this heteroaryl is substituted by 1 to 3 substituents selected from a group consisting of oxo; nitro; halogen; alkyl; alkoxy; alkylthio; alkylarylsulfonyl; aryl; and heteroaryl unsubstituted or substituted by 1 to 3 substituents selected from a group consisting of alkyl and halogenoalkyl),

wherein N is an integer of 0 to 10, and when N is not 0, the —CH₂— group is unsubstituted or substituted by halogen, or its pharmaceutically acceptable salt, hydrate, solvate, or isomer including tautomer.

In the above definitions according to the present invention, the tern ‘halogen’ means a radical of fluorine, chlorine, bromine, or iodine, and the preferable halogen radical is fluorine, chlorine, or bromine.

The term ‘alkyl’ used alone or as a composite term with other term means straight-chain or branched, saturated aliphatic hydrocarbon radical having preferably 1 to 10 carbon atoms, more preferably 1 to 5 carbon atoms. The alkyl radical may include methyl, ethyl, n-propyl, isopropyl, n-butyl, isobutyl, sec-butyl, t-butyl, pentyl, isoamyl, n-hexyl, etc., but not restricted thereto.

The term ‘alkenyl’ used alone or as a composite term with other term means straight-chain or branched, mono- or poly-unsaturated, aliphatic hydrocarbon radical having preferably 2 to 10 carbon atoms, more preferably 2 to 5 carbon atoms. The alkenyl radical may include ethenyl, E-propenyl, Z-propenyl, E-isobutenyl, Z-isobutenyl, E-pentenyl, Z-pentenyl, E-hexenyl, Z-hexenyl, E,E-hexadienyl, E,Z-hexadienyl, Z,E-hexadienyl, Z,Z-hexadienyl, etc., but not restricted thereto.

The term ‘alkinyl’ used alone or as a composite term with other term means straight-chain or branched, mono- or poly-unsaturated, aliphatic hydrocarbon radical having preferably 2 to 10 carbon atoms, more preferably 2 to 5 carbon atoms, but necessarily having one or more triple bonds. The alkinyl radical may include ethinyl, propinyl, isobutinyl, pentinyl, hexinyl, hexeninyl, etc., but not restricted thereto.

The term ‘aryl’ used alone or as a composite term with other term means carbocyclic aromatic radical having preferably 6 to 14 carbon atoms, more preferably 6 to 10 carbon atoms, which my optionally be fused with further 1 to 3 aryls (for example, benzo-fused). The aryl radical may include phenyl, naphthyl, anthracenyl, etc., but not restricted thereto.

The term ‘cycloalkyl’ used alone or as a composite term with other term means nonaromatic saturated hydrocarbocyclic radical having preferably 3 to 8 carbon atoms, more preferably 5 to 6 carbon atoms, which my optionally be fused with further 1 to 3 cycloalkyls or aryls (for example, benzo-fused). The cycloalkyl radical may include cyclopropyl, cyclobutyl, cyclopentyl, cyclohexyl, cycloheptyl, indane, tetrahydronaphthalene, etc., but not restricted thereto.

The term ‘cycloalkenyl’ used alone or as a composite term with other term means nonaromatic unsaturated hydrocarbocyclic radical having preferably 5 to 7 carbon atoms, but necessarily having one or more carbon-carbon double bonds in the ring. The cycloalkenyl may be combined with any intracyclic carbon atom that forms a stable structure. The cycloalkenyl radical may include cyclopentenyl, cyclohexenyl, cyclopenadienyl, indenyl, etc., but not restricted thereto.

The term ‘heterocycle’ used alone or as a composite term with other term means 5 to 15-membered, preferably 5- to 6-membered, mono to tricyclic heterocycle, which is saturated or partially unsaturated, nonaromatic ring, and which may optionally be fused with further 1 to 3 cycloalkyls, aryls, heterocycles, or heteroaryls (for example, benzo-fused). The heterocycle radical includes 1 to 4 heteroatoms selected from a group consisting of nitrogen, oxygen, and sulfur, and the ‘nitrogen and sulfur atoms’ used in the present invention may be in any oxidized form or quaternary amine form. The heterocycle may be combined with any intracyclic carbon or heteroatom that forms a stable structure. The heterocycle radical may include imidazolidinyl, indazolinolyl, perhydropyridazyl, pyrrolinyl, pyrrolidinyl, piperidinyl, pyrazolinyl, piperazinyl, morpholinyl, thiamorpholinyl, β-carborinyl, thiazolidinyl, thiamorpholinyl sulfon, oxopiperidinyl, oxopyrrolidinyl, oxoazepinyl, azepinyl, furazanyl, tetrahydropyranyl, tetrahydrofuranyl, oxathiolyl, dithiolyl, tetrahydrothiophenyl, dioxanyl, dioxolanyl, tetrahydrofurotetrahydrofuranyl, tetrahydropyranotetrahydrofuranyl, tetrahydrofuro-dihydrofuranyl, tetrahydropyranodihydrofuranyl, dihydropyranyl, dihydrofuranyl, dihydrofurotetrahydrofuranyl, dihydropyranotetrahydrofuranyl, sulforanyl, etc., but not restricted thereto.

The term ‘heteroaryl’ used alone or as a composite term with other term means 3 to 7-membered, preferably 5- to 6-membered, mono to tricyclic heterocycle, which is an aromatic ring, and which may optionally be fused with further 1 to 3 cycloalkyls, aryls, heterocycles, or heteroaryls (for example, benzo-fused). The heteroaryl radical includes 1 to 4 heteroatoms selected from a group consisting of nitrogen, oxygen, and sulfur, and the ‘nitrogen and sulfur atoms’ used in the present invention may be in any oxidized form or quaternary amine form. The heteroaryl nay be combined with any intracyclic carbon or heteroatom that forms a stable structure. The heteroaryl radical may include benzimidizolyl, imidazolyl, quinolyl, isoquinolyl, indolyl, indazolyl, pyridazyl, pyridyl, pyrrolyl, pyrazolyl, pyrazinyl, quinoxolyl, pyranyl, pyrimidinyl, pyridazinyl, furyl, thienyl, triazolyl, thiazolyl, benzothiazolyl, thieno-thiazolyl, thieno-pyrazolyl, tetrazolyl, benzofuranyl, oxazolyl, benzoxazolyl, isoxazolyl, isothiazolyl, thiadiazolyl, thiophenyl, etc., but not restricted thereto.

### BEST MODE FOR CARRYING OUT THE INVENTION

Among the compounds of formula (1) showing a potent effect in the treatment or prevention of hepatitis C, preferred compounds are those wherein

X is O or S

R¹ is hydrogen or

alkyl unsubstituted or substituted by 1 to 3 substituents selected from a group consisting of halogen; alkoxy; alkoxycarbonyl; carboxy; and aryl,

R² and R³ each are

hydrogen or

alkyl substituted by 1 to 3 substituents selected from a group consisting of halogen; hydroxy; alkoxy; and aralkyloxy,

R⁴ is alkyl unsubstituted or substituted by 1 to 3 substituents selected from a group consisting of alkoxy; alkoxycarbonyl; biphenyl; aryl; aryloxy;

aralkyloxy; alkylaryl; amino unsubstituted or substituted by a substituent(s) selected from a group consisting of alkyl and alkylaryl; cycloalkyl; cycloalkyloxy; alkylarylsulfonyl; heteroaryl; and heteroarylthio,

alkenyl unsubstituted or substituted by 1 to 3 substituents selected from a group consisting of halogen; aryl; and heteroaryl,

alkinyl unsubstituted or substituted by 1 to 3 substituents selected from a group consisting of alkoxy; alkenyloxy; alkinyloxy; aralkyloxy; aryl; and

heteroaryl,

—(CH₂)_{N}-cycloalkyl,

—(CH₂)_{N}-substituted cycloalkenyl (this cycloalkenyl is substituted by 1 to 3 substituents selected from a group consisting of alkyl and alkenyl),

—(CH₂)_{N}-aryl,

—(CH₂)_{N}-substituted aryl (this aryl is substituted by 1 to 3 substituents selected from a group consisting of nitro; cyano; hydroxy; halogen; alkyl; halogenoalkyl; alkoxy; halogenoalkoxy; alkylthio; halogenoalkylthio; alkylsulfonyl; alkoxycarbonyl; alkoxycarbonyloxy; amino unsubstituted or substituted by a substituent(s) selected from a group consisting of alkyl and alkoxyalkyl; and aralkyloxy unsubstituted or substituted by alkoxy),

—(CH₂)_{N}-heterocycle,

—(CH₂)_{N}-substituted heterocycle (this heterocycle is substituted by 1 to 3 substituents selected from a group consisting of oxo; nitro; alkyl; aralkyl; and aryl unsubstituted or substituted by nitro),

—(CH₂)_{N}-heteroaryl, or

—(CH₂)_{N}-substituted heteroaryl (this heteroaryl is substituted by 1 to 3 substituents selected from a group consisting of oxo; halogen; alkyl; alkylthio; alkylarylsulfonyl; aryl; and heteroaryl unsubstituted or substituted by 1 to 3 substituents selected from a group consisting of alkyl and halogenoalkyl),

wherein N is an integer of 0 to 10.

More preferred compounds are those wherein

R¹ is hydrogen or unsubstituted alkyl, or

R² is hydrogen, or

R³ is hydrogen; or

alkyl substituted by halogen or hydroxy.

Particularly preferred compounds are those wherein

R³ is hydrogen, hydroxymethyl, or fluoromethyl.

The most preferred compounds are those wherein

X is O or S

R¹ is unsubstituted alkyl,

R² is hydrogen, and

R³ is hydrogen, hydroxymethyl, or fluoromethyl.

Typical examples of the compound of formula (1) of the present invention are the following:

6-methyl-2-thioxo-1,2,3,4-tetrahydro-pyrimidine-5-carboxylic acid benzyl ester (6);

6-methyl-2-thioxo-1,2,3,4-tetrahydro-pyrimidine-5-carboxylic acid 4-nitro-benzyl ester (7);

6-methyl-2-thioxo-1,2,3,4-tetrahydro-pyrimidine-5-carboxylic acid 4-methoxy-benzyl ester (8);

6-methyl-2-thioxo-1,2,3,4-tetrahydro-pyrimidine-5-carboxylic acid benzo[1,3]dioxol-5-ylmethyl ester (9);

6-methyl-2-thioxo-1,2,3,4-tetrahydro-pyrimidine-5-carboxylic acid naphthalen-1-ylmethyl ester (10);

6-methyl-2-thioxo-1,2,3,4-tetrahydro-pyrimidine-5-carboxylic acid 4-methoxycarbonyl-benzyl ester (11);

6-methyl-2-thioxo-1,2,3,4-tetrahydro-pyrimidine-5-carboxylic acid furan-2-ylmethyl ester (12);

6-methyl-2-thioxo-1,2,3,4-tetrahydro-pyrimidine-5-carboxylic acid 3-pyridin-4-yl-propyl ester (13);

6-methyl-2-thioxo-1,2,3,4-tetrahydro-pyrimidine-5-carboxylic acid 3-phenyl-propyl ester (14);

6-methyl-2-thioxo-1,2,3,4-tetrahydro-pyrimidine-5-carboxylic acid 2-benzyloxy-ethyl ester (15);

6-methyl-2-thioxo-1,2,3,4-tetrahydro-pyrimidine-5-carboxylic acid allyl ester (16);

6-naphthalen-1-yl-2-thioxo-1,2,3,4-tetrahydro-pyrimidine-5-carboxylic acid benzyl ester (17);

6-benzyl-2-thioxo-1,2,3,4-tetrahydro-pyrimidine-5-carboxylic acid benzyl ester (18);

6-phenethyl-2-thioxo-1,2,3,4-tetrahydro-pyrimidine-5-carboxylic acid benzyl ester (19);

6-methyl-2-thioxo-1,2,3,4-tetrahydro-pyrimidine-5-carboxylic acid biphenyl-4-ylmethyl ester (22);

6-methyl-2-thioxo-1,2,3,4-tetrahydro-pyrimidine-5-carboxylic acid (R)-1-phenyl-ethyl ester (23);

6-methyl-2-thioxo-1,2,3,4-tetrahydro-pyrimidine-5-carboxylic acid 2-(4-methyl-thiazol-5-yl)-ethyl ester (24);

6-propyl-2-thioxo-1,2,3,4-tetrahydro-pyrimidine-5-carboxylic acid benzyl ester (25);

6-methyl-2-thioxo-1,2,3,4-tetrahydro-pyrimidine-5-carboxylic acid (R,S)-1-phenyl-ethyl ester (26);

6-methyl-2-thioxo-1,2,3,4-tetrahydro-pyrimidine-5-carboxylic acid 1-methyl-1-phenyl-ethyl ester (27);

6-methyl-2-thioxo-1,2,3,4-tetrahydro-pyrimidine-5-carboxylic acid cyclohexylmethyl ester (29);

6-methyl-2-thioxo-1,2,3,4-tetrahydro-pyrimidine-5-carboxylic acid hexyl ester (30);

6-methyl-2-thioxo-1,2,3,4-tetrahydro-pyrimidine-5-carboxylic acid 8-phenyl-octyl ester (33);

6-methyl-2-thioxo-1,2,3,4-tetrahydro-pyrimidine-5-carboxylic acid phenethyl ester (34);

6-methyl-2-thioxo-1,2,3,4-tetrahydro-pyrimidine-5-carboxylic acid 1-methyl-heptyl ester (35);

6-methyl-2-thioxo-1,2,3,4-tetrahydro-pyrimidine-5-carboxylic acid 3-(3,4-dimethoxy-phenyl)-propyl ester (36);

6-methyl-2-thioxo-1,2,3,4-tetrahydro-pyrimidine-5-carboxylic acid cyclohexyl ester (37);

6-methyl-2-thioxo-1,2,3,4-tetrahydro-pyrimidine-5-carboxylic acid 1-methyl-pentyl ester (39);

6-methyl-2-thioxo-1,2,3,4-tetrahydro-pyrimidine-5-carboxylic acid 3,4-dimethoxy-benzyl ester (40);

6-methyl-2-thioxo-1,2,3,4-tetrahydro-pyrimidine-5-carboxylic acid 2,4-dichloro-benzyl ester (41);

6-methyl-2-thioxo-1,2,3,4-tetrahydro-pyrimidine-5-carboxylic acid 1-methyl-3-phenyl-propyl ester (42);

6-methyl-2-thioxo-1,2,3,4-tetrahydro-pyrimidine-5-carboxylic acid 4-chloro-benzyl ester (43);

2-thioxo-1,2,3,4-tetrahydro-pyrimidine-5-carboxylic acid benzyl ester (44);

6-methyl-2-thioxo-1,2,3,4-tetrahydro-pyrimidine-5-carboxylic acid 4-t-butyl-benzyl ester (45);

6-methyl-2-thioxo-1,2,3,4-tetrahydro-pyrimidine-5-carboxylic acid cyclopropylmethyl ester (46);

6-methyl-2-thioxo-1,2,3,4-tetrahydro-pyrimidine-5-carboxylic acid 1-ethyl-hexyl ester (47);

6-methyl-2-thioxo-1,2,3,4-tetrahydro-pyrimidine-5-carboxylic acid 3-cyano-benzyl ester (48);

6-methyl-2-thioxo-1,2,3,4-tetrahydro-pyrimidine-5-carboxylic acid 4-benzyloxy-benzyl ester (49);

6-methyl-2-thioxo-1,2,3,4-tetrahydro-pyrimidine-5-carboxylic acid 3-benzyloxy-benzyl ester (50);

6-methyl-2-thioxo-1,2,3,4-tetrahydro-pyrimidine-5-carboxylic acid 4-butyl-benzyl ester (51);

6-methyl-2-thioxo-1,2,3,4-tetrahydro-pyrimidine-5-carboxylic acid 4-methylsulfinyl-benzyl ester (52);

6-methyl-2-thioxo-1,2,3,4-tetrahydro-pyrimidine-5-carboxylic acid 3-methyl-but-2-enyl ester (53);

6-methyl-2-thioxo-1,2,3,4-tetrahydro-pyrimidine-5-carboxylic acid 3-phenyl-allyl ester (54);

6-methyl-2-thioxo-1,2,3,4-tetrahydro-pyrimidine-5-carboxylic acid 1-vinyl-but-3-enyl ester (55);

6-methyl-2-thioxo-1,2,3,4-tetrahydro-pyrimidine-5-carboxylic acid 3,7-dimethyl-octa-2,6-dienyl ester (56);

6-methyl-2-thioxo-1,2,3,4-tetrahydro-pyrimidine-5-carboxylic acid phenyl-o-tolyl-methyl ester (57);

6-methyl-2-thioxo-1,2,3,4-tetrahydro-pyrimidine-5-carboxylic acid 4-benzyloxy-3-methoxy-benzyl ester (58);

6-methyl-2-thioxo-1,2,3,4-tetrahydro-pyrimidine-5-carboxylic acid 2-(ethyl-m-tolyl-amino)-ethyl ester (59);

6-methyl-2-thioxo-1,2,3,4-tetrahydro-pyrimidine-5-carboxylic acid 4-bromo-benzyl ester (60);

6-methyl-2-thioxo-1,2,3,4-tetrahydro-pyrimidine-5-carboxylic acid 4-fluoro-benzyl ester (61);

6-methoxy-2-thioxo-1,2,3,4-tetrahydro-pyrimidine-5-carboxylic acid benzyl ester (63);

6-methoxymethyl-2-thioxo-1,2,3,4-tetrahydro-pyrimidine-5-carboxylic acid benzyl ester (64);

6-methyl-2-thioxo-1,2,3,4-tetrahydro-pyrimidine-5-carboxylic acid 2-methoxy-benzyl ester (65);

6-methyl-2-thioxo-1,2,3,4-tetrahydro-pyrimidine-5-carboxylic acid 2-ethoxy-benzyl ester (66);

6-methyl-2-thioxo-1,2,3,4-tetrahydro-pyrimidine-5-carboxylic acid cyclopentyl ester (67);

6-methyl-2-thioxo-1,2,3,4-tetrahydro-pyrimidine-5-carboxylic acid cyclopentylmethyl ester (68);

6-methyl-2-thioxo-1,2,3,4-tetrahydro-pyrimidine-5-carboxylic acid 2,4-difluoro-benzyl ester (69);

6-methyl-2-thioxo-1,2,3,4-tetrahydro-pyrimidine-5-carboxylic acid 2-chloro-4-fluoro-benzyl ester (70);

6-methyl-2-thioxo-1,2,3,4-tetrahydro-pyrimidine-5-carboxylic acid 4-ethoxy-benzyl ester (71);

6-methyl-2-thioxo-1,2,3,4-tetrahydro-pyrimidine-5-carboxylic acid 4-isobutoxy-benzyl ester (72);

6-methyl-2-thioxo-1,2,3,4-tetrahydro-pyrimidine-5-carboxylic acid 4-methanesulfonyl-benzyl ester (74);

6-methyl-2-thioxo-1,2,3,4-tetrahydro-pyrimidine-5-carboxylic acid 3-iodo-benzyl ester (75);

6-methyl-2-thioxo-1,2,3,4-tetrahydro-pyrimidine-5-carboxylic acid 3,4-dimethyl-benzyl ester (76);

6-methyl-2-thioxo-1,2,3,4-tetrahydro-pyrimidine-5-carboxylic acid 3-nitro-benzyl ester (77);

6-methyl-2-thioxo-1,2,3,4-tetrahydro-pyrimidine-5-carboxylic acid 4-propoxy-benzyl ester (78);

6-methyl-2-thioxo-1,2,3,4-tetrahydro-pyrimidine-5-carboxylic acid 4-dimethylamino-benzyl ester (79);

6-methyl-2-thioxo-1,2,3,4-tetrahydro-pyrimidine-5-carboxylic acid cyclobutylmethyl ester (80);

6-methyl-2-thioxo-1,2,3,4-tetrahydro-pyrimidine-5-carboxylic acid 3,5-dimethoxy-benzyl ester (81);

6-methyl-2-thioxo-1,2,3,4-tetrahydro-pyrimidine-5-carboxylic acid 3,5-dimethyl-benzyl ester (82);

6-methyl-2-thioxo-1,2,3,4-tetrahydro-pyrimidine-5-carboxylic acid 3-fluoro-benzyl ester (83);

6-methyl-2-thioxo-1,2,3,4-tetrahydro-pyrimidine-5-carboxylic acid 2-chloro-benzyl ester (84);

6-methyl-2-thioxo-1,2,3,4-tetrahydro-pyrimidine-5-carbothioic acid S-benzyl ester (85);

6-methyl-2-thioxo-1,2,3,4-tetrahydro-pyrimidine-5-carboxylic acid 1-benzyl-piperidin-4-yl ester (86);

2-thioxo-1,2,3,6-tetrahydro-pyrimidine-4,5-dicarboxylic acid dibenzyl ester (87);

6-methyl-2-thioxo-1,2,3,4-tetrahydro-pyrimidine-5-carboxylic acid 3-(4-methoxy-phenyl)-propyl ester (88);

6-methyl-2-thioxo-1,2,3,4-tetrahydro-pyrimidine-5-carboxylic acid 4-methyl-benzyl ester (89);

6-methyl-2-thioxo-1,2,3,4-tetrahydro-pyrimidine-5-carboxylic acid 3-methyl-benzyl ester (90);

6-methyl-2-thioxo-1,2,3,4-tetrahydro-pyrimidine-5-carboxylic acid 3-chloro-benzyl ester (91);

6-methyl-2-thioxo-1,2,3,4-tetrahydro-pyrimidine-5-carboxylic acid 2-(4-methoxy-phenyl)-ethyl ester (92);

6-methyl-2-thioxo-1,2,3,4-tetrahydro-pyrimidine-5-carboxylic acid 2-(4-fluoro-phenyl)-ethyl ester (93);

6-methyl-2-thioxo-1,2,3,4-tetrahydro-pyrimidine-5-carboxylic acid 2-(4-chloro-phenyl)-ethyl ester (94);

6-methyl-2-thioxo-1,2,3,4-tetrahydro-pyrimidine-5-carboxylic acid 2-(4-dimethylamino-phenyl)-ethyl ester (95);

6-methyl-2-thioxo-1,2,3,4-tetrahydro-pyrimidine-5-carboxylic acid (s)-(−)-4-isopropenyl-cyclohex-1-enylmethyl ester (96);

6-methyl-2-thioxo-1,2,3,4-tetrahydro-pyrimidine-5-carboxylic acid 6,6-dimethyl-bicyclo[3.1.1]hept-2-en-2-ylmethyl ester (97);

6-methyl-2-thioxo-1,2,3,4-tetrahydro-pyrimidine-5-carboxylic acid 2-chloro-allyl ester (98);

6-methyl-2-thioxo-1,2,3,4-tetrahydro-pyrimidine-5-carboxylic acid 3-bromo-but-3-enyl ester (99);

6-methyl-2-thioxo-1,2,3,4-tetrahydro-pyrimidine-5-carboxylic acid 2-trifluoromethyl-benzyl ester (100);

6-methyl-2-thioxo-1,2,3,4-tetrahydro-pyrimidine-5-carboxylic acid 3-trifluoromethyl-benzyl ester (101);

6-methyl-2-thioxo-1,2,3,4-tetrahydro-pyrimidine-5-carboxylic acid 2-(1H-indol-3-yl)-ethyl ester (102);

6-methyl-2-thioxo-1,2,3,4-tetrahydro-pyrimidine-5-carboxylic acid 3-(4-fluoro-phenyl)-propyl ester (103);

6-methyl-2-thioxo-1,2,3,4-tetrahydro-pyrimidine-5-carboxylic acid 3-(2-methoxy-phenyl)-propyl ester (104);

6-methyl-2-thioxo-1,2,3,4-tetrahydro-pyrimidine-5-carboxylic acid 3-(4-methylsulfanyl-phenyl)-propyl ester (105);

6-methyl-2-thioxo-1,2,3,4-tetrahydro-pyrimidine-5-carboxylic acid 3-(3-trifluoromethoxy-phenyl)-propyl ester (106);

6-methyl-2-thioxo-1,2,3,4-tetrahydro-pyrimidine-5-carboxylic acid 3-(4-trifluoromethyl-phenyl)-propyl ester (107);

6-methyl-2-thioxo-1,2,3,4-tetrahydro-pyrimidine-5-carboxylic acid 3-(4-benzyloxy-phenyl)-propyl ester (108);

4-methoxymethyl-6-methyl-2-thioxo-1,2,3,4-tetrahydro-pyrimidine-5-carboxylic acid benzyl ester (109);

6-methyl-2-thioxo-1,2,3,4-tetrahydro-pyrimidine-5-carboxylic acid 2-methylsulfanyl-benzyl ester (110);

6-methyl-2-thioxo-1,2,3,4-tetrahydro-pyrimidine-5-carboxylic acid 2-isobutylsulfanyl-benzyl ester (111);

6-methyl-2-thioxo-1,2,3,4-tetrahydro-pyrimidine-5-carboxylic acid 2-methanesulfonyl-benzyl ester (112);

6-methyl-2-thioxo-1,2,3,4-tetrahydro-pyrimidine-5-carboxylic acid 2-(2-methyl-propane-1-sulfonyl)-benzyl ester (113);

6-methyl-2-thioxo-1,2,3,4-tetrahydro-pyrimidine-5-carboxylic acid 4-trifluoromethyl-benzyl ester (114);

6-methyl-2-thioxo-1,2,3,4-tetrahydro-pyrimidine-5-carboxylic acid 2-(2-methoxy-phenyl)-ethyl ester (115);

6-methyl-2-thioxo-1,2,3,4-tetrahydro-pyrimidine-5-carboxylic acid 2-(2-fluoro-phenyl)-ethyl ester (116);

6-methyl-2-thioxo-1,2,3,4-tetrahydro-pyrimidine-5-carboxylic acid 2-(3-chloro-phenyl)-ethyl ester (117);

6-methyl-2-thioxo-1,2,3,4-tetrahydro-pyrimidine-5-carboxylic acid 2-(2-chloro-phenyl)-ethyl ester (118);

6-methyl-2-thioxo-1,2,3,4-tetrahydro-pyrimidine-5-carboxylic acid 2-(4-nitro-phenyl)-ethyl ester (119);

6-methyl-2-thioxo-1,2,3,4-tetrahydro-pyrimidine-5-carboxylic acid 3-methoxy-benzyl ester (120);

6-methyl-2-thioxo-1,2,3,4-tetrahydro-pyrimidine-5-carbothioic acid S-(4-methoxy-benzyl)ester (121);

6-methyl-2-thioxo-1,2,3,4-tetrahydro-pyrimidine-5-carboxylic acid 2,4-dimethyl-benzyl ester (122);

6-methyl-2-thioxo-1,2,3,4-tetrahydro-pyrimidine-5-carboxylic acid indan-1-yl ester (123);

6-methyl-2-thioxo-1,2,3,4-tetrahydro-pyrimidine-5-carboxylic acid indan-2-yl ester (124);

6-methyl-2-thioxo-1,2,3,4-tetrahydro-pyrimidine-5-carboxylic acid 2-(indan-1-yloxy)-ethyl ester (125);

6-methyl-2-thioxo-1,2,3,4-tetrahydro-pyrimidine-5-carboxylic acid 2-indan-1-yl-ethyl ester (126);

6-methyl-2-thioxo-1,2,3,4-tetrahydro-pyrimidine-5-carboxylic acid 1,1-dioxo-1H-11⁶-benzo[b]thiophen-2-ylmethyl ester (127);

6-methyl-2-thioxo-1,2,3,4-tetrahydro-pyrimidine-5-carboxylic acid 5-(4-nitro-phenyl)-furan-2-ylmethyl ester (128);

6-methyl-2-thioxo-1,2,3,4-tetrahydro-pyrimidine-5-carboxylic acid but-3-inyl ester (129);

6-methyl-2-thioxo-1,2,3,4-tetrahydro-pyrimidine-5-carboxylic acid pent-4-inyl ester (130);

6-methyl-2-thioxo-1,2,3,4-tetrahydro-pyrimidine-5-carboxylic acid 3,5-dichloro-benzyl ester (131);

6-methyl-2-thioxo-1,2,3,4-tetrahydro-pyrimidine-5-carboxylic acid 5-chloro-2-methoxy-benzyl ester (132);

6-methyl-2-thioxo-1,2,3,4-tetrahydro-pyrimidine-5-carboxylic acid 2-o-tolyl-ethyl ester (133);

6-methyl-2-thioxo-1,2,3,4-tetrahydro-pyrimidine-5-carboxylic acid 3,5-difluoro-benzyl ester (134);

6-methyl-2-thioxo-1,2,3,4-tetrahydro-pyrimidine-5-carboxylic acid 3-methyl-4-nitro-benzyl ester (135);

6-methyl-2-thioxo-1,2,3,4-tetrahydro-pyrimidine-5-carboxylic acid 3-nitro-4-methyl-benzyl ester (136);

6-methyl-2-thioxo-1,2,3,4-tetrahydro-pyrimidine-5-carboxylic acid 2,4-dimethoxy-3-methyl-benzyl ester (137);

6-methyl-2-thioxo-1,2,3,4-tetrahydro-pyrimidine-5-carboxylic acid 4-methoxy-2,3-dimethyl-benzyl ester (138);

6-methyl-2-thioxo-1,2,3,4-tetrahydro-pyrimidine-5-carboxylic acid thiophen-3-ylmethyl ester (139);

6-methyl-2-thioxo-1,2,3,4-tetrahydro-pyrimidine-5-carboxylic acid 6-nitro-benzo[1,3]dioxol-5-ylmethyl ester (140);

6-methyl-2-thioxo-1,2,3,4-tetrahydro-pyrimidine-5-carboxylic acid 3-bromo-4-methoxy-benzyl ester (141);

6-methyl-2-thioxo-1,2,3,4-tetrahydro-pyrimidine-5-carboxylic acid 2,4,6-trimethyl-benzyl ester (142);

6-methyl-2-thioxo-1,2,3,4-tetrahydro-pyrimidine-5-carboxylic acid 5-chloro-2-nitro-benzyl ester (143);

2-thioxo-6-trifluoromethyl-1,2,3,4-tetrahydro-pyrimidine-5-carboxylic acid benzyl ester (146);

6-methyl-2-thioxo-1,2,3,4-tetrahydro-pyrimidine-5-carboxylic acid 9-ethyl-9H-carbazol-3-ylmethyl ester (147);

6-methyl-2-thioxo-1,2,3,4-tetrahydro-pyrimidine-5-carboxylic acid 3,5-bis-trifluoromethyl-benzyl ester (148);

6-methyl-2-thioxo-1,2,3,4-tetrahydro-pyrimidine-5-carboxylic acid 2,5-dimethyl-benzyl ester (149);

6-methyl-2-thioxo-1,2,3,4-tetrahydro-pyrimidine-5-carboxylic acid 3,5-diethoxy-benzyl ester (150);

6-methyl-2-thioxo-1,2,3,4-tetrahydro-pyrimidine-5-carboxylic acid bicyclo[2.2.1]hept-2-ylmethyl ester (151);

6-methyl-2-thioxo-1,2,3,4-tetrahydro-pyrimidine-5-carboxylic acid 4-ethyl-benzyl ester (152);

6-methyl-2-thioxo-1,2,3,4-tetrahydro-pyrimidine-5-carboxylic acid thiophen-2-ylmethyl ester (153);

6-methyl-2-thioxo-1,2,3,4-tetrahydro-pyrimidine-5-carboxylic acid pyridin-2-ylmethyl ester (154);

6-methyl-2-thioxo-1,2,3,4-tetrahydro-pyrimidine-5-carboxylic acid pyridin-4-ylmethyl ester (155);

6-methyl-2-thioxo-1,2,3,4-tetrahydro-pyrimidine-5-carboxylic acid 2,3-dimethyl-benzyl ester (156);

6-methyl-2-thioxo-1,2,3,4-tetrahydro-pyrimidine-5-carboxylic acid hex-3-inyl ester (157);

6-methyl-2-thioxo-1,2,3,4-tetrahydro-pyrimidine-5-carboxylic acid 3-pyridin-3-yl-propyl ester (158);

6-methyl-2-thioxo-1,2,3,4-tetrahydro-pyrimidine-5-carboxylic acid prop-2-inyl ester (159);

6-methyl-2-thioxo-1,2,3,4-tetrahydro-pyrimidine-5-carboxylic acid pent-3-inyl ester (160);

6-methyl-2-thioxo-1,2,3,4-tetrahydro-pyrimidine-5-carboxylic acid tetrahydrofuran-2-ylmethyl ester (161);

6-methyl-2-thioxo-1,2,3,4-tetrahydro-pyrimidine-5-carboxylic acid tetrahydrofuran-3-ylmethyl ester (162);

6-methyl-2-thioxo-1,2,3,4-tetrahydro-pyrimidine-5-carboxylic acid 2-phenoxy-ethyl ester (163);

6-methyl-2-thioxo-1,2,3,4-tetrahydro-pyrimidine-5-carboxylic acid 3-trifluoromethylsulfanyl-benzyl ester (164);

6-methyl-2-thioxo-1,2,3,4-tetrahydro-pyrimidine-5-carboxylic acid 2-(1-methyl-1H-indol-3-yl)-ethyl ester (165);

6-(3-ethoxycarbonyl-propyl)-2-thioxo-1,2,3,4-tetrahydro-pyrimidine-5-carboxylic acid benzyl ester (169);

6-(3-carboxy-propyl)-2-thioxo-1,2,3,4-tetrahydro-pyrimidine-5-carboxylic acid benzyl ester (170);

6-methyl-2-thioxo-1,2,3,4-tetrahydro-pyrimidine-5-carboxylic acid 2-m-tolyl-ethyl ester (172);

6-methyl-2-thioxo-1,2,3,4-tetrahydro-pyrimidine-5-carboxylic acid 2-p-tolyl-ethyl ester (173);

6-methyl-2-thioxo-1,2,3,4-tetrahydro-pyrimidine-5-carboxylic acid 2,5-dimethoxy-benzyl ester (174);

6-methyl-2-thioxo-1,2,3,4-tetrahydro-pyrimidine-5-carboxylic acid 2-(3,5-dimethylphenyl)-ethyl ester (175);

6-methyl-2-thioxo-1,2,3,4-tetrahydro-pyrimidine-5-carboxylic acid 4-methoxy-3-methyl-benzyl ester (176);

6-methyl-2-thioxo-1,2,3,4-tetrahydro-pyrimidine-5-carboxylic acid 2-fluoro-4-methoxy-benzyl ester (177);

6-methyl-2-thioxo-1,2,3,4-tetrahydro-pyrimidine-5-carboxylic acid 3-bromo-4,5-dimethoxy-benzyl ester (178);

6-methyl-2-thioxo-1,2,3,4-tetrahydro-pyrimidine-5-carboxylic acid 4-methoxy-3,5-dimethyl-benzyl ester (180);

6-methyl-2-thioxo-1,2,3,4-tetrahydro-pyrimidine-5-carboxylic acid 3,5-dimethyl-1-phenyl-1H-pyrazol-4-ylmethyl ester (181);

6-methyl-2-thioxo-1,2,3,4-tetrahydro-pyrimidine-5-carboxylic acid 2-nitro-benzyl ester (183);

6-methyl-2-thioxo-1,2,3,4-tetrahydro-pyrimidine-5-carboxylic acid 2,6-dimethyl-benzyl ester (184);

6-methyl-2-thioxo-1,2,3,4-tetrahydro-pyrimidine-5-carboxylic acid 5-bromo-2-methoxy-benzyl ester (185);

6-methyl-2-thioxo-1,2,3,4-tetrahydro-pyrimidine-5-carboxylic acid 3-phenyl-prop-2-inyl ester (192);

6-methyl-2-thioxo-1,2,3,4-tetrahydro-pyrimidine-5-carboxylic acid 2,3-dibromo-allyl ester (193);

6-methyl-2-thioxo-1,2,3,4-tetrahydro-pyrimidine-5-carboxylic acid 2,3-dihydro-benzo[1,4]dioxin-2-ylmethyl ester (195);

2-thioxo-1,2,3,6-tetrahydro-pyrimidine-4,5-dicarboxylic acid 5-benzyl ester 4-methyl ester (196);

6-methyl-2-thioxo-1,2,3,4-tetrahydro-pyrimidine-5-carboxylic acid 5-ethoxycarbonyl-pentyl ester (197);

6-methyl-2-thioxo-1,2,3,4-tetrahydro-pyrimidine-5-carboxylic acid 9-methoxycarbonyl-nonyl ester (198);

6-methyl-2-thioxo-1,2,3,4-tetrahydro-pyrimidine-5-carboxylic acid 4-trifluoromethylsulfanyl-benzyl ester (199);

6-methyl-2-thioxo-1,2,3,4-tetrahydro-pyrimidine-5-carboxylic acid dec-3-inyl ester (200);

6-methyl-2-thioxo-1,2,3,4-tetrahydro-pyrimidine-5-carboxylic acid 2-(toluene-4-sulfonyl)-ethyl ester (201);

6-methyl-2-thioxo-1,2,3,4-tetrahydro-pyrimidine-5-carboxylic acid 3-methyl-thiophen-2-ylmethyl ester (202);

6-methyl-2-thioxo-1,2,3,4-tetrahydro-pyrimidine-5-carboxylic acid 5-bromo-thiophen-2-ylmethyl ester (203);

6-methyl-2-thioxo-1,2,3,4-tetrahydro-pyrimidine-5-carboxylic acid 4-bromo-thiophen-2-ylmethyl ester (204);

6-methyl-2-thioxo-1,2,3,4-tetrahydro-pyrimidine-5-carboxylic acid 5-ethyl-thiophen-2-ylmethyl ester (205);

6-methyl-2-thioxo-1,2,3,4-tetrahydro-pyrimidine-5-carboxylic acid 2-thiophen-2-yl-ethyl ester (206);

6-methyl-2-thioxo-1,2,3,4-tetrahydro-pyrimidine-5-carboxylic acid 3-chloro-4-methyl-thiophen-2-ylmethyl ester (207);

6-methyl-2-thioxo-1,2,3,4-tetrahydro-pyrimidine-5-carboxylic acid 2-thiophen-3-yl-ethyl ester (208);

6-methyl-2-thioxo-1,2,3,4-tetrahydro-pyrimidine-5-carboxylic acid 2-{4-[(2-methoxy-ethyl)-methyl-amino]-phenyl}-ethyl ester (209);

6-methyl-2-thioxo-1,2,3,4-tetrahydro-pyrimidine-5-carboxylic acid 2-thiophen-2-yl-thiazol-5-ylmethyl ester (210);

4-fluoromethyl-6-methyl-2-thioxo-1,2,3,4-tetrahydro-pyrimidine-5-carboxylic acid benzyl ester (211);

6-methyl-2-thioxo-1,2,3,4-tetrahydro-pyrimidine-5-carboxylic acid 3-methyl-thieno[2,3-c]isothiazol-5-ylmethyl ester (213);

6-methyl-2-thioxo-1,2,3,4-tetrahydro-pyrimidine-5-carboxylic acid 4-benzyloxy-but-2-inyl ester (214);

6-methyl-2-thioxo-1,2,3,4-tetrahydro-pyrimidine-5-carboxylic acid 4-methoxy-but-2-inyl ester (215);

6-methyl-2-thioxo-1,2,3,4-tetrahydro-pyrimidine-5-carboxylic acid 2,6-dimethyl-thieno[3,2-d]thiazol-5-ylmethyl ester (216);

6-methyl-2-thioxo-1,2,3,4-tetrahydro-pyrimidine-5-carboxylic acid 5-methylsulfanyl-thiophen-2-ylmethyl ester (217);

6-methyl-2-thioxo-1,2,3,4-tetrahydro-pyrimidine-5-carboxylic acid[2,2′]bithiophenyl-5-ylmethyl ester (218);

6-methyl-2-thioxo-1,2,3,4-tetrahydro-pyrimidine-5-carboxylic acid benzo[b]thiophen-2-ylmethyl ester (219);

6-methyl-2-thioxo-1,2,3,4-tetrahydro-pyrimidine-5-carboxylic acid 3-pyrrol-1-yl-thiophen-2-ylmethyl ester (220);

6-methyl-2-thioxo-1,2,3,4-tetrahydro-pyrimidine-5-carboxylic acid 5-(2-methyl-thiazol-5-yl)-thiophen-2-ylmethyl ester (221);

6-methyl-2-thioxo-1,2,3,4-tetrahydro-pyrimidine-5-carboxylic acid 5-(1-methyl-5-trifluoromethyl-1H-pyrazol-3-yl)-thiophen-2-ylmethyl ester (222);

6-methyl-2-thioxo-1,2,3,4-tetrahydro-pyrimidine-5-carboxylic acid 1,3-dimethyl-1H-thieno[2,3-c]pyrazol-5-ylmethyl ester (223);

6-methyl-2-thioxo-1,2,3,4-tetrahydro-pyrimidine-5-carboxylic acid 2-(benzothiazol-2-ylsulfanyl)-ethyl ester (224);

6-methyl-2-thioxo-1,2,3,4-tetrahydro-pyrimidine-5-carboxylic acid benzotriazol-1-ylmethyl ester (225);

6-methyl-2-thioxo-1,2,3,4-tetrahydro-pyrimidine-5-carboxylic acid benzothiazol-2-ylmethyl ester (226);

6-methyl-2-thioxo-1,2,3,4-tetrahydro-pyrimidine-5-carboxylic acid 4-(4-methoxy-benzyloxy)-benzyl ester (227);

6-methyl-2-thioxo-1,2,3,4-tetrahydro-pyrimidine-5-carboxylic acid 4-t-butoxycarbonyloxy-benzyl ester (228);

6-methyl-2-thioxo-1,2,3,4-tetrahydro-pyrimidine-5-carboxylic acid 4-hydroxy-benzyl ester (229);

6-methyl-2-thioxo-1,2,3,4-tetrahydro-pyrimidine-5-carboxylic acid 1-(toluene-4-sulfonyl)-1H-pyrrol-3-ylmethyl ester (230);

6-methyl-2-thioxo-1,2,3,4-tetrahydro-pyrimidine-5-carboxylic acid 2-methyl-benzyl ester (231);

6-methyl-2-thioxo-1,2,3,4-tetrahydro-pyrimidine-5-carboxylic acid 5-methyl-thiophen-2-ylmethyl ester (232);

6-methyl-2-thioxo-1,2,3,4-tetrahydro-pyrimidine-5-carboxylic acid 5-chloro-thiophen-2-ylmethyl ester (233);

6-methyl-2-thioxo-4-trifluoromethyl-1,2,3,4-tetrahydro-pyrimidine-5-carboxylic acid benzyl ester (234);

6-methyl-2-thioxo-1,2,3,4-tetrahydro-pyrimidine-5-carboxylic acid thieno[2,3-b]thiophen-2-ylmethyl ester (235);

6-methyl-2-thioxo-1,2,3,4-tetrahydro-pyrimidine-5-carboxylic acid 4-ethoxy-but-2-inyl ester (236);

6-methyl-2-thioxo-1,2,3,4-tetrahydro-pyrimidine-5-carboxylic acid 4-propoxy-but-2-inyl ester (237);

6-methyl-2-thioxo-1,2,3,4-tetrahydro-pyrimidine-5-carboxylic acid benzoxazol-2-ylmethyl ester (238);

6-methyl-2-thioxo-1,2,3,4-tetrahydro-pyrimidine-5-carboxylic acid 3-thiophen-2-yl-prop-2-inyl ester (239);

6-methyl-2-thioxo-1,2,3,4-tetrahydro-pyrimidine-5-carboxylic acid 3-thiophen-3-yl-prop-2-inyl ester (240);

6-methyl-2-thioxo-1,2,3,4-tetrahydro-pyrimidine-5-carboxylic acid 1-methyl-1H-benzoimidazol-2-ylmethyl ester (241);

6-methyl-2-thioxo-1,2,3,4-tetrahydro-pyrimidine-5-carboxylic acid 3-methyl-3H-benzotriazol-5-ylmethyl ester (242);

6-methyl-2-thioxo-1,2,3,4-tetrahydro-pyrimidine-5-carboxylic acid 3-thiophen-3-yl-propyl ester (243);

6-methyl-2-thioxo-1,2,3,4-tetrahydro-pyrimidine-5-carboxylic acid 3-thiophen-3-yl-allyl ester (244);

6-methyl-2-thioxo-1,2,3,4-tetrahydro-pyrimidine-5-carboxylic acid 4-allyloxy-but-2-inyl ester (245);

6-methyl-2-thioxo-1,2,3,4-tetrahydro-pyrimidine-5-carboxylic acid 4-(1-methyl-prop-2-inyloxy)-but-2-inyl ester (246);

6-methyl-2-thioxo-1,2,3,4-tetrahydro-pyrimidine-5-carboxylic acid 2-(2-bromo-thiophen-3-yl)-ethyl ester (247);

6-methyl-2-thioxo-1,2,3,4-tetrahydro-pyrimidine-5-carboxylic acid benzothiazol-6-ylmethyl ester (248);

6-methyl-2-thioxo-1,2,3,4-tetrahydro-pyrimidine-5-carboxylic acid 2-(2-fluoro-thiophen-3-yl)-ethyl ester (249);

6-methyl-2-thioxo-1,2,3,4-tetrahydro-pyrimidine-5-carboxylic acid 3-furan-2-yl-propyl ester (250);

6-methyl-2-thioxo-1,2,3,4-tetrahydro-pyrimidine-5-carboxylic acid 3-furan-2-allyl ester (251);

4-fluoromethyl-6-methyl-2-thioxo-1,2,3,4-tetrahydro-pyrimidine-5-carboxylic acid 2-thiophen-3-yl-ethyl ester (252);

4-fluoromethyl-6-methyl-2-thioxo-1,2,3,4-tetrahydro-pyrimidine-5-carboxylic acid thiophen-3-ylmethyl ester (253);

6-methyl-2-thioxo-1,2,3,4-tetrahydro-pyrimidine-5-carboxylic acid 2-furan-3-yl-ethyl ester (254);

6-methyl-2-thioxo-1,2,3,4-tetrahydro-pyrimidine-5-carboxylic acid 2-thiophen-3-yl-1-methyl-ethyl ester (255);

6-methyl-2-thioxo-1,2,3,4-tetrahydro-pyrimidine-5-carboxylic acid 2-methyl-benzoxazol-6-ylmethyl ester (256);

6-methyl-2-thioxo-1,2,3,4-tetrahydro-pyrimidine-5-carboxylic acid 2-(3-methyl-thiophen-2-yl)-ethyl ester (257);

6-methyl-2-thioxo-1,2,3,4-tetrahydro-pyrimidine-5-carboxylic acid 2-(5-methyl-thiophen-2-yl)-ethyl ester (258);

6-methyl-2-thioxo-1,2,3,4-tetrahydro-pyrimidine-5-carboxylic acid 2-(4-fluoro-phenyl)-ethyl ester (259);

2-thioxo-1,2,3,4-tetrahydro-pyrimidine-5-carboxylic acid 2-thiophen-3-yl-ethyl ester (261);

4-fluoromethyl-6-methyl-2-thioxo-1,2,3,4-tetrahydro-pyrimidine-5-carboxylic acid 2-ethoxy-benzyl ester (263);

6-methyl-2-thioxo-1,2,3,4-tetrahydro-pyrimidine-5-carboxylic acid 5-ethyl-furan-2-ylmethyl ester (264);

6-methyl-2-thioxo-1,2,3,4-tetrahydro-pyrimidine-5-carboxylic acid 2-benzoxazol-2-yl-ethyl ester (265);

6-methyl-2-thioxo-1,2,3,4-tetrahydro-pyrimidine-5-carboxylic acid 2-(5-ethyl-thiophen-2-yl)-ethyl ester (266);

6-methyl-2-thioxo-1,2,3,4-tetrahydro-pyrimidine-5-carboxylic acid 2-(1,3-dimethyl-1H-thieno[2,3-c]pyrazol-5-yl)-ethyl ester (267);

4-fluoromethyl-6-methyl-2-thioxo-1,2,3,4-tetrahydro-pyrimidine-5-carboxylic acid 2-(2-bromo-thiophen-3-yl)-ethyl ester (268);

4-fluoromethyl-6-methyl-2-thioxo-1,2,3,4-tetrahydro-pyrimidine-5-carboxylic acid 3-thiophen-3-yl-allyl ester (269);

4-fluoromethyl-6-methyl-2-thioxo-1,2,3,4-tetrahydro-pyrimidine-5-carboxylic acid 3-furan-2-yl-propyl ester (270);

4-fluoromethyl-2-thioxo-1,2,3,4-tetrahydro-pyrimidine-5-carboxylic acid 2-thiophen-3-yl-ethyl ester (271);

4-benzyloxymethyl-6-methyl-2-thioxo-1,2,3,4-tetrahydro-pyrimidine-5-carboxylic acid 2-thiophen-3-yl-ethyl ester (272);

6-methyl-2-thioxo-1,2,3,4-tetrahydro-pyrimidine-5-carboxylic acid 2-(2-ethoxy-phenyl)-ethyl ester (273);

4-fluoromethyl-6-methyl-2-thioxo-1,2,3,4-tetrahydro-pyrimidine-5-carboxylic acid 2-(2-ethoxy-phenyl)-ethyl ester (274);

4-fluoromethyl-6-methyl-2-thioxo-1,2,3,4-tetrahydro-pyrimidine-5-carboxylic acid 2-(2-fluoro-thiophen-3-yl)-ethyl ester (1′);

6-methyl-2-thioxo-1,2,3,4-tetrahydro-pyrimidine-5-carboxylic acid 2-cyclopropyl-ethyl ester (2′);

6-methyl-2-thioxo-1,2,3,4-tetrahydro-pyrimidine-5-carboxylic acid cyclohex-3-enylmethyl ester (3′);

6-methyl-4-phenyl-2-thioxo-1,2,3,4-tetrahydro-pyrimidine-5-carboxylic acid 2-thiophen-3-yl-ethyl ester (4′);

6-methyl-2-thioxo-1,2,3,4-tetrahydro-pyrimidine-5-carboxylic acid 2-(3-methyl-thieno[2,3-c]isothiazol-5-yl)-ethyl ester (5′);

6-methyl-2-thioxo-1,2,3,4-tetrahydro-pyrimidine-4,5-dicarboxylic acid 4-ethyl ester 5-(2-thiophen-3-yl-ethyl) ester (6′);

8-methyl-6-thioxo-5,7-diaza-spiro[3,5]non-8-en-9-carboxylic acid 2-thiophen-3-yl-ethyl ester (7′);

6-methyl-2-thioxo-4-trifluoromethyl-1,2,3,4-tetrahydro-pyrimidine-5-carboxylic acid 2-thiophen-3-yl-ethyl ester (8′);

6-methyl-2-thioxo-1,2,3,4-tetrahydro-pyrimidine-5-carboxylic acid 3-thiophen-2-yl-propyl ester (9′);

4-fluoromethyl-6-methyl-2-thioxo-1,2,3,4-tetrahydro-pyrimidine-5-carboxylic acid 3-thiophen-2-yl-propyl ester (10′);

6-methyl-2-thioxo-1,2,3,4-tetrahydro-pyrimidine-5-carboxylic acid 5,6-dihydro-4H-cyclopenta[c]thiophen-5-yl ester (11′);

6-methyl-2-thioxo-1,2,3,4-tetrahydro-pyrimidine-5-carboxylic acid 1,3-dichloro-5,6-dihydro-4H-cyclopenta[c]thiophen-4-yl ester (12′);

7-methyl-5-thioxo-4,6-diaza-spiro[2.5]oct-7-ene-8-airboxylic acid 2-thiophen-3-yl-ethyl ester (13′);

6-methyl-2-thioxo-1,2,3,4-tetrahydro-pyrimidine-5-carboxylic acid 2-(3-nitro-phenyl)-ethyl ester (14′);

4-fluoromethyl-6-methyl-2-thioxo-1,2,3,4-tetrahydro-pyrimidine-5-carboxylic acid 3-chloro-4-methyl-thiophen-2-ylmethyl ester (15′);

4-fluoromethyl-2-thioxo-1,2,3,4-tetrahydro-pyrimidine-5-carboxylic acid 3-thiophen-2-yl-propyl ester (16′);

6-methyl-2-thioxo-1,2,3,4-tetrahydro-pyrimidine-5-carboxylic acid 5-carboxy-pentyl ester (18′);

6-methyl-2-thioxo-1,2,3,4-tetrahydro-pyrimidine-5-carboxylic acid 5-octylcarbamoyl-pentyl ester (19′);

6-methyl-2-thioxo-1,2,3,4-tetrahydro-pyrimidine-5-carboxylic acid 9-carboxy-nonyl ester (20′);

6-methyl-2-thioxo-1,2,3,4-tetrahydro-pyrimidine-5-carboxylic acid 1-chloro-5,6-dihydro-4H-cyclopenta[c]thiophen-4-yl ester (21′);

6-methyl-2-thioxo-1,2,3,4-tetrahydro-pyrimidine-5-carboxylic acid 2-(5-ethyl-furan-2-yl)-ethyl ester (22′);

4-fluoromethyl-6-methyl-2-thioxo-1,2,3,4-tetrahydro-pyrimidine-5-carboxylic acid 2-(5-ethyl-furan-2-yl)-ethyl ester (23′);

(R)-4-fluoromethyl-6-methyl-2-thioxo-1,2,3,4-tetrahydro-pyrimidine-5-carboxylic acid 2-thiophen-3-yl-ethyl ester (24′);

(S)-4-fluoromethyl-6-methyl-2-thioxo-1,2,3,4-tetrahydro-pyrimidine-5-carboxylic acid 2-thiophen-3-yl-ethyl ester (25′);

6-methyl-2-thioxo-1,2,3,4-tetrahydro-pyrimidine-5-carboxylic acid 2-(5-nitro-thiophen-3-yl)-ethyl ester (26′);

4-fluoromethyl-6-methyl-2-thioxo-1,2,3,4-tetrahydro-pyrimidine-5-carboxylic acid 2-(2-bromo-thiophen-3-yl)-1-methyl-ethyl ester (27′);

4-fluoromethyl-6-methyl-2-thioxo-1,2,3,4-tetrahydro-pyrimidine-5-carboxylic acid 2-(5-nitro-thiophen-3-yl)-ethyl ester (28′);

9-methyl-7-thioxo-6,8-diaza-spiro[4.5]dec-9-ene-10-carboxylic acid 2-thiophen-3-yl-ethyl ester (29′);

6-methyl-2-thioxo-1,2,3,4-tetrahydro-pyrimidine-5-carboxylic acid 5-methyl-[1,2,4]oxadiazol-3-ylmethyl ester (30′);

4-fluoromethyl-6-methyl-2-thioxo-1,2,3,4-tetrahydro-pyrimidine-5-carboxylic acid 3-phenyl-propyl ester (31′);

4-fluoromethyl-6-methyl-2-thioxo-1,2,3,4-tetrahydro-pyrimidine-5-carboxylic acid 2-(4-fluoro-phenyl)-ethyl ester (32′);

4-fluoromethyl-6-methyl-2-thioxo-1,2,3,4-tetrahydro-pyrimidine-5-carboxylic acid phenethyl ester (33′);

4-fluoromethyl-6-methyl-2-thioxo-1,2,3,4-tetrahydro-pyrimidine-5-carboxylic acid 4-chloro-benzyl ester (34′);

4-fluoromethyl-6-methyl-2-thioxo-1,2,3,4-tetrahydro-pyrimidine-5-carboxylic acid 3,5-dimethyl-benzyl ester (35′);

4-fluoromethyl-6-methyl-2-thioxo-1,2,3,4-tetrahydro-pyrimidine-5-carboxylic acid 5-chloro-2-methoxy-benzyl ester (36′);

4-fluoromethyl-6-methyl-2-thioxo-1,2,3,4-tetrahydro-pyrimidine-5-carboxylic acid 2-trifluoromethyl-benzyl ester (37′);

6-methyl-2-thioxo-1,2,3,4-tetrahydro-pyrimidine-5-carbothioic acid S-furan-2-ylmethyl ester (38′);

4-fluoromethyl-6-methyl-2-thioxo-1,2,3,4-tetrahydro-pyrimidine-5-carbothioic acid S-furan-2-ylmethyl ester (39′);

6-methyl-2-thioxo-1,2,3,4-tetrahydro-pyrimidine-5-carboxylic acid 2-(4-methoxy-thiophen-3-yl)-ethyl ester (40′);

4-fluoromethyl-6-methyl-2-thioxo-1,2,3,4-tetrahydro-pyrimidine-5-carboxylic acid 2-(4-methoxy-thiophen-3-yl)-ethyl ester (41′);

4-fluoromethyl-6-methyl-2-thioxo-1,2,3,4-tetrahydro-pyrimidine-5-carboxylic acid 2-(2-methoxy-phenyl)-ethyl ester (42′);

4-fluoromethyl-6-methyl-2-thioxo-1,2,3,4-tetrahydro-pyrimidine-5-carboxylic acid 3-(4-methoxy-phenyl)-propyl ester (43′);

4-fluoromethyl-6-methyl-2-thioxo-1,2,3,4-tetrahydro-pyrimidine-5-carboxylic acid cyclohex-3-enylmethyl ester (44′);

4-fluoromethyl-6-methyl-2-thioxo-1,2,3,4-tetrahydro-pyrimidine-5-carboxylic acid 3-phenyl-prop-2-inyl ester (45′);

4-fluoromethyl-6-methyl-2-thioxo-1,2,3,4-tetrahydro-pyrimidine-5-carboxylic acid 2-(benzoxazol-2-ylsulfanyl)-ethyl ester (46′);

6-methyl-2-thioxo-1,2,3,4-tetrahydro-pyrimidine-5-carboxylic acid 5-methyl-isoxazol-3-ylmethyl ester (47′);

4-fluoromethyl-6-methyl-2-thioxo-1,2,3,4-tetrahydro-pyrimidine-5-carboxylic acid 3-(2-oxo-benzothiazol-3-yl)-propyl ester (48′);

4-fluoromethyl-6-methyl-2-thioxo-1,2,3,4-tetrahydro-pyrimidine-5-carboxylic acid 3-(benzoxazol-2-ylsulfinyl)-propyl ester (49′);

4-(3H-imidazol-4-yl)-6-methyl-2-thioxo-1,2,3,4-tetrahydro-pyrimidine-5-carboxylic acid 2-thiophen-3-yl-ethyl ester (50′);

4-fluoromethyl-6-methyl-2-thioxo-1,2,3,4-tetrahydro-pyrimidine-5-carboxylic acid cyclohexylmethyl ester (51′);

4-fluoromethyl-6-methyl-2-thioxo-1,2,3,4-tetrahydro-pyrimidine-5-carboxylic acid 3-(2-fluoro-thiophen-3-yl)-propyl ester (52′);

4-fluoromethyl-6-methyl-2-thioxo-1,2,3,4-tetrahydro-pyrimidine-5-carboxylic acid 3-thiophen-3-yl-propyl ester (53′);

6-methyl-2-thioxo-1,2,3,4-tetrahydro-pyrimidine-5-carboxylic acid 2-(2-thiophen-3-yl-ethoxy)-ethyl ester (54′);

4-fluoromethyl-6-methyl-2-thioxo-1,2,3,4-tetrahydro-pyrimidine-5-carboxylic acid 4-(benzothiazol-2-ylsulfanyl)-butyl ester (55′);

4-fluoromethyl-6-methyl-2-thioxo-1,2,3,4-tetrahydro-pyrimidine-5-carboxylic acid 3-(4-fluorophenyl)-propyl ester (56′);

6-fluoromethyl-2-thioxo-1,2,3,4-tetrahydro-pyrimidine-5-carboxylic acid 2-thiophen-3-yl-ethyl ester (57′);

4-fluoromethyl-6-methyl-2-thioxo-1,2,3,4-tetrahydro-pyrimidine-5-carboxylic acid 3-benzo[b]thiophen-2-yl-propyl ester (58′);

4-fluoromethyl-6-methyl-2-thioxo-1,2,3,4-tetrahydro-pyrimidine-5-carboxylic acid 3-{4-[(2-methoxy-ethyl)-methyl-amino]-phenyl}-propyl ester (59′);

4-fluoromethyl-6-methyl-2-thioxo-1,2,3,4-tetrahydro-pyrimidine-5-carboxylic acid 2-pentafluorophenyl-ethyl ester (60′);

4-fluoromethyl-6-methyl-2-thioxo-1,2,3,4-tetrahydro-pyrimidine-5-carboxylic acid 4-thiophen-3-yl-butyl ester (61′);

4-fluoromethyl-6-methyl-2-thioxo-1,2,3,4-tetrahydro-pyrimidine-5-carboxylic acid [2,2′]bithiophenyl-5-ylmethyl ester (62′);

4-fluoromethyl-6-methyl-2-thioxo-1,2,3,4-tetrahydro-pyrimidine-5-carboxylic acid 3-methylsulfanyl-propyl ester (63′);

4-fluoromethyl-6-methyl-2-thioxo-1,2,3,4-tetrahydro-pyrimidine-5-carboxylic acid 1-(4-methoxy-phenyl)-cyclopentylmethyl ester (64′);

4-fluoromethyl-6-methyl-2-thioxo-1,2,3,4-tetrahydro-pyrimidine-5-carboxylic acid 2-(2,3,5,6-tetrafluoro-phenyl)-ethyl ester (65′);

4-fluoromethyl-6-methyl-2-thioxo-1,2,3,4-tetrahydro-pyrimidine-5-carboxylic acid 1-(4-methoxy-phenyl)-cyclopropylmethyl ester (66′);

6-hydroxymethyl-2-thioxo-1,2,3,4-tetrahydro-pyrimidine-5-carboxylic acid 2-thiophen-3-yl-ethyl ester (67′);

6-methyl-4-(2-methyl-propane-2-sulfonylmethyl)-2-thioxo-1,2,3,4-tetrahydro-pyrimidine-5-carboxylic acid 3-thiophen-3-yl-propyl ester (68′);

4-fluoromethyl-6-methyl-2-thioxo-1,2,3,4-tetrahydro-pyrimidine-5-carboxylic acid 3,5-difluoro-benzyl ester (69′);

4-fluoromethyl-6-methyl-2-thioxo-1,2,3,4-tetrahydro-pyrimidine-5-carboxylic acid 2-(3-chloro-phenyl)-ethyl ester (70′);

6-methyl-2-thioxo-1,2,3,4-tetrahydro-pyrimidine-5-carboxylic acid 2,2-difluoro-2-thiophen-3-yl-ethyl ester (71′);

4-fluoromethyl-6-methyl-2-thioxo-1,2,3,4-tetrahydro-pyrimidine-5-carboxylic acid 3-trifluoromethylsulfanyl-benzyl ester (72′);

4-fluoromethyl-6-methyl-2-thioxo-1,2,3,4-tetrahydro-pyrimidine-5-carboxylic acid 1-methyl-3-thiophen-2-yl-propyl ester (74′);

4-fluoromethyl-6-methyl-2-thioxo-1,2,3,4-tetrahydro-pyrimidine-5-carboxylic acid 2-(2,6-difluoro-phenyl)-ethyl ester (75′);

4-chloromethyl-6-methyl-2-thioxo-1,2,3,4-tetrahydro-pyrimidine-5-carboxylic acid 2-thiophen-3-yl-ethyl ester (76′);

4-fluoromethyl-6-methyl-2-thioxo-1,2,3,4-tetrahydro-pyrimidine-5-carboxylic acid 2-(2-nitro-phenyl)-ethyl ester (77′);

4-fluoromethyl-6-methyl-2-thioxo-1,2,3,4-tetrahydro-pyrimidine-5-carboxylic acid 5-chloro-2-nitro-benzyl ester (78′);

4-fluoromethyl-6-methyl-2-thioxo-1,2,3,4-tetrahydro-pyrimidine-5-carboxylic acid 2-chloro-4-fluoro-benzyl ester (79′);

4-difluoromethyl-6-methyl-2-thioxo-1,2,3,4-tetrahydro-pyrimidine-5-carboxylic acid 2-thiophen-3-yl-ethyl ester (80′);

4-fluoromethyl-6-methyl-2-thioxo-1,2,3,4-tetrahydro-pyrimidine-5-carboxylic acid 5-chloro-2-methoxy-benzyl ester (81′);

4-fluoromethyl-6-methyl-2-thioxo-1,2,3,4-tetrahydro-pyrimidine-5-carboxylic acid 3-chloro-4-methoxy-benzyl ester (82′);

4-fluoromethyl-6-methyl-2-thioxo-1,2,3,4-tetrahydro-pyrimidine-5-carboxylic acid 2-chloro-5-nitro-benzyl ester (83′);

4-fluoromethyl-6-methyl-2-thioxo-1,2,3,4-tetrahydro-pyrimidine-5-carboxylic acid 2-(5-chloro-2-methoxy-phenyl)-ethyl ester (84′);

4-fluoromethyl-6-methyl-2-thioxo-1,2,3,4-tetrahydro-pyrimidine-5-carboxylic acid 2-(3,5-difluoro-phenyl)-ethyl ester (85′);

4-fluoromethyl-6-methyl-2-thioxo-1,2,3,4-tetrahydro-pyrimidine-5-carboxylic acid 2-(2-chloro-4-fluoro-phenyl)-ethyl ester (86′);

4-fluoromethyl-6-methyl-2-thioxo-1,2,3,4-tetrahydro-pyrimidine-5-carboxylic acid 2-(2-chloro-6-fluoro-phenyl)-ethyl ester (87′);

4-fluoromethyl-6-methyl-2-thioxo-1,2,3,4-tetrahydro-pyrimidine-5-carbothioic acid S-phenethyl ester (88′);

4-fluoromethyl-6-methyl-2-thioxo-1,2,3,4-tetrahydro-pyrimidine-5-carboxylic acid 3,5-dichloro-benzyl ester (89′);

4-fluoromethyl-6-methyl-2-thioxo-1,2,3,4-tetrahydro-pyrimidine-5-carboxylic acid 2-(3,4-difluoro-phenyl)-ethyl ester (90′);

4-fluoromethyl-6-methyl-2-thioxo-1,2,3,4-tetrahydro-pyrimidine-5-carboxylic acid 3,4,5-trifluoro-benzyl ester (91′);

4-hydroxymethyl-6-methyl-2-thioxo-1,2,3,4-tetrahydro-pyrimidine-5-carboxylic acid 2-(5-chloro-thiophen-2-yl)-ethyl ester (1″);

4-methoxymethyl-6-methyl-2-thioxo-1,2,3,4-tetrahydro-pyrimidine-5-carboxylic acid 2-thiophen-3-yl-ethyl ester (2″);

4-hydroxymethyl-6-methyl-2-thioxo-1,2,3,4-tetrahydro-pyrimidine-5-carboxylic acid 2-thiophen-3-yl-ethyl ester (3″);

4-hydroxymethyl-6-methyl-2-thioxo-1,2,3,4-tetrahydro-pyrimidine-5-carboxylic acid 3-thiophen-2-yl-propyl ester (4″);

4-hydroxymethyl-6-methyl-2-thioxo-1,2,3,4-tetrahydro-pyrimidine-5-carboxylic acid 2-(4-fluoro-phenyl)-ethyl ester (5″);

4-hydroxymethyl-6-methyl-2-thioxo-1,2,3,4-tetrahydro-pyrimidine-5-carboxylic acid 3-thiophen-3-yl-propyl ester (6″);

4-hydroxymethyl-6-methyl-2-thioxo-1,2,3,4-tetrahydro-pyrimidine-5-carboxylic acid 2-(2-bromo-thiophen-3-yl)-1-methyl-ethyl ester (7″);

4-hydroxymethyl-6-methyl-2-thioxo-1,2,3,4-tetrahydro-pyrimidine-5-carboxylic acid benzyl ester (8″);

4-hydroxymethyl-6-methyl-2-thioxo-1,2,3,4-tetrahydro-pyrimidine-5-carboxylic acid 2-(2-methoxy-phenyl)-ethyl ester (9″);

4-hydroxymethyl-6-methyl-2-thioxo-1,2,3,4-tetrahydro-pyrimidine-5-carboxylic acid 2-(2-bromo-thiophen-3-yl)-ethyl ester (10″);

4-hydroxymethyl-6-methyl-2-thioxo-1,2,3,4-tetrahydro-pyrimidine-5-carboxylic acid 2-(2-ethoxy-phenyl)-ethyl ester (11″);

4-hydroxymethyl-6-methyl-2-thioxo-1,2,3,4-tetrahydro-pyrimidine-5-carboxylic acid 2-(4-dimethylamino-phenyl)-ethyl ester (12″);

4-hydroxymethyl-6-methyl-2-thioxo-1,2,3,4-tetrahydro-pyrimidine-5-carboxylic acid 3-(2-fluoro-thiophen-3-yl)-propyl ester (13″);

4-hydroxymethyl-6-methyl-2-thioxo-1,2,3,4-tetrahydro-pyrimidine-5-carboxylic acid 3-phenyl-propyl ester (14″);

4-(1-hydroxy-ethyl)-6-methyl-2-thioxo-1,2,3,4-tetrahydro-pyrimidine-5-carboxylic acid 3-thiophen-3-yl-propyl ester (15″);

4-hydroxymethyl-6-methyl-2-thioxo-1,2,3,4-tetrahydro-pyrimidine-5-carboxylic acid 2-(2-thiophen-3-yl-ethoxy)-ethyl ester (16″);

4-hydroxymethyl-6-methyl-2-thioxo-1,2,3,4-tetrahydro-pyrimidine-5-carboxylic acid 3-(3-methyl-thiophen-2-yl)-propyl ester (17″);

4-hydroxymethyl-6-methyl-2-thioxo-1,2,3,4-tetrahydro-pyrimidine-5-carboxylic acid 5-phenyl-pentyl ester (18″);

4-hydroxymethyl-6-methyl-2-thioxo-1,2,3,4-tetrahydro-pyrimidine-5-carboxylic acid 3-(5-ethyl-furan-2-yl)-propyl ester (19″);

4-ethylcarbamoyloxymethyl-6-methyl-2-thioxo-1,2,3,4-tetrahydro-pyrimidine-5-carboxylic acid 3-thiophen-2-yl-propyl ester (20″);

4-acetoxymethyl-6-methyl-2-thioxo-1,2,3,4-tetrahydro-pyrimidine-5-carboxylic acid 2-thiophen-3-yl-ethyl ester (21″);

4-acetoxymethyl-6-methyl-2-thioxo-1,2,3,4-tetrahydro-pyrimidine-5-carboxylic acid 3-thiophen-3-yl-propyl ester (22″);

4-acetoxymethyl-6-methyl-2-thioxo-1,2,3,4-tetrahydro-pyrimidine-5-carboxylic acid 3-thiophen-2-yl-propyl ester (23″);

4-hydroxymethyl-6-methyl-2-thioxo-1,2,3,4-tetrahydro-pyrimidine-5-carboxylic acid 3-naphthalen-1-yl-propyl ester (24″);

4-hydroxymethyl-6-methyl-2-thioxo-1,2,3,4-tetrahydro-pyrimidine-5-carboxylic acid 4-thiophen-3-yl-butyl ester (25″);

4-acetoxymethyl-6-methyl-2-thioxo-1,2,3,4-tetrahydro-pyrimidine-5-carboxylic acid 4-thiophen-3-yl-butyl ester (26″);

4-hydroxymethyl-6-methyl-2-thioxo-1,2,3,4-tetrahydro-pyrimidine-5-carboxylic acid 2-(4-benzyloxy-phenyl)-ethyl ester (27″);

4-hydroxymethyl-6-methyl-2-thioxo-1,2,3,4-tetrahydro-pyrimidine-5-carboxylic acid biphenyl-4-ylmethyl ester (28″);

4-hydroxymethyl-6-methyl-2-thioxo-1,2,3,4-tetrahydro-pyrimidine-5-carboxylic acid 3-methylsulfanyl-propyl ester (29″);

6-methyl-4-(2-methyl-propane-2-sulfonylmethyl)-2-thioxo-1,2,3,4-tetrahydro-pyrimidine-5-carboxylic acid 2-(2-ethoxy-phenyl)-ethyl ester (30″);

4-hydroxymethyl-6-methyl-2-thioxo-1,2,3,4-tetrahydro-pyrimidine-5-carboxylic acid 2-(3-chloro-phenyl)-ethyl ester (31″);

4-hydroxymethyl-6-methyl-2-thioxo-1,2,3,4-tetrahydro-pyrimidine-5-carboxylic acid 2-(3-nitro-phenyl)-ethyl ester (32″);

4-hydroxymethyl-6-methyl-2-thioxo-1,2,3,4-tetrahydro-pyrimidine-5-carboxylic acid 3-trifluoromethylsulfanyl-benzyl ester (33″);

4-hydroxymethyl-6-methyl-2-thioxo-1,2,3,4-tetrahydro-pyrimidine-5-carboxylic acid 3,5-dichloro-benzyl ester (34″);

4-hydroxymethyl-6-methyl-2-thioxo-1,2,3,4-tetrahydro-pyrimidine-5-carboxylic acid 2-(4-chloro-phenyl)-ethyl ester (35″);

4-hydroxymethyl-6-methyl-2-thioxo-1,2,3,4-tetrahydro-pyrimidine-5-carboxylic acid 2-cyclopentyl-ethyl ester (36″);

4-hydroxymethyl-6-methyl-2-thioxo-1,2,3,4-tetrahydro-pyrimidine-5-carboxylic acid 2-(3,5-dimethyl-phenyl)-ethyl ester (37″);

4-hydroxymethyl-6-methyl-2-thioxo-1,2,3,4-tetrahydro-pyrimidine-5-carboxylic acid 2-(3-trifluoromethyl-phenyl)-ethyl ester (38″);

4-hydroxymethyl-6-methyl-2-thioxo-1,2,3,4-tetrahydro-pyrimidine-5-carboxylic acid 2-(2,6-difluoro-phenyl)-ethyl ester (39″);

4-hydroxymethyl-6-methyl-2-thioxo-1,2,3,4-tetrahydro-pyrimidine-5-carboxylic acid 3-phenyl-prop-2-inyl ester (40″);

4-hydroxymethyl-6-methyl-2-thioxo-1,2,3,4-tetrahydro-pyrimidine-5-carboxylic acid 2-(2-chloro-phenyl)-ethyl ester (41″);

4-hydroxymethyl-6-methyl-2-thioxo-1,2,3,4-tetrahydro-pyrimidine-5-carboxylic acid 2-(3,4-dichloro-phenyl)-ethyl ester (42″);

4-hydroxymethyl-6-methyl-2-thioxo-1,2,3,4-tetrahydro-pyrimidine-5-carboxylic acid 2-(2,4-dichloro-phenyl)-ethyl ester (43″);

4-acetoxymethyl-6-methyl-2-thioxo-1,2,3,4-tetrahydro-pyrimidine-5-carboxylic acid 2-(3-chloro-phenyl)-ethyl ester (44″);

4-hydroxymethyl-6-methyl-2-thioxo-1,2,3,4-tetrahydro-pyrimidine-5-carboxylic acid 2-(4-methylsulfanyl-phenyl)-ethyl ester (45″);

4-hydroxymethyl-6-methyl-2-thioxo-1,2,3,4-tetrahydro-pyrimidine-5-carboxylic acid 3-phenoxy-benzyl ester (46″);

4-hydroxymethyl-6-methyl-2-thioxo-1,2,3,4-tetrahydro-pyrimidine-5-carboxylic acid 5-chloro-2-nitro-benzyl ester (47″);

4-hydroxymethyl-6-methyl-2-thioxo-1,2,3,4-tetrahydro-pyrimidine-5-carboxylic acid 2-chloro-4-fluoro-benzyl ester (48″);

4-ethoxycarbonyloxymethyl-6-methyl-2-thioxo-1,2,3,4-tetrahydro-pyrimidine-5-carboxylic acid 2-(3-chloro-phenyl)-ethyl ester (49″);

4-methanesulfonyloxymethyl-6-methyl-2-thioxo-1,2,3,4-tetrahydro-pyrimidine-5-carboxylic acid 3,5-dichloro-benzyl ester (50″);

4-hydroxymethyl-6-methyl-2-thioxo-1,2,3,4-tetrahydro-pyrimidine-5-carboxylic acid 3-trifluoromethyl-benzyl ester (51″);

4-hydroxymethyl-6-methyl-2-thioxo-1,2,3,4-tetrahydro-pyrimidine-5-carboxylic acid 1-(4-chloro-phenyl)-cyclopropylmethyl ester (52″);

4-hydroxymethyl-6-methyl-2-thioxo-1,2,3,4-tetrahydro-pyrimidine-5-carboxylic acid 2-(2,4,6-trifluoro-phenyl)-ethyl ester (53″);

4-hydroxymethyl-6-methyl-2-thioxo-1,2,3,4-tetrahydro-pyrimidine-5-carboxylic acid 2-(5-chloro-2-nitro-phenyl)-ethyl ester (54″);

4-hydroxymethyl-6-methyl-2-thioxo-1,2,3,4-tetrahydro-pyrimidine-5-carboxylic acid 2-(3-fluoro-4-methyl-phenyl)-ethyl ester (55″);

4-hydroxymethyl-6-methyl-2-thioxo-1,2,3,4-tetrahydro-pyrimidine-5-carboxylic acid 2-(5-chloro-2-methoxy-phenyl)-ethyl ester (56″);

4-hydroxymethyl-6-methyl-2-thioxo-1,2,3,4-tetrahydro-pyrimidine-5-carboxylic acid 2-(3,5-difluoro-phenyl)-ethyl ester (57″);

4-hydroxymethyl-6-methyl-2-thioxo-1,2,3,4-tetrahydro-pyrimidine-5-carboxylic acid 2-(2-chloro-4-fluoro-phenyl)-ethyl ester (58″);

4-hydroxymethyl-6-methyl-2-thioxo-1,2,3,4-tetrahydro-pyrimidine-5-carboxylic acid 2-(2-chloro-6-fluoro-phenyl)-ethyl ester (59″);

4-hydroxymethyl-6-methyl-2-thioxo-1,2,3,4-tetrahydro-pyrimidine-5-carboxylic acid 2-(4-ethoxy-phenyl)-ethyl ester (60″);

4-hydroxymethyl-6-methyl-2-thioxo-1,2,3,4-tetrahydro-pyrimidine-5-carboxylic acid 1-(2,4-dichloro-phenyl)-cyclopropylmethyl ester (61″);

4-hydroxymethyl-6-methyl-2-thioxo-1,2,3,4-tetrahydro-pyrimidine-5-carbothioic acid S-phenethyl ester (62″);

4-hydroxymethyl-6-methyl-2-thioxo-1,2,3,4-tetrahydro-pyrimidine-5-carboxylic acid 2-p-tolyl-ethyl ester (63″);

4-hydroxymethyl-6-methyl-2-thioxo-1,2,3,4-tetrahydro-pyrimidine-5-carboxylic acid 2-m-tolyl-ethyl ester (64″);

4-hydroxymethyl-6-methyl-2-thioxo-1,2,3,4-tetrahydro-pyrimidine-5-carboxylic acid 2-(5-ethyl-furan-2-yl)-ethyl ester (65″);

4-hydroxymethyl-6-methyl-2-thioxo-1,2,3,4-tetrahydro-pyrimidine-5-carboxylic acid 2-(4-fluoro-3-methyl-phenyl)-ethyl ester (66″);

4-hydroxymethyl-6-methyl-2-thioxo-1,2,3,4-tetrahydro-pyrimidine-5-carboxylic acid 2-(4-chloro-3-trifluoromethyl-phenyl)-ethyl ester (67″);

4-hydroxymethyl-6-methyl-2-thioxo-1,2,3,4-tetrahydro-pyrimidine-5-carboxylic acid 2-(3,4-difluoro-phenyl)-ethyl ester (68″);

4-hydroxymethyl-6-methyl-2-thioxo-1,2,3,4-tetrahydro-pyrimidine-5-carboxylic acid 2-(4-methoxy-3-methyl-phenyl)-ethyl ester (69″);

4-hydroxymethyl-6-methyl-2-thioxo-1,2,3,4-tetrahydro-pyrimidine-5-carboxylic acid 4-(benzothiazol-2-ylsulfanyl)-butyl ester (70″);

4-hydroxymethyl-6-methyl-2-thioxo-1,2,3,4-tetrahydro-pyrimidine-5-carboxylic acid 3,5-difluoro-benzyl ester (71″);

4-hydroxymethyl-6-methyl-2-thioxo-1,2,3,4-tetrahydro-pyrimidine-5-carboxylic acid 2-(4-ethyl-3-fluoro-phenyl)-ethyl ester (72″);

4-(2-amino-propionyloxymethyl)-6-methyl-2-thioxo-1,2,3,4-tetrahydro-pyrimidine-5-carboxylic acid 2-(3-trifluoromethyl-phenyl)-ethyl ester (73″);

4-hydroxymethyl-6-methyl-2-thioxo-1,2,3,4-tetrahydro-pyrimidine-5-carboxylic acid 2-(3-chloro-4-methyl-phenyl)-ethyl ester (74″);

4-hydroxymethyl-6-methyl-2-thioxo-1,2,3,4-tetrahydro-pyrimidine-5-carboxylic acid 2-(3-trifluoromethoxy-phenyl)-ethyl ester (75″);

4-(2-benzoyloxy-ethoxycarbonyloxymethyl)-6-methyl-2-thioxo-1,2,3,4-tetrahydro-pyrimidine-5-carboxylic acid 2-(3-chloro-phenyl)-ethyl ester (76″);

4-(2,2-dimethyl-[1,3]dioxolan-4-ylmethoxycarbonyloxymethyl)-6-methyl-2-thioxo-1,2,3,4-tetrahydro-pyrimidine-5-carboxylic acid 2-(3-nitro-phenyl)-ethyl ester (77″);

6-methyl-4-(5-methyl-2-oxo-[1,3]dioxol-4-ylmethoxycarbonyloxymethyl)-2-thioxo-1,2,3,4-tetrahydro-pyrimidine-5-carboxylic acid 2-(3-chloro-phenyl)-ethyl ester (78″);

4-(2-acetylsulfanyl-ethoxycarbonyloxymethyl)-6-methyl-2-thioxo-1,2,3,4-tetrahydro-pyrimidine-5-carboxylic acid 2-(3-nitro-phenyl)-ethyl ester (79″);

4-(2-methoxycarbonylnamino-ethoxycarbonyloxymethyl)-6-methyl-2-thioxo-1,2,3,4-tetrahydro-pyrimidine-5-carboxylic acid 2-(3-chloro-phenyl)-ethyl ester (80″);

4-[3-(2-acetoxy-phenyl)-propionyloxymethyl]-6-methyl-2-thioxo-1,2,3,4-tetrahydro-pyrimidine-5-carboxylic acid 2-(3-nitro-phenyl)-ethyl ester (81″);

4-(2-methoxycarbonyl-2-methyl-propoxycarbonyloxymethyl)-6-methyl-2-thioxo-1,2,3,4-tetrahydro-pyrimidine-5-carboxylic acid 2-(3-chloro-phenyl)-ethyl ester (82″);

4-ethoxycarbonylmethoxycarbonyloxymethyl-6-methyl-2-thioxo-1,2,3,4-tetrahydro-pyrimidine-5-carboxylic acid 2-(3-nitro-phenyl)-ethyl ester (83″);

4-(2-methanesulfonyl-ethoxycarbonyloxymethyl)-6-methyl-2-thioxo-1,2,3,4-tetrahydro-pyrimidine-5-carboxylic acid 2-(3-chloro-phenyl)-ethyl ester (84″);

4-(2-acetylamino-ethylcarbanoyloxymethyl)-6-methyl-2-thioxo-1,2,3,4-tetrahydro-pyrimidine-5-carboxylic acid 2-(3-nitro-phenyl)-ethyl ester (85″);

4-(2-dimethylamino-ethylcarbamoyloxymethyl)-6-methyl-2-thioxo-1,2,3,4-tetrahydro-pyrimidine-5-carboxylic acid 2-(3-chloro-phenyl)-ethyl ester trifluoroacetate (86″);

4-(2-acetoxy-ethylcarbanoyloxymethyl)-6-methyl-2-thioxo-1,2,3,4-tetrahydro-pyrimidine-5-carboxylic acid 2-(3-nitro-phenyl)-ethyl ester (87″);

6-methyl-4-(2-oxo-[1,3]dioxolan-4-ylmethoxycarbonyloxymethyl)-2-thioxo-1,2,3,4-tetrahydro-pyrimidine-5-carboxylic acid 2-(3-chloro-phenyl)-ethyl ester (88″);

4-benzoyloxymethyl-6-methyl-2-thioxo-1,2,3,4-tetrahydro-pyrimidine-5-carboxylic acid 2-(3-nitro-phenyl)-ethyl ester (89″);

terephthalic acid mono-{6-methyl-5-[2-(3-nitro-phenyl)-ethoxycarbonyl]-2-thioxo-1,2,3,4-tetrahydro-pyrimidin-4-ylmethyl}ester (90″);

4-Hydroxymethyl-6-methyl-2-thioxo-1,2,3,4-tetrahydro-pyrimidine-5-carboxylic acid 2-(4-diethylamino-3-nitro-phenyl)-ethyl ester (91″);

4-Hydroxymethyl-6-methyl-2-thioxo-1,2,3,4-tetrahydro-pyrimidine-5-carboxylic acid 2-[4-(ethyl-propylamino)-3-nitro-phenyl]-ethyl ester (92″);

4-Hydroxymethyl-6-methyl-2-thioxo-1,2,3,4-tetrahydro-pyrimidine-5-carboxylic acid 2-(4-dipropylamino-3-nitro-phenyl)-ethyl ester (93″);

4-Hydroxymethyl-6-methyl-2-thioxo-1,2,3,4-tetrahydro-pyrimidine-5-carboxylic acid 2-[4-(cyclopropyl-ethyl-amino)-3-nitro-phenyl]-ethyl ester (94″);

4-Hydroxymethyl-6-methyl-2-thioxo-1,2,3,4-tetrahydro-pyrimidine-5-carboxylic acid 2-[4-(cyclopropyl-propyl-amino)-3-nitro-phenyl]-ethyl ester (95″);

4-Hydroxymethyl-6-methyl-2-thioxo-1,2,3,4-tetrahydro-pyrimidine-5-carboxylic acid 2-[4-(allyl-methyl-amino)-3-nitro-phenyl]-ethyl ester (96″);

4-Hydroxymethyl-6-methyl-2-thioxo-1,2,3,4-tetrahydro-pyrimidine-5-carboxylic acid 2-[4-(allyl-ethyl-amino)-3-nitro-phenyl]-ethyl ester (97″);

4-Hydroxymethyl-6-methyl-2-thioxo-1,2,3,4-tetrahydro-pyrimidine-5-carboxylic acid 2-[4-(cyclopropylmethyl-methyl-amino)-3-nitro-phenyl]-ethyl ester (98″);

4-Hydroxymethyl-6-methyl-2-thioxo-1,2,3,4-tetrahydro-pyrimidine-5-carboxylic acid 2-(3-nitro-4-pyrrolidin-1-yl-phenyl)-ethyl ester (99″);

4-Hydroxymethyl-6-methyl-2-thioxo-1,2,3,4-tetrahydro-pyrimidine-5-carboxylic acid 2-(3-nitro-4-piperidin-1-yl-phenyl)-ethyl ester (100″); and

4-Hydroxymethyl-6-methyl-2-thioxo-1,2,3,4-tetrahydro-pyrimidine-5-carboxylic acid 2-[4-(2,5-dihydro-pyrrol-1-yl)-3-nitro-phenyl]-ethyl ester (101″).

Also, the compound of formula (1) according to the present invention can form a pharmaceutically acceptable salt. Such salt includes a non-toxic acid addition salt containing pharmaceutically acceptable anion, for example a salt with inorganic acids such as hydrochloric acid, sulfuric acid, nitric acid, phosphoric acid, hydrobromic acid, hydriodic acid, etc., a salt with organic carboxylic acids such as tartaric acid, formic acid, citric acid, acetic acid, trichloroacetic acid, trifluoroacetic acid, gluconic acid, benzoic acid, lactic acid, fumaric acid, maleic acid, etc., or a salt with sulfonic acids such as methanesulfonic acid, benzenesulfonic acid, p-toluenesulfonic acid, naphthalenesulfonic acid, etc.

Since the compound of formula (1) according to the present invention may have an asymmetric carbon atom when R² and R³ are different from each other, they can be present in the form of R or S enantiomer, or mixtures thereof including racemate. Thus, the present invention also includes all of these stereoisomers and their mixtures.

Further, the compound of formula (1) of the present invention may exist in a tautomer form as represented by the following formula (1a):

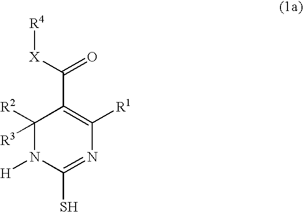

When the R² and R³ groups at 4-position of the tetrahydropyrimidine ring have different substituents from each other, the compound of formula (1) can have an asymmetric center, and so can exist as R or S isomer, racemite, mixtures of diastereomers, or the respective diastereomer. Thus, all these isomers are covered by the scope of the present invention.

By referring to known methods in Singh. H et al., Tetrahedron 1999, 55, 12873-12880; Kappe C. O, et al, Acc. Chem. Res. 2000, 33, 879; J. Comb. Chem. 2001, 3, 624, the new compound of formula (1) provided by the present invention can be prepared by a process characterized in that

(a) an oxazinane of the following formula (2):

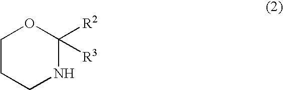

in which R² and R³ are defined as previously described, an acetoacetate of the following formula (3):

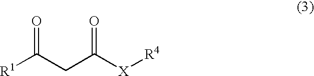

in which X, R¹, and R⁴ are defined as previously described, and a thiourea of the following formula (4a):

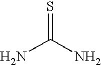

are reacted, or

(b) a carbonyl compound of the following formula (5):

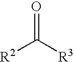

in which R² and R³ are defined as previously described, the acetoacetate of formula (3), and an isothiourea hydrochloride of the following formula (4b):

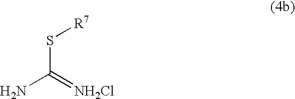

in which R⁷ is aralkyl or alkoxycarbonyl, each of which is unsubstituted or substituted by alkoxy, are reacted. Therefore, it is another object of the present invention to provide such a process.

The compound of formula (1) prepared according to the process of the present invention may be converted to its salt form through conventional methods, and also may be separated or purified by such conventional workup processes as chromatography, recrystallization, etc.

Further, as stated above, the compound of formula (1) of the present invention is effective for treating or preventing hepatitis C, and so it is another object of the present invention to provide a composition for the treatment or prevention of hepatitis C, comprising as an active ingredient the compound of formula (1), or its pharmaceutically acceptable salt, hydrate, solvete, or isomer including tautomer, together with pharmaceutically acceptable carrier.

The present invention also provides a use of the compound of formula (1) for the treatment or prevention of hepatitis C, and a method for the treatment or prevention of hepatitis C by administering the compound of formula (1) to a patient suffering from hepatitis C.

Below, in determining the effect of the compound of formula (1) for the treatment or prevention of hepatitis C, experimental theories and specific protocols will be explained.

HCV is a virus having 9.4 kb positive RNA as a genome, from which one polyprotein is produced. This polyprotein is subjected to processing to give proteins of core, envelope1, envelope2, NS2, NS3, NS4A, NS4B, NS5A, and NS5B. The major factors in the RNA genome are the internal ribosome binding site (IRES) including 5′UTR (UnTranslated Region), and 3′UTR. The major targets of most antiviral agents for these proteins and RNA include IRES NS3 coding protease and RNA helicase, NS5B coding RNA-dependent RNA polymerase, etc.

In order to screen and evaluate the anti-HCV therapeutic agents in enzyme level, full-length, truncated, or tethered three enzymes obtained by overexpressing and purifying NS3, a protease of HCV, in bacteria using the bacterial expression vector are used. This process may be briefly explained as follows.

A drug is serially diluted and mixed with each enzyme having a constant concentration. Then, a certain amount of a polypeptide consisting of 20 amino acids which is formed by tagging Fam at the N-terminal and Tamra at the C-terminal of a substrate having a part of HCV protease target sequence is added and the resulting mixture is reacted. The substrate is decomposed to develop fluorescence when a drug does not exist, but the drug inhibits the decomposition to reduce the fluorescence. This pattern is directly proportional to the initial velocity Vo of the enzyme, and so the fluorescence measured is considered as the Vo value. According to this method, Vo values are measured for each concentration of the drug. The % value of each Vo on the basis of control containing no drug is calculated, and the concentration when the % value is 50 is determined to be IC50.

In order to, screen and evaluate the anti-HCV therapeutic agents in cell level, the following experiments are carried out using a replicon cell line, Rep5.1 [Lohmann, et al., Replication of subgenomic hepatitis C virus RNAs in a hepatoma cell line. Science 285:110-113 (1999)].

Rep5.1 cells, the replicon cell line, are cultured in DMEM-10 containing 500 μg/ml of G418. The Rep5.1 cells in log phase are suspended by trypsin treatment, and 5000 cells are distributed to each well of a 96-well plate. After 4 hours when the cells are attached to the plate, the serially diluted drug is added to each well. After 48 hours, a part of the treated cells are observed by microscope, and MTT assay is carried out to evaluate the drug toxicity. The cytotoxicity is estimated in percentage based on the control containing no drug. After 72 hours, the remaining cells are fixed by methanol/acetone (50/50, v/v), and the decrease of HCV proteins is measured by using HCV antibody. The extent of decrease is estimated in percentage based on the control containing no drug, and the concentration of drug at which 50% of proteins is decreased is determined to be IC50. The compounds showing activity via this procedure are validated by Western blotting. In order to find out whether the decrease of HCV protein is related to the decrease of replicon RNA, Rep5.1 cell is treated with some selected drugs for 72 hours, RNA is purified, dot blotted, and hybridized with isotope labelled nucleic acid to determine the amounts of actin mRNA and replicon RNA, and finally the amount of RNA is compared.

When the compound of the present invention, of which therapeutic and preventive effect for hepatitis C is confirmed according to the above procedure, is used for clinical purpose, a preferable amount to be administered ranges from 1 to 1000 mg per kg of body weight a day. The total daily dosage nay be administered once or over several times. However, a specific administration dosage for an individual patient can be varied depending on specific compound used, body weight, sex or hygienic condition of the patient, diet, time or method of administration, excretion rate, mixing ratio of agent, severity of disease to be treated, etc.

The compound of the present invention may be administered via any pathways, but is preferable in the form of injection or oral preparation.

A preparation for injection, for example, sterilized aqueous or oily suspension for injection, can be prepared according to a known procedure using suitable dispersing agent, wetting agent, or suspending agent. Solvents which can be used for the preparation for injection include water, Ringer's fluid, and isotonic NaCl solution, and also sterilized fixing oil nay be conveniently used as the solvent or suspending media. Any non-stimulative fixing oil including mono-, di-glyceride nay be used for this purpose. Fatty acid such as oleic acid nay also be used such preparation for injection.

As the solid preparation for oral administration, there are capsules, tablets, pills, powders and granules, etc., and capsules and tablets are particularly useful. It is also desirable for tablets and pills to be formulated into enteric-coated preparation. The form of solid preparation may be prepared by mixing the active compound of formula (1) of the present invention with at least one carrier selected from a group consisting of inactive diluents such as sucrose, lactose, starch, etc., lubricants such as magnesium stearate, disintegrating agent, and binding agent.

When the compound of the present invention is clinically applied for obtaining the desired therapeutic and preventive effect for hepatitis C, the active compound of formula (1) can be administered alone or in combination with immunomodulators such as α-interferon, β-interferon, or γ-interferon; antiviral agents such as ribavirin or amantadine; inhibitors against hepatitis C protease; inhibitors against helicase, polymerase, metaloprotease, or other targets in the life cycle of hepatitis C virus including inflow of the internal ribosome; or their mixtures.

The above processes according to the present invention will be more specifically explained in the following examples. However, it should be understood that these examples are not in any manner intended to limit the scope of the present invention.

### EXAMPLES

### Example 1

### Synthesis of 6-methyl-2-thioxo-1,2,3,4-tetrahydro-pyrimidine-5-carboxylic acid benzyl ester (6)

Benzyl acetoacetate (1.92 g, 10 mmol), thiourea (0.98 g, 13 mmol), and oxazinane (1.22 g, 14 mmol) were introduced to a round bottomed flask, and anhydrous acetonitrile (30 ml) was added thereto. To this solution was added trifluoroacetic acid (1.54 ml, 20 mmol), which was then stirred at reflux for 6 hours. After completion of the reaction, acetonitrile was distilled under reduced pressure. To the filtrate were added water (30 ml) and ethyl acetate (50 ml), and then the organic layer was separated, dried, and purified by column chromatography (n-hexne:ethyl acetate=3:1) to give the title compound (2.09 g) in the yield of 80%. When the purity of the title compound is low, it was further purified by preparative HPLC.

¹H NMR(CDCl₃): δ 7.49(s, 1H), 7.40-7.33(m, 5H), 6.82(s, 1H), 5.18(s, 2H), 4.18(s, 2H), 2.30(s, 3H)

MS(M+1): 263

### Example 2

### Synthesis of 6-methyl-2-thioxo-1,2,3,4-tetrahydro-pyrimidine-5-carboxylic acid 4-nitro-benzyl ester (7)

4-Nitrobenzyl alcohol (1.53 g, 10 mmol) was introduced into a round bottomed flask, and dichloromethane (10 ml) was added thereto. After the solution was cooled using an ice bath, diketene (1.26 g, 15 mmol) was slowly added, and triethylamine (0.14 ml, 1 mmol) was added. This solution was warmed to room temperature, stirred for 3 hours, and water (10 mg) was added. The organic layer was dried over MgSO₄ and distilled under reduced pressure to give 4-nitrobenzyl acetoacetate (2.30 g, Yield=97%). The resulting 4-nitrobenzyl acetoacetate was reacted according to the same procedure as Example 1 to give the title compound in the yield of 68% (2.09 g).

¹H NMR(CDCl₃): δ 8.25(d, 2H), 7.53(s, 1H), 7.51(d, 2H), 6.57(s, 1H), 5.27(s, 2H), 4.21(s, 2H), 2.54(s, 3H)

MS(M+1): 308

### Example 3

### Synthesis of 6-methyl-2-thioxo-1,2,3,4-tetrahydro-pyrimidine-5-carboxylic acid 4-methoxy-benzyl ester (8)

4-Methoxybenzyl alcohol was reacted according to the sane procedure as Example 2 to give the title compound in the yield of 70% (2.04 g).

¹H NMR(CDCl₃): δ 7.47(s, 1H), 7.29(d, 2H), 6.90(d, 2H), 6.80(s, 1H), 5.10(s, 2H), 4.14(s, 2H), 3.81(s, 3H), 2.28(s, 3H)

MS(M+1): 293

Alcohols and diketenes were reacted according to the sane procedure as Example 2, and the resulting acetoacetates were reacted with thioureas, and alkyated or functionalized oxazinanes according to the same procedure as Example 1 to give the following compounds.

First, preparations of alkylated or functionalized oxazinanes are described below.

### Preparation 1

### Synthesis of N,O-dimethylfluoroacetamide

Sodium salt of fluoroacetic acid (10.0 g, 100 mmol) was mixed with dichloromethane (100 ml). N,O-dimethylhydroxylanine hydrochloride (11.7 g, 120 mmol), EDC (23.0 g, 120 mmol), diisopropylethylamine (34.8 ml, 200 mmol), and dimethylaninopyridine (1.22 g, 10.0 mmol) were added in the order thereto, and the mixture was stirred at room temperature for 12 hours. After completion of the reaction, the reaction solution was poured to saturated ammonium chloride solution (100 ml), and the organic layer was separated by separatory funnel. The remaining aqueous layer was extracted with dichloromethane (100 ml). The organic layers were combined, dried over MgSO₄, concentrated under reduced pressure, and purified by silica gel column chromatography (hexane/EtOAc=1/1, v/v) to give the title compound (10.9 g, Yield 90.0%).

¹H-NMR (400 MHz, CDCl): δ 5.14 (s, 1H), 5.02 (s, 1H), 3.70 (s, 3H), 3.22 (s, 3H)

### Preparation 2

### Synthesis of 2-fluoromethyl-[1,3]-oxazinane

The compound prepared in Preparation 1 (2.85 g, 23.5 mmol) was dissolved in THF (50 ml) and cooled to −78° C. DIBAL (1.0 M hexane solution, 35.3 ml, 35.3 mmol) was slowly added thereto, and the mixture was stirred for 1 hour at −78° C . The reaction solution was warmed to 0° C., 1N aqueous hydrochloric acid solution (10 ml) was slowly added thereto, and the mixture was stirred for 30 minutes. To this solution were added 3-amino-1-propanol (3.60 ml, 47.1 mmol) and benzene (50 ml), and the mixture was then heated at reflux. After 10 hours when the reaction was completed, the reaction solution was poured to saturated sodium hydrogen carbonate solution (100 ml), and the organic layer was separated by separatory funnel. The remaining aqueous layer was extracted with diethylether (50 ml □5). The organic layers were combined, dried over MgSO, concentrated under reduced pressure, and purified by silica gel column chromatography (hexane/Et₂=½, v/v) to give the title compound (935 mg, Yield 33.3%).

¹H-NMR (400 MHz, CDCl): δ4.48-4.40 (m, 1H), 4.38-4.28 (m, 2H), 4.21-4.13 (m, 1H), 3.86-3.76 (m ,H), 3.27-3.18 (m, 1H), 2.97 (t, 1H), 1.87-1.71 (m, 1H), 170 (br s, 1H), 1.39 (d, 1H)

### 6-Methyl-2-thioxo-1,2,3,4-tetrahydro-pyrimidine-5-carboxylic acid benzol[1.3]dioxol-5-yl methyl ester (9)

¹H NMR(CDCl₃): δ 7.20(s, 1H), 6.83-6.78(m, 3H), 6.52(s, 1H), 5.97(s, 2H), 5.09(s, 2H), 4.16(s, 2H), 2.29(s, 3H)

MS(M+1): 307

### 6-Methyl-2-thioxo-1,2,3,4-tetrahydro-pyrimidine-5-carboxylic acid naphthalen-1-ylmethyl ester (10)

¹H NMR(CDCl₃): δ 8.00-7.45(m, 7H), 7.22(s, 1H), 6.51(s, 1H), 5.68(s, 2H), 4.14(s, 2H), 2.26(s, 3H)

MS(M+1): 313

### 6-Methyl-2-thioxo-1,2,3,4-tetrahydro-pyrimidine-5-carboxylic acid 4-methoxycarbonyl-benzyl ester (11)

¹H NMR(CDCl₃): δ 8.07(d, 2H), 7.97(s, 1H), 7.45(d, 2H), 5.30(s, 2H), 3.93(s, 3H), 3.86(s, 2H), 2.51(s, 3H)

MS(M+1): 321

### 6-Methyl-2-thioxo-1,2,3,4-tetrahydro-pyrimidine-5-carboxylic acid furan-2-ylmethyl ester (12)

¹H NMR(CDCl₃): δ 7.42(s, 1H), 7.28(s, 1H), 6.61(s, 1H), 6.41-6.36(m, 2H), 4.14(s, 2H), 2.28(s, 3H)

MS(M+1): 253

### 6-Methyl-2-thioxo-1,2,3,4-tetrahydro-pyrimidine-5-carboxylic acid 3-pyridin-4-yl-propyl ester (13)

¹H NMR(DMSO-d₆): δ 8.74(d, 2H), 7.83(d, 2H), 4.20(t, 2H), 3.87(s, 2H), 2.90(t, 2H), 2.48(s, 3H), 2.02(m, 2H)

MS(M+1): 291

### 6-Methyl-2-thioxo-1,2,3,4-tetrahydro-pyrimidine-5-carboxylic acid 3-phenyl-propyl ester (14)

¹H NMR(CDCl₃): δ 7.50(s, 1H), 7.32-7.17(m, 5H), 6.80(s, 1H), 4.17(t, 2H), 4.08(s, 2H), 2.70(t, 2H), 2.29(s, 3H), 2.00(m, 2H)

MS(M+1): 307

### 6-Methyl-2-thioxo-1,2,3,4-tetrahydro-pyrimidine-5-carboxylic acid 2-benzyloxy-ethyl ester (15)

¹H NMR(CDCl₃): δ 7.38(s, 1H), 7.37-7.30(m, 5H), 6.82(s, 1H), 4.56(s, 2H), 4.32(t, 2H), 4.15(s, 2H), 3.70(t, 2H), 2.28(s, 3H)

MS(M+1): 292

### 6-Methyl-2-thioxo-1,2,3,4-tetrahydro-pyrimidine-5-carboxylic acid allyl ester (16)

¹H NMR(DMSO-d₆): δ 10.15(s, 1H), 9.14(s, 1H), 6.13-6.04(m, 1H), 5.45-5.34(m, 2H), 4.72(d, 2H), 4.07(s, 2H), 2.34(s, 3H)

MS(M+1): 213

### 6-Naphthalen-1-yl-2-thioxo-1,2,3,4-tetrahydro-pyrimidine-5-carboxylic acid benzyl ester (17)

¹H NMR(DMSO-d₆): δ 10.30(s, 1H), 9.16(s, 1H), 7.95-7.91(m, 2H), 7.78(d, 1H), 7.55-7.35(m, 5H), 7.18-7.10(m, 4H), 6.64(d, 2H), 4.79(s, 2H)

MS(M+1): 375

### 6-Benzyl-2-thioxo-1,2,3,4-tetrahydro-pyrimidine-5-carboxylic acid benzyl ester (18)

¹H NMR(DMSO-d₆): δ 10.11(s, 1H), 9.02(s, 1H), 7.35-7.27(m, 10H), 5.12(s, 2H), 4.03(s, 2H), 3.99(d, 2H)

MS(M+1): 339

### 6-Phenethyl-2-thioxo-1,2,3,4-tetrahydro-pyrimidine-5-carboxylic acid benzyl ester (19)

¹H NMR(DMSO-d₆): δ 10.00(s, 1H), 8.79(s, 1H), 7.18-7.14(m, 5H), 7.06-6.93(m, 5H), 4.94(s, 2H), 3.74(s, 2H), 2.66-2.62(m, 2H), 2.53-2.48(m, 2H)

MS(M+1): 353

6-Methyl-2-thioxo-1,2,3,4-tetrahydro-pyrimidine-5-carboxylic acid biphenyl-4-ylmethyl ester (22)

¹H NMR(CDCl₃): δ 7.61-7.37(m, 9H), 7.27(s, 1H), 6.62(s, 1H), 5.22(s, 2H), 4.20(s, 2H), 2.31(s, 3H)

MS(M+1): 339

### 6-Methyl-2-thioxo-1,2,3,4-tetrahydro-pyrimidine-5-carboxylic acid (R)-1-phenyl-ethyl ester (23)

¹H NMR(CDCl₃): δ 7.48(s, 1H), 7.38-7.27(m, 5H), 6.83(s, 1H), 5.95(q, 2H), 4.19(s, 2H), 2.29(s, 3H), 1.60(d, 3H)

MS(M+1): 277

### 6-Methyl-2-thioxo-1,2,3,4-tetrahydro-pyrimidine-5-carboxylic acid

### 2-(4-methyl-thiazol-5-yl)-ethyl ester (24)

¹H NMR(DMSO-d₆): δ 8.61(s, 1H), 6.63(s, 1H), 4.34(t, 2H), 4.14(s, 2H), 3.15(t, 2H), 2.42(s, 3H), 2.26(s, 1H)

MS(M+1): 298

### 6-Propyl-2-thioxo-1,2,3,4-tetrahydro-pyrmidine-5-carboxylic acid benzyl ester (25)

¹H NMR(DMSO-d₆): δ 9.97(s,1H), 8.95(s, 1H), 7.39-7.32(m, 5H), 5.10(s, 2H), 3.90(s, 2H), 2.55(t, 2H), 1.47-1.41(m, 2H), 0.81(t, 3H)

MS(M+1): 291

6-Methyl-2-thioxo-1,2,3,4-tetrahydro-pyrimidine-5-carboxylic acid (R,S)-1-phenyl-ethyl ester (26)

¹H NMR(CDCl₃): δ 7.48(s, 1H), 7.38-7.27(m, 5H), 6.83(s, 1H), 5.95(q, 2H), 4.19(s, 2H), 2.29(s, 3H), 1.60(d, 3H)

MS(M+1): 277

### 6-Methyl-2-thioxo-1,2,3,4-tetrahydro-pyrimidine-5-carboxylic acid 1-methyl-1-phenyl-ethyl ester (27)

¹H NMR(CDCl₃): δ 7.45(s, 1H), 7.35-7.27(m, 5H), 6.60(s, 1H), 4.21(s, 2H), 2.24(s, 3H), 1.80(s, 6H)

MS(M+1): 291

### 6-Methyl-2-thioxo-1,2,3,4-tetrahydro-pyrimidine-5-carboxylic acid cyclo-hexylmethyl ester (29)

¹H NMR(CDCl₃): δ 7.17(s, 1H), 6.54(s, 1H), 4.17(s, 2H), 3.39(d, 2H), 2.29(s, 3H), 1.76-1.61(m, 6H), 1.31-0.94(m, 5H)

MS(M+1): 269

### 6-Methyl-2-thioxo-1,2,3,4-tetrahydro-pyrimidine-5-carboxylic acid hexyl ester (30)

¹H NMR(CDCl₃): δ 7.39(s, 1H), 6.68(s, 1H), 4.18(s, 2H), 2.29(s, 3H), 1.67(m, 2H), 1.30(m, 6H), 0.90(m, 3H)

MS(M+1): 257

### 6-Methyl-2-thioxo-1,2,3,4-tetrahydro-pyrimidine-5-carboxylic acid 8-phenyl-octyl ester (33)

¹H NMR(CDCl₃): δ 7.36(s, 1H), 7.30-7.17(m, 5H), 6.82(s, 1H), 4.14(s, 2H), 4.12(t, 2H), 2.62(t, 2H), 2.28(s, 3H), 1.65(m, 4H), 1.32(m, 8H)

MS(M+1): 361

### 6-Methyl-2-thioxo-1,2,3,4-tetrahydro-pyrimidine-5-carboxylic acid phenethyl ester (34)

¹H NMR(CDCl₃): δ 7.33-7.20(m, 5H), 6.57(s, 1H), 4.37(t, 2H), 4.09(s, 2H), 2.97(t, 2H), 2.21(s, 3H)

MS(M+1): 277

### 6-Methyl-2-thioxo-1,2,3,4-tetrahydro-pyrimidine-5-carboxylic acid 1-methyl-heptyl ester (35)

¹H NMR(CDCl₃): δ 7.20(s, 1H), 6.57(s, 1H), 4.98(m, 1H), 4.15(s, 2H), 2.30(s, 3H), 1.49-1.23(m, 13H), 0.90(t, 3H)

MS(M+1): 285

### 6-Methyl-2-thioxo-1,2,3,4-tetrahydro-pyrimidine-5-carboxylic acid 3-(3,4-dimethoxy-phenyl)-propyl ester (36)

¹H NMR(CDCl₃): δ 7.25(s, 1H), 6.81-6.72(m, 3H), 6.69(s, 1H), 4.17(t, 2H), 4.13(s, 2H), 3.88(d, 6H), 2.66(t, 2H), 2.29(s, 3H), 1.98(t, 2H)

MS(M+1): 351

6-Methyl-2-thioxo-1,2,3,4-tetrahydro-pyrimidine-5-carboxylic acid cyclohexyl ester (37)

¹H NMR(CDCl₃): δ 7.22(s, 1H), 6.59(s, 1H), 4.88(m, 1H), 4.14(s, 2H), 2.29(s, 3H), 2.28-1.69(m, 4H), 1.45-1.29(m, 6H)

MS(M+1): 255

### 6-Methyl-2-thioxo-1,2,3,4-tetrahydro-pyrimidine-5-carboxylic acid 1-methyl-pentyl ester (39)

¹H NMR(CDCl₃): δ 7.25(s, 1H), 6.63(s, 1H), 4.96(m, 1H), 4.14(s, 2H), 2.29(s, 3H), 1.54-1.24(m, 9H), 0.90(t, 3H)

MS(M+1): 257

### 6-Methyl-2-thioxo-1,2,3,4-tetrahydro-pyrimidine-5-carboxylic acid 3,4-dimethoxy-benzyl ester (40)

¹H NMR(CDCl₃): δ 7.25(s, 1H), 6.94-6.84(m, 3H), 6.59(s, 1H), 5.11(s, 2H), 4.11(s, 2H), 3.89(s, 6H), 2.29(s, 3H)

MS(M+1): 323

6-Methyl-2-thioxo-1,2,3,4-tetrahydro-pyrimidine-5-carboxylic acid 2,4-dichloro-benzyl ester (41)

¹H NMR(CDCl₃): δ 7.44-7.32(m, 3H), 7.25(s, 1H), 5.24(s, 2H), 4.19(s, 2H), 2.29(s, 2H)

MS(M+1): 332

### 6-Methyl-2-thioxo-1,2,3,4-tetrahydro-pyrimidine-5-carboxylic acid 1-methyl-3-phenyl-propyl ester (42)

¹H NMR(CDCl₃): δ 7.39(s, 1H), 7.32-7.16(m, 5H), 6.73(s, 1H), 5.04(m, 1H), 4.14(q, 2H), 2.67(m, 2H), 2.30(s, 3H), 1.97-1.85(m, 2H), 1.66(s, 3H)

MS(M+1): 305

### 6-Methyl-2-thioxo-1,2,3,4-tetrahydro-pyrimidine-5-carboxylic acid 4-chloro-benzyl ester (43)

¹H NMR(CDCl₃): δ 7.36(d, 2H), 7.30(d, 2H), 6.50(s, 1H), 5.13(s, 2H), 4.17(s, 2H), 2.29(s, 2H)

MS(M+1): 297

### 2-Thioxo-1,2,3,4-tetrahydro-pyrimidine-5-carboxylic acid benzyl ester (44)

¹H NMR(DMSO-d₆): δ 10.02 (s, 1H), 8.99 (s, 1H), 7.40-7.25 (m, 5H), 6.94 (d, 1H), 5.13 (s, 2H), 3.95 (s, 2H)

MS(M+1): 249

### 6-Methyl-2-thioxo-1,2,3,4-tetrahydro-pyrimidine-5-carboxylic acid 4-t-butyl-benzyl ester (45)

¹H NMR(DMSO-d₆): δ 10.01 (s, 1H), 8.99 (s, 1H), 7.39 (d, 2H), 7.28 (d, 2H), 5.06 (s, 2H), 3.90 (s, 2H), 2.19 (s, 3H), 1.27 (s, 9H)

MS(M+1): 319

### 6-Methyl-2-thioxo-1,2,3,4-tetrahydro-pyrimidine-5-carboxylic acid cyclo-propylmethyl ester (46)

¹H NMR(CDCl₃): δ 7.15(s, 1H), 6.65(s, 1H), 4.18(s, 2H), 3.99(d, 2H), 2.30(s, 3H), 1.53(m, 1H), 0.60(m, 2H), 0.30(m, 2H)

MS(M+1): 227

### 6-Methyl-2-thioxo-1,2,3,4-tetrahydro-pyrimidine-5-carboxylic acid-ethyl-hexyl ester (47)

¹H NMR(CDCl₃): δ 7.36(s, 1H), 6.73(s, 1H), 4.91(m, 1H), 4.15(s, 2H), 2.30 (s, 3H),1.66(m, 2H), 1.33(m, 6H), 0.90(m, 6H)

MS(M+1): 285

### 6-Methyl-2-thioxo-1,2,3,4-tetrahydro-pyrimidine-5-carboxylic acid 3-cyano-benzyl ester (48)

¹H NMR(CDCl₃): δ 7.64-7.57(m, 3H), 6.60(s, 1H), 5.19(s, 2H), 4.19(s, 2H), 2.30(s, 3H)

MS(M+1): 288

### 6-Methyl-2-thioxo-1,2,3,4-tetrahydro-pyrimidine-5-carboxylic acid 4-benzyloxy-benzyl ester (49)

¹H NMR(CDCl₃): δ 7.44-7.29(m, 7H), 7.22(s, 1H), 6.98(d, 2H), 6.55(s, 1H), 5.11(s, 2H), 5.08(s, 2H), 4.15(s, 2H), 2.28(s, 3H)

MS(M+1): 369

### 6-Methyl-2-thioxo-1,2,3,4-tetrahydro-pyrimidine-5-carboxylic acid 3-benzyloxy-benzyl ester (50)

¹H NMR(CDCl₃): δ 7.44-7.30(m, 6H), 7.27(s, 1H), 6.95(m, 3H), 6.72(s, 1H), 5.14(s, 2H), 5.08(s, 2H), 4.14(s, 2H), 2.28(s, 3H)

MS(M+1): 369

### 6-Methyl-2-thioxo-1,2,3,4-tetrahydro-pyrimidine-5-carboxylic acid 4-butyl-benzyl ester (51)

¹H NMR(DMSO-d₆): δ 10.00 (s, 1H), 8.97 (s, 1H), 7.24 (d, 2H), 7.18 (d, 2H), 5.06 (s, 2H), 3.90 (s, 2H), 2.56 (t, 2H), 2.18 (s, 3H), 1.53 (quintet, 2H), 1.29 (quintent, 2H), 0.88 (t, 3H)

MS(M+1): 319

### 6-Methyl-2-thioxo-1,2,3,4-tetrahydro-pyrimidine-5-carboxylic acid 4-methylsulfanyl-benzyl ester (52)

¹H NMR(DMSO-d₆): δ 10.00 (s, 1H), 8.98 (s, 1H), 7.29 (d, 2H), 7.25 (d, 2H), 5.06 (s, 2H), 3.90 (s, 2H), 2.46 (s, 3H), 2.20 (s, 3H)

MS(M+1): 309

### 6-Methyl-2-thioxo-1,2,3,4-tetrahydro-pyrimidine-5-carboxylic acid 3-methyl-but-2-enyl ester (53)

¹H NMR(DMSO-d₆): δ 9.96 (s, 1H), 8.96 (s, 1H), 5.30 (bt, 1H), 4.54 (d, 2H), 3.86 (s, 2H), 2.17 (s, 3H), 1.71 (s, 3H), 1.66 (s, 3H)

MS(M+1): 241

### 6-Methyl-2-thioxo-1,2,3,4-tetrahydro-pyrimidine-5-carboxylic acid 3-phenyl-allyl ester (54)

¹H NMR(DMSO-d₆): δ 10.01 (s, 1H), 9.00 (s, 1H), 7.46 (d, 2H), 7.34 (t, 2H), 7.27 (t, 1H), 6.66 (d, 1H), 6.39 (dt, 1H), 4.73 (d, 2H), 3.93 (s, 2H), 2.10 (s, 3H)

MS(M+1): 289

### 6-Methyl-2-thioxo-1,2,3,4-tetrahydro-pyrimidine-5-carboxylic acid 1-vinyl-but-3-enyl ester (55)

¹H NMR(DMSO-d₆): δ 9.98 (s, 1H), 8.99 (s, 1H), 5.90-5.67 (m, 2H), 5.30-5.00 (m, 5H), 3.90 (q, 2H), 2.45-2.30 (m, 2H), 2.17 (s, 3H)

MS(M+1): 253

### 6-Methyl-2-thioxo-1,2,3,4-tetrahydro-pyrimidine-5-carboxylic acid 3,7-dimethyl-octa-2,6-dienyl ester (56)

¹H NMR(DMSO-d₆): δ 9.96 (s, 1H), 8.96 (s, 1H), 5.29 (t, 1H), 5.05 (t, 1H), 4.55 (d, 2H), 3.86 (s, 2H), 2.17 (s, 3H), 2.10-1.95 (m, 4H), 1.65 (s, 3H), 1.63 (s, 3H), 1.55 (s, 3H)

MS(M+1): 309

### 6-Methyl-2-thioxo-1,2,3,4-tetrahydro-pyrimidine-5-carboxylic acid phenyl-o-tolyl-methyl ester (57)

¹H NMR(DMSO-d₆): δ 10.04 (s, 1H), 9.01 (s, 1H), 7.40-7.15 (m, 9H), 6.95 (s, 1H), 4.01 (s,2H), 2.26 (s, 3H), 2.18 (s, 3H)

MS(M+1): 353

### 6-Methyl-2-thioxo-1,2,3,4-tetrahydro-pyrimidine-5-carboxylic acid 4-benzyloxy-3-methoxy-benzyl ester (58)

¹H NMR(DMSO-d₆): δ 9.96(s, 1H), 8.93(s, 1H), 7.44-7.31(m, 5H), 7.01-6.99(m, 2H), 6.88-6.86(m, 1H), 5.07(s, 2H), 5.20(s, 2H), 3.90(s, 2H), 3.76(s, 3H), 2.19(s, 3H)

MS(M+1): 399

### 6-Methyl-2-thioxo-1,2,3,4-tetrahydro-pyrimidine-5-carboxylic acid 2-(ethyl-m-tolyl-amino)-ethyl ester (59)

¹H NMR(CDCl₃): δ 7.18-6.98(m, 4H), 4.37(t, 2H), 3.90(t, 2H), 3.66(s, 2H), 3.60(q, 2H), 2.31(s, 3H), 2.15(s, 2H) 1.14(t, 3H)

MS(M+1): 334

### 6-Methyl-2-thioxo-1,2,3,4-tetrahydro-pyrimidine-5-carboxylic acid 4-bromo-benzyl ester (60)

¹H NMR(CDCl₃): δ 7.52(d, 2H), 7.30(d, 2H), 5.12(s, 1H), 4.03(s, 2H), 2.24(s, 2H),

MS(M+1): 342

### 6-Methyl-2-thioxo-1,2,3,4-tetrahydro-pyrimidine-5-carboxylic acid 4-fluoro-benzyl ester (61)

¹H NMR(CDCl₃): δ 7.41(d, 2H), 7.10(d, 2H), 5.13(s, 2H), 4.02(s, 2H), 2.23(s, 2H)

MS(M+1): 281

### 6-Methoxy-2-thioxo-1,2,3,4-tetrahydro-pyrimidine-5-carboxylic acid benzyl ester (63)

¹H NMR(DMSO-d₆): δ 9.52(s, 1H), 9.05(s, 1H), 7.31-7.39(m, 5H), 5.09(s, 2H), 4.25(s, 2H), 3.47(s, 3H)

MS(M+1): 279

### 6-Methoxymethyl-2-thioxo-1,2,3,4-tetrahydro-pyrimidine-5-carboxylic acid benzyl ester (64)

¹H NMR(DMSO-d₆): δ 9.34(s, 1H), 9.11(s, 1H), 7.33-7.41(m, 5H), 5.13(s, 2H), 4.42(s, 2H), 3.97(s, 2H), 3.26(s, 3H)

MS(M+1): 293

### 6-Methyl-2-thioxo-1,2,3,4-tetrahydro-pyrimidine-5-carboxylic acid 2-methoxy-benzyl ester (65)

¹H NMR(CDCl₃): δ 7.35-7.29(m, 2H), 6.97-6.89(m, 2H), 6.63(s, 1H), 5.22(s, 2H), 4.17(s, 2H), 3.85(s, 3H), 2.29(s, 3H)

MS(M+1): 293

### 6-Methyl-2-thioxo-1,2,3,4-tetrahydro-pyrimidine-5-carboxylic acid 2-ethoxy-benzyl ester (66)

¹H NMR(CDCl₃): δ 7.31-7.29(m, 2H), 7.17(s, 1H), 6.95-6.87(m, 2H), 6.52(s, 1H), 5.24(s, 2H), 4.18(s, 2H), 4.09(q, 2H), 2.29(s, 3H), 1.27(t, 3H)

MS(M+1): 307

### 6-Methyl-2-thioxo-1,2,3,4-tetrahydro-pyrimidine-5-carboxylic acid cyclopentyl ester (67)

¹H NMR(CDCl₃): δ 7.40(s, 1H), 6.77(s, 1H), 5.23(m, 1H), 4.12(s, 2H), 2.31(s, 3H), 1.89(m, 2H), 1.71-1.59(m, 6H)

MS(M+1): 241

### 6-Methyl-2-thioxo-1,2,3,4-tetrahydro-pyrimidine-5-carboxylic acid cyclopentylmethyl ester (68)

¹H NMR(CDCl₃): δ 7.42(s, 1H), 6.77(s, 1H), 4.16(s, 2H), 4.04(d, 2H), 2.29(s, 3H), 2.17(m, 1H), 1.76(m, 2H), 1.62-1.26(m, 6H)

MS(M+1): 255

### 6-Methyl-2-thioxo-1,2,3,4-tetrahydro-pyrimidine-5-carboxylic acid 2,4-difluoro-benzyl ester (69)

¹H NMR(CDCl₃): δ 7.53-7.32(m, 1H), 7.25(s, 1H), 6.90(m, 2H), 5.18(s, 2H), 4.19(s, 2H), 2.28(s, 3H)

MS(M+1): 299

6-Methyl-2-thioxo-1,2,3,4-tetrahydro-pyrimidine-5-carboxylic acid

### 2-chloro-4-fluoro-benzyl ester (70)

¹H NMR(CDCl₃): δ 7.43(m, 1H), 7.29(s, 1H), 7.18(m, 1H), 7.02(m, 1H), 6.61(s, 1H), 5.21(s, 2H), 4.17(s, 2H), 2.29(s, 3H)

MS(M+1): 315

### 6-Methyl-2-thioxo-1,2,3,4-tetrahydro-pyrimidine-5-carboxylic acid 4-ethoxy-benzyl ester (71)

¹H NMR(CDCl₃): δ 7.28(d, 2H), 6.89(d, 2H), 6.87(s, 1H), 5.10(s, 2H), 4.15(s, 2H), 4.03(q, 2H), 2.29(s, 3H), 1.42(t, 3H)

MS(M+1): 307

6-Methyl-2-thioxo-1,2,3,4-tetrahydro-pyrimidine-5-carboxylic acid 4-isobutoxy-benzyl ester (72)

¹H NMR(CDCl₃): δ 7.27(d, 2H), 6.88(d, 2H), 6.63(s, 1H), 5.10(s, 2H), 4.15(s, 2H), 3.73(d, 2H), 2.28(s, 3H), 2.10(m, 1H), 1.04(d, 6H)

MS(M+1): 335

### 6-Methyl-2-thioxo-1,2,3,4-tetrahydro-pyrimidine-5-carboxylic acid 4-methanesulfonyl-benzyl ester (74)

¹H NMR(DMSO-d₆): δ 10.05 (s, 1H), 9.04 (s, 1H), 7.92 (d, 2H), 7.62 (d, 2H), 5.22 (s, 2H), 3.95 (s, 2H), 3.21 (s, 3H), 2.20 (s, 3H)

MS(M+1): 341

### 6-Methyl-2-thioxo-1,2,3,4-tetrahydro-pyrimidine-5-carboxylic acid 3-iodo-benzyl ester (75)

¹H NMR(CDCl₃): δ 7.69(m, 2H), 7.31(m, 2H), 7.13(m, 1H), 6.62(s, 1H), 5.10(s, 2H), 4.18(s, 2H), 2.30(s, 3H)

MS(M+1): 389

### 6-Methyl-2-thioxo-1,2,3,4-tetrahydro-pyrimidine-5-carboxylic acid 3,4-dimethyl-benzyl ester (76)

¹H NMR(CDCl₃): δ 7.19(s, 1H), 7.14-7.06(m, 3H), 6.54(s, 1H), 5.11(s, 2H), 4.16(s, 2H), 2.28(s, 3H), 2.29(s, 6H)

MS(M+1): 291

### 6-Methyl-2-thioxo-1,2,3,4-tetrahydro-pyrimidine-5-carboxylic acid 3-nitro-benzyl ester (77)

¹H NMR(CDCl₃): δ 8.22(m, 1H), 7.57(m, 1H), 7.02(m, 1H), 6.56(s, 1H), 5.26(s, 2H), 4.21(s, 2H), 2.31(s, 3H)

MS(M+1): 308

### 6-Methyl-2-thioxo-1,2,3,4-tetrahydro-pyrimidine-5-carboxylic acid 4-propoxy-benzyl ester (78)

¹H NMR(CDCl₃): δ 7.28(d, 2H), 6.88(d, 2H), 6.60(s, 1H), 5.10(s, 2H), 4.15(s, 2H), 3.92(t, 2H), 2.28(s, 3H), 1.82(m, 2H), 1.06(t, 3H)

MS(M+1): 321

### 6-Methyl-2-thioxo-1,2,3,4-tetrahydro-pyrimidine-5-carboxylic acid 4-dimethylamino-benzyl ester (79)

¹H NMR(CDCl₃): δ 7.33(s, 1H), 7.25-7.22(m, 1H), 6.70(m, 4H), 5.13(s, 2H), 4.18(s, 2H), 2.96(s, 6H), 2.30(s, 3H)

MS(M+1): 306

### 6-Methyl-2-thioxo-1,2,3,4-tetrahydro-pyrimidine-5-carboxylic acid cyclobutylmethyl ester (80)

¹H NMR(CDCl₃): δ 7.47(s, 1H), 6.82(s, 1H), 4.15(s, 2H), 4.12(d, 2H), 2.66(m, 1H), 2.29(s, 3H), 2.07(m, 2H), 1.93(m, 2H), 1.74(m, 2H)

MS(M+1): 241

### 6-Methyl-2-thioxo-1,2,3,4-tetrahydro-pyrimidine-5-carboxylic acid 3,5-dimethoxy-benzyl ester (81)

¹H NMR(CDCl₃): δ 6.70(s, 1H), 6.48-6.42(m, 3H), 5.11(s, 1H), 4.19(s, 2H), 3.80(s, 6H), 2.30(s, 3H)

MS(M+1): 323

### 6-Methyl-2-thioxo-1,2,3,4-tetrahydro-pyrimidine-5-carboxylic acid 3,5-dimethyl-benzyl ester (82)

¹H NMR(CDCl₃): δ 7.31(s, 1H), 6.98-6.95(m, 3H), 6.65(s, 1H), 5.10(s, 2H), 4.17(s, 2H), 2.35(s, 6H), 2.29(s, 3H)

MS(M+1): 291

### 6-Methyl-2-thioxo-1,2,3,4-tetrahydro-pyrimidine-5-carboxylic acid 3-fluoro-benzyl ester (83)

¹H NMR(CDCl₃): δ 7.37(m, 2H), 7.12-7.02(m, 3H), 6.67(s, 1H), 5.17(s, 2H), 4.19(s, 2H), 2.30(s, 3H)

MS(M+1): 281

### 6-Methyl-2-thioxo-1,2,3,4-tetrahydro-pyrimidine-5-carboxylic acid 2-chloro-benzyl ester (84)

¹H NMR(CDCl₃): δ 7.42-7.27(m, 4H), 7.26(s, 1H), 6.67(s, 1H), 5.27(s, 2H), 4.19(s, 2H), 2.30(s, 3H)

MS(M+1): 297

### 6-Methyl-2-thioxo-1,2,3,4-tetrahydro-pyrimidine-5-carbothioic acid S-benzyl ester (85)

¹H NMR(CDCl₃): δ 7.47(s, 1H), 7.31-7.26(m, 5H), 6.80(s, 1H), 4.26(s, 2H), 4.19(s, 2H), 2.30(s, 3H)

MS(M+1): 279

### 6-Methyl-2-thioxo-1,2,3,4-tetrahydro-pyrimidine-5-carboxylic acid 1-benzyl-piperidin-4-yl ester (86)

¹H NMR(CDCl₃): δ 7.72(s, 1H), 7.26-7.34(m, 5H), 7.09(s, 1H), 4.86-4.90(m, 1H), 4.12(s, 2H), 3.50(s, 2H), 2.63(bs, 2H), 2.30(bs, 2H), 2.27(s, 3H), 1.85-1.95(m, 2H), 1.67-1.74(m, 2H)

MS(M+1): 346

### 2-Thioxo-1,2,3,6-tetrahydro-pyrimidine-4,5-dicarboxylic acid dibenzyl ester (87)

¹H NMR(DMSO-d₆): δ 10.76(s, 2H), 9.14(s, 1H), 7.34(bs, 5H), 5.07(s, 2H), 4.99(s, 2H), 3.99(s, 2H)

MS(M+1): 383

### 6-Methyl-2-thioxo-1,2,3,4-tetrahydro-pyrimidine-5-carboxylic acid 3-(4-methoxy-phenyl)-propyl ester (88)

¹H NMR(CDCl₃): δ 7.35(s, 1H), 7.10(d, 2H), 6.85(d, 2H), 6.69(s, 1H), 4.17(t, 2H), 4.13(s. 2H), 3.79(s, 3H), 2.65(t, 2H), 2.30(s, 3H), 1.95(m, 2H)

MS(M+1): 321

### 6-Methyl-2-thioxo-1,2,3,4-tetrahydro-pyrimidine-5-carboxylic acid 4-methyl-benzyl ester (89)

¹H NMR(CDCl₃): δ 7.28-7.13(m, 4H), 6.58(s, 1H), 5.14(s, 2H), 4.17(s, 2H), 2.36(s, 3H), 2.29(s, 3H)

MS(M+1): 277

### 6-Methyl-2-thioxo-1,2,3,4-tetrahydro-pyrimidine-5-carboxylic acid 3-methyl-benzyl ester (90)

¹H NMR(CDCl₃): δ 7.45(s, 1H), 7.26-7.16(m, 4H), 6.79(s, 1H), 5.13(s, 2H), 4.16(s, 2H), 2.36(s, 3H), 2.29(s, 3H)

MS(M+1): 277

### 6-Methyl-2-thioxo-1,2,3,4-tetrahydro-pyrimidine-5-carboxylic acid 3-chloro-benzyl ester (91)

¹H NMR(CDCl₃): δ 7.42(s, 1H), 7.33-7.21(m, 4H), 6.74(s, 1H), 5.16(s, 2H), 4.19(s, 2H), 2.29(s, 3H)

MS(M+1): 297

### 6-Methyl-2-thioxo-1,2,3,4-tetrahydro-pyrimidine-5-carboxylic acid 2-(4-methoxy-phenyl)-ethyl ester (92)

¹H NMR(CDCl₃): δ 7.29(s, 1H), 7.13(d, 2H), 6.84(d, 2H), 6.64(s, 1H), 4.34(t, 2H), 4.09(s, 2H),3.80(s, 3H), 2.91(t, 2H), 2.26(s, 3H)

MS(M+1): 307

### 6-Methyl-2-thioxo-1,2,3,4-tetrahydro-pyrimidine-5-carboxylic acid 2-(4-fluoro-phenyl)-ethyl ester (93)

¹H NMR(CDCl_{'}): δ 7.36(s, 1H), 7.17(d, 2H), 6.97(d, 2H), 6.71(s, 1H), 4.35(t, 2H), 4.08(s, 2H), 2.95(t, 2H), 2.22(s, 3H)

MS(M+1): 295

### 6-Methyl-2-thioxo-1,2,3,4-tetrahydro-pyrimidine-5-carboxylic acid 2-(4-chloro-phenyl)-ethyl ester (94)

¹H NMR(CDCl₃): δ 7.52(s, 1H), 7.29(d, 2H), 7.12(d, 2H), 6.85(s, 1H), 4.36(t, 2H), 4.08(s, 2H), 2.93(t, 2H), 2.22(s, 3H)

MS(M+1): 311

### 6-Methyl-2-thioxo-1,2,3,4-tetrahydro-pyrimidine-5-carboxylic acid 2-(4-dimethylamino-phenyl)-ethyl ester (95)

¹H NMR(CDCl₃): δ 7.37(s, 1H), 7.08(d, 2H), 6.71(m, 3H), 4.32(t, 2H), 4.10(s, 2H), 2.93(s, 6H), 2.86(t, 2H), 2.23(s, 3H)

MS(M+1): 320

### 6-Methyl-2-thioxo-1,2,3,4-tetrahydro-pyrimidine-5-carboxylic acid (s)-(−)-4-isopropenyl-cyclohex-1-enylmethyl ester (96)

¹H NMR(DMSO-d₆): δ 9.97 (s, 1H), 8.96 (s, 1H), 5.60 (s, 1H), 4.61 (s, 2H), 4.44 (s, 2H), 3.88 (s, 2H), 2.18 (s, 3H), 2.15-1.75 (m, 6H), 1.70 (s, 3H), 1.45-1.35 (m, 1H)

MS(M+1): 307

### 6-Methyl-2-thioxo-1,2,3,4-tetrahydro-pyrimidine-5-carboxylic acid 6,6-dimethyl-bicyclo[3.1.1]hept-2-en-2-ylmethyl ester (97)

¹H NMR(DMSO-d₆): δ 9.97 (s, 1H), 8.96 (s, 1H), 5.54 (s, 1H), 4.43 (s, 2H), 3.87 (s, 2H), 2.40-2.00 (m, 8H), 1.26 (s, 3H), 1.09 (d, 1H), 0.78 (s, 3H)

MS(M+1): 307

### 6-Methyl-2-thioxo-1,2,3,4-tetrahydro-pyrimidine-5-carboxylic acid 2-chloro-allyl ester (98)

¹H NMR(DMSO-d₆): δ 10.08 (s, 1H), 9.04 (s, 1H), 5.62 (s, 1H), 5.48 (s, 1H), 4.71 (s, 2H), 3.92 (s, 2H), 2.17 (s, 3H)

MS(M+1): 247

### 6-Methyl-2-thioxo-1,2,3,4-tetrahydro-pyrimidine-5-carboxylic acid 3-bromo-but-3-enyl ester (99)

¹H NMR(DMSO-d₆): δ 9.99 (s, 1H), 8.99 (s, 1H), 5.82 (s, 1H), 5.53 (s, 1H), 4.20 (t, 2H), 3.87 (s, 2H), 2.76 (t, 2H), 2.17 (s, 3H)

MS(M+1): 306

### 6-Methyl-2-thioxo-1,2,3,4-tetrahydro-pyrimidine-5-carboxylic acid 2-trifluoromethyl-benzyl ester (100)

¹H NMR(DMSO-d₆): δ 10.05 (s, 1H), 9.00 (s, 1H), 7.80-7.50 (m, 4H), 5.26 (s, 2H), 3.91 (s, 2H), 2.76 (t, 2H), 2.18 (s, 3H)

MS(M+1): 331

### 6-Methyl-2-thioxo-1,2,3,4-tetrahydro-pyrimidine-5-carboxylic acid 3-trifluoromethyl-benzyl ester (101)

¹H NMR(DMSO-d₆): δ 10.04 (s, 1H), 9.00 (s, 1H), 7.75-7.60 (m, 4H), 5.20 (s, 2H), 3.93 (s, 2H), 2.76 (t, 2H), 2.19 (s, 3H)

MS(M+1): 331

### 6-Methyl-2-thioxo-1,2,3,4-tetrahydro-pyrimidine-5-carboxylic acid 2-(1H-indol-3-yl)-ethyl ester (102)

¹H NMR(DMSO-d₆): δ 10.85 (s, 1H), 9.95 (s, 1H), 8.97 (s, 1H), 7.53 (d, 1H), 7.33 (d, 1H), 7.16 (s, 1H), 7.06 (t, 1H), 6.98 (t, 1H), 4.27 (t, 2H), 3.86 (s, 2H), 3.00 (t, 2H), 2.13 (s, 3H)

MS(M+1): 316

### 6-Methyl-2-thioxo-1,2,3,4-tetrahydro-pyrimidine-5-carboxylic acid 3-(4-fluoro-phenyl)-propyl ester (103)

¹H NMR(DMSO-d₆): δ 9.96(s, 1H), 8.97(s, 1H), 7.24-7.21(m, 2H), 7.11-7.07(m, 2H), 4.01(t, 2H), 3.89(s, 2H), 2.64-2.62(t, 2H), 2.18(s, 3H), 1.90-1.84(m, 2H)

MS(M+1): 309

### 6-Methyl-2-thioxo-1,2,3,4-tetrahydro-pyrimidine-5-carboxylic acid 3-(2-methoxy-phenyl)-propyl ester (104)

¹H NMR(DMSO-d₆): δ 9.95(s, 1H), 8.97(s, 1H), 7.19-7.09(m, 2H), 6.94-6.83(m, 2H), 4.00(t, 2H), 3.88(s, 2H), 3.75(s, 3H), 2.59(t, 2H), 2.18(s, 3H), 1.85-1.79(m, 2H)

MS(M+1): 321

### 6-Methyl-2-thioxo-1,2,3,4-tetrahydro-pyrimidine-5-carboxylic acid 3-(4-methylsulfanyl-phenyl)-propyl ester (105)

¹H NMR(DMSO-d₆): δ 9.96(s, 1H), 8.95(s, 1H), 7.19-7.12(m, 4H), 4.00(t, 2H), 3.89(s, 2H), 2.59(t, 2H), 2.43(s, 3H), 2.17(s, 3H), 1.89-1.82(m, 2H)

MS(M+1): 337

### 6-Methyl-2-thioxo-1,2,3,4-tetrahydro-pyrimidine-5-carboxylic acid 3-(3-trifluoromethoxy-phenyl)-propyl ester (106)

¹H NMR(DMSO-d₆): δ 9.95(s, 1H), 8.96(s, 1H), 7.43-7.39(m, 1H), 7.25-7.16(m, 3H), 4.02(t, 2H), 3.86(s, 2H), 2.69(t, 2H), 2.17(s, 3H), 1.93-1.86(m, 2H)

MS(M+1): 375

### 6-Methyl-2-thioxo-1,2,3,4-tetrahydro-pyrimidine-5-carboxylic acid 3-(4-trifluoromethyl-phenyl)-propyl ester (107)

¹H NMR(DMSO-d₆): δ 9.95(s, 1H), 8.95(s, 1H), 7.09-7.07(m, 4H), 4.00(t, 2H), 3.88(s, 2H), 2.58(t, 2H), 2.45(s, 3H), 2.17(s, 3H), 1.89-1.82(m, 2H)

MS(M+1): 359

### 6-Methyl-2-thioxo-1,2,3,4-tetrahydro-pyrimidine-5-carboxylic acid 3-(4-benzyloxy-phenyl)-propyl ester (108)

¹H NMR(DMSO-d₆): δ 9.96(s, 1H), 8.96(s, 1H), 7.44-7.29(m, 5H), 7.09(d, 2H), 6.90(d, 2H), 5.05(s, 2H), 4.00(t, 2H), 3.89(s, 2H), 2.56(t, 2H), 2.17(s, 3H), 1.87-1.81(m, 2H)

MS(M+1): 397

### 4-Methoxymethyl-6-methyl-2-thioxo-1,2,3,4-tetrahydro-pyrimidine-5-carboxylic acid benzyl ester (109)

¹H NMR(CDCl₃): δ 7.65(s, 1H), 7.33-7.39(m, 5H), 7.15(s, 1H), 5.19(dd, 2H), 4.51-4.54(m, 1H), 3.33-3.36(m, 2H), 3.31(s, 3H), 2.30 (s, 3H)

MS(M+1): 307

### 6-Methyl-2-thioxo-1,2,3,4-tetrahydro-pyrimidine-5-carboxylic acid 2-methylsulfinyl-benzyl ester (110)

¹H NMR(DMSO-d₆): δ 10.01 (s, 1H), 8.97 (s, 1H), 7.35 (bt, 3H), 7.19 (bt, 1H), 5.10 (s, 2H), 3.90 (s, 2H), 2.48 (s, 3H), 2.18 (s, 3H)

MS(M+1): 309

### 6-Methyl-2-thioxo-1,2,3,4-tetrahydro-pyrimidine-5-carboxylic acid 2-isobutylsulfanyl-benzyl ester (111)

¹H NMR(DMSO-d₆): δ 10.01 (s, 1H), 8.98 (s, 1H), 7.43 (d, 1H), 7.40-7.35 (m, 2H), 7.20 (t, 1H), 5.15 (s, 2H), 3.90 (s, 2H), 2.85 (d, 2H), 2.18 (s, 3H), 1.78-1.72 (m, 1H), 0.96 (d, 6H)

MS(M+1): 351

### 6-Methyl-2-thioxo-1,2,3,4-tetrahydro-pyrimidine-5-carboxylic acid 2-methanesulfonyl-benzyl ester (112)

¹H NMR(DMSO-d₆): δ 10.06 (s, 1H), 9.03 (s, 1H), 7.97 (d, 1H), 7.75 (bt, 1H), 7.65-7.60 (m, 2H), 5.52 (s, 2H), 3.97 (s, 2H), 3.27 (s, 3H), 2.20 (s, 3H)

MS(M+1): 341

### 6-Methyl-2-thioxo-1,2,3,4-tetrahydro-pyrimidine-5-carboxylic acid 2-(2-methyl-propane-1-sulfonyl)-benzyl ester (113)

¹H NMR(DMSO-d₆): δ 10.07 (s, 1H), 9.05 (s, 1H), 7.94 (d, 1H), 7.76 (t, 1H), 7.62 (t, 2H), 5.50 (s, 2H), 3.97 (s, 2H), 3.25 (d, 2H), 2.20 (s, 3H), 2.12-2.05 (m, 1H), 0.97 (d, 6H)

MS(M+1): 383

### 6-Methyl-2-thioxo-1,2,3,4-tetrahydro-pyrimidine-5-carboxylic acid 4-trifluoromethyl-benzyl ester (114)

¹H NMR(DMSO-d₆): δ 10.04 (s, 1H), 9.02 (s, 1H), 7.73 (d, 2H), 7.52 (t, 2H), 5.18 (s, 2H), 3.95 (s, 2H), 3.25 (d, 2H), 2.20 (s, 3H)

MS(M+1): 331

### 6-Methyl-2-thioxo-1,2,3,4-tetrahydro-pyrimidine-5-carboxylic acid 2-(2-methoxy-phenyl)-ethyl ester (115)

¹H NMR(CDCl₃): δ 7.34(s, 1H), 7.23-6.85(m, 4H), 6.70(s, 1H), 4.36(t, 2H), 4.08(s, 2H), 3.83(s, 3H), 2.97(t, 2H), 2.22(s, 3H)

MS(M+1): 307

### 6-Methyl-2-thioxo-1,2,3,4-tetrahydro-pyrimidine-5-carboxylic acid 2-(2-fluoro-phenyl)-ethyl ester (116)

¹H NMR(CDCl₃): δ 7.40(s, 1H), 7.26-7.01(m, 4H), 6.74(s, 1H), 4.38(t, 2H), 4.07(s, 2H), 3.00(t, 3H), 2.21(s, 3H)

MS(M+1): 295

### 6-Methyl-2-thioxo-1,2,3,4-tetrahydro-pyrimidine-5-carboxylic acid

### 2-(3-chloro-phenyl)-ethyl ester (117)

¹H NMR(CDCl₃): δ 7.46(s, 1H), 7.26-7.07(m, 4H), 6.80(s, 1H), 4.36(t, 2H), 4.08(s, 2H), 2.95(t, 3H), 2.22(s, 3H)

MS(M+1): 311

### 6-Methyl-2-thioxo-1,2,3,4-tetrahydro-pyrimidine-5-carboxylic acid 2-(2-chloro-phenyl)-ethyl ester (118)

¹H NMR(CDCl₃): δ 7.48(s, 1H), 7.38-7.18(m, 4H), 6.81(s, 1H), 4.41(t, 2H), 4.08(s, 2H), 3.12(t, 3H), 2.22(s, 3H)

MS(M+1): 311

### 6-Methyl-2-thioxo-1,2,3,4-tetrahydro-pyrimidine-5-carboxylic acid 2-(4-nitro-phenyl)-ethyl ester (119)

¹H NMR(CDCl₃): δ 8.20(d, 2H), 7.39(d, 2H), 7.21(s, 4H), 6.57(s, 1H), 4.34(t, 2H), 4.07(s, 2H), 3.08(t, 3H), 2.22(s, 3H)

MS(M+1): 322

### 6-Methyl-2-thioxo-1,2,3,4-tetrahydro-pyrimidine-5-carboxylic acid 3-methoxy-benzyl ester (120)

¹H NMR(CDCl₃): δ 7.47(s, 1H), 7.30-6.86(m, 4H), 6.79(s, 1H), 5.14(s, 2H), 4.18(s, 3H), 3.81(s, 3H), 2.30(s, 3H)

MS(M+1): 322

### 6-Methyl-2-thioxo-1,2,3,4-tetrahydro-pyrimidine-5-carbothioic acid S-(4-methoxy-benzyl) ester (121)

¹H NMR(CDCl₃): δ 7.40(s, 1H), 7.24(d, 2H), 6.85(d, 2H), 6.83(s, 1H), 4.25(s, 2H), 4.15(s, 2H), 3.79(s, 3H), 2.31(s, 3H)

MS(M+1): 309

### 6-Methyl-2-thioxo-1,2,3,4-tetrahydro-pyrimidine-5-carboxylic acid 2,4-dimethyl-benzyl ester (122)

¹H NMR(CDCl₃): δ 7.41(s, 1H), 7.19-6.99(m, 3H), 6.72(s, 1H), 5.15(s, 2H), 4.14(s, 2H), 2.32(d, 6H), 2.28(s, 3H)

MS(M+1): 291

### 6-Methyl-2-thioxo-1,2,3,4-tetrahydro-pyrimidine-5-carboxylic acid indan-1-yl ester (123)

¹H NMR(DMSO-d₆): δ 9.97(s, 1H), 8.94(s, 1H), 7.37-7.20(m, 4H), 6.13-6.10(m, 1H), 3.85(s, 2H), 3.00-2.97(m, 1H), 2.88-2.85(m, 1H), 2.50-2.44(m, 1H), 2.18(s, 3H), 2.00-1.96(m, 1H)

MS(M+1): 289

### 6-Methyl-2-thioxo-1,2,3,4-tetrahydro-pyrimidine-5-carboxylic acid indan-2-yl ester (124)

¹H NMR(DMSO-d₆): δ 9.94(s, 1H), 8.91(s, 1H), 7.25-7.15(m, 4H), 5.47-5.44(m, 1H), 3.78(s, 2H), 3.32-3.25(m, 2H), 2.51-2.50(m, 2H), 2.12(s, 3H)

MS(M+1): 289

### 6-Methyl-2-thioxo-1,2,3,4-tetrahydro-pyrimidine-5-carboxylic acid 2-(indan-1-yloxy)-ethyl ester (125)

¹H NMR(DMSO-d₆): δ 9.96(s, 1H), 8.97(s, 1H), 7.33-7.19(m, 4H), 4.91-4.90(m, 1H), 4.16(s, 2H), 3.87(s, 2H), 3.69(s, 2H), 2.98-2.90(m, 1H), 2.78-2.71(m, 1H), 2.31-2.24(m, 1H), 2.16(s, 3H), 1.94-1.89(m, 1H)

MS(M+1): 333

### 6-Methyl-2-thioxo-1,2,3,4-tetrahydro-pyrimidine-5-carboxylic acid 2-indan-1-yl-ethyl ester (126)

¹H NMR(DMSO-d₆): δ 9.96(s, 1H), 8.95(s, 1H), 7.19-7.12(m, 4H), 4.15(s, 2H), 3.90(s, 2H), 2.86-2.75(m, 2H), 2.25-2.14(m, 4H), 1.66-1.65(m, 2H)

MS(M+1): 317

### 6-Methyl-2-thioxo-1,2,3,4-tetrahydro-pyrimidine-5-carboxylic acid 1,1-dioxo-1H-116-benzo[b]thiophen-2-ylmethyl ester (127)

¹H NMR(DMSO-d₆): δ 10.08 (s, 1H), 9.07 (s, 1H), 7.87 (d, 1H), 7.75-7.55 (m, 4H), 5.13 (s, 2H), 3.93 (s, 2H), 2.21 (s, 3H)

MS(M+1): 351

### 6-Methyl-2-thioxo-1,2,3,4-tetrahydro-pyrimidine-5-carboxylic acid 5-(4-nitro-phenyl)-furan-2-ylmethyl ester (128)

¹H NMR(DMSO-d₆): δ 10.05 (s, 1H), 8.99 (s, 1H), 8.29 (d, 2H), 7.95 (d, 2H), 7.30 (d, 2H), 6.74 (d, 2H), 5.18 (s, 2H), 3.89 (s, 2H), 2.20 (s, 3H)

MS(M+1): 374

### 6-Methyl-2-thioxo-1,2,3,4-tetrahydro-pyrimidine-5-carboxylic acid but-3-inyl ester (129)

¹H NMR(DMSO-d₆): δ 10.00 (s, 1H), 9.01 (s, 1H), 4.09 (t, 2H), 3.89 (s, 2H), 2.87 (t, 1H), 2.19 (s, 3H)

MS(M+1): 225

### 6-Methyl-2-thioxo-1,2,3,4-tetrahydro-pyrimidine-5-carboxylic acid pent-4-inyl ester (130)

¹H NMR(DMSO-d₆): δ 9.97 (s, 1H), 8.97 (s, 1H), 4.08 (t, 2H), 3.89 (s, 2H), 2.81 (t, 1H), 2.27-2.22 (m, 2H), 2.17 (s, 3H), 1.80-1.72 (m, 2H)

MS(M+1): 239

### 6-Methyl-2-thioxo-1,2,3,4-tetrahydro-pyrimidine-5-carboxylic acid 3,5-dichloro-benzyl ester (131)

¹H NMR(CDCl₃): δ 7.33(m, 2H), 7.22(m, 2H), 6.70(s, 1H), 5.11(s, 2H), 4.19(s, 2H), 2.30(s, 3H)

MS(M+1): 332

### 6-Methyl-2-thioxo-1,2,3,4-tetrahydro-pyrimidine-5-carboxylic acid 5-chloro-2-methoxy-benzyl ester (132)

¹H NMR(CDCl₃): δ 7.25(m, 2H), 7.19(s, 1H), 6.83(d, 1H), 6.54(s, 1H), 4.19(s, 2H), 2.30(s, 3H)

MS(M+1): 327

### 6-Methyl-2-thioxo-1,2,3,4-tetrahydro-pyrimidine-5-carboxylic acid 2-o-tolyl-ethyl ester (133)

¹H NMR(CDCl₃): δ 7.49(s, 1H), 7.19-7.14(m, 4H), 6.83(s, 1H), 4.35(t, 2H), 4.10(s, 2H), 2.97(t, 3H), 2.34(s, 3H), 2.25(s, 3H)

MS(M+1): 291

### 6-Methyl-2-thioxo-1,2,3,4-tetrahydro-pyrimidine-5-carboxylic acid 3,5-difluoro-benzyl ester (134)

¹H NMR(CDCl₃): δ 6.86(m, 2H), 6.77(m, 1H), 6.61(s, 1H), 5.14(s, 2H), 4.20(s, 2H), 2.31(s, 3H)

MS(M+1): 299

### 6-Methyl-2-thioxo-1,2,3,4-tetrahydro-pyrimidine-5-carboxylic acid 3-methyl-4-nitro-benzyl ester (135)

¹H NMR(CDCl₃): δ 8.01(m, 1H), 7.31-7.26(m, 2H), 6.56(s, 1H), 5.19(s, 2H), 4.20(s, 2H), 2.62(s, 3H), 2.30(s, 3H)

MS(M+1): 322

### 6-Methyl-2-thioxo-1,2,3,4-tetrahydro-pyrimidine-5-carboxylic acid 3-nitro-4-methyl-benzyl ester (136)

¹H NMR(CDCl₃): δ 7.96(s, 1H), 7.50-7.35(m, 3H), 6.73(s, 1H), 5.20(s, 2H), 4.18(s, 2H), 2.61(s, 3H), 2.30(s, 3H)

MS(M+1): 337

### 6-Methyl-2-thioxo-1,2,3,4-tetrahydro-pyrimidine-5-carboxylic acid 2,4-dimethoxy-3-methyl-benzyl ester (137)

¹H NMR(CDCl₃): δ 7.23(s, 1H), 7.15(d, 1H), 6.63(d, 1H), 6.58(s, 1H), 2H), 4.14(s, 2H), 3.84(s, 3H), 3.71(s, 3H), 2.28(s, 3H), 2.13(s, 3H)

MS(M+1): 337

### 6-Methyl-2-thioxo-1,2,3,4-tetrahydro-pyrimidine-5-carboxylic acid 4-methoxy-2,3-dimethyl-benzyl ester (138)

¹H NMR(CDCl₃): δ 7.31(s, 1H), 7.15(d, 1H), 6.69(d, 1H), 6.64(s, 1H), 5.15(s, 2H), 4.13(s, 2H), 3.83(s, 3H), 2.28(s, 3H), 2.22(s, 3H), 2.15(s, 3H)

MS(M+1): 321

### 6-Methyl-2-thioxo-1,2,3,4-tetrahydro-pyrimidine-5-carboxylic acid thiophen-3-ylmethyl ester (139)

¹H NMR(DMSO-d₆): δ 10.00(s, 1H), 8.98(s, 1H), 7.54-7.49(m, 2H), 7.11-7.10(m, 1H), 5.08(s, 2H), 3.89(s, 2H), 2.18(s, 3H)

MS(M+1): 269

### 6-Methyl-2-thioxo-1,2,3,4-tetrahydro-pyrimidine-5-carboxylic acid 6-nitro-benzo[1,3]dioxol-5-ylmethyl ester (140)

¹H NMR(DMSO-d₆): δ 10.04(s, 1H), 9.01(s, 1H), 7.69(s, 1H), 7.13(s, 1H), 6.24(s, 2H), 5.32(s, 2H), 3.92(s, 2H), 2.17(s, 3H)

MS(M+1): 352

### 6-Methyl-2-thioxo-1,2,3,4-tetrahydro-pyrimidine-5-carboxylic acid 3-bromo-4-methoxy-benzyl ester (141)

¹H NMR(DMSO-d₆): δ 10.00(s, 1H), 8.97(s, 1H), 7.90(d, 1H), 7.37-7.34(m, 1H), 7.10-7.08(m, 1H), 5.01(s, 2H), 3.88(s, 2H), 3.83(s, 3H), 2.17(s, 3H)

MS(M+1): 372

### 6-Methyl-2-thioxo-1,2,3,4-tetrahydro-pyrimidine-5-carboxylic acid 2,4,6-trimethyl-benzyl ester (142)

¹H NMR(DMSO-d₆): δ 9.96(s, 1H), 8.92(s, 1H), 6.84(s, 2H), 5.09(s, 2H), 3.81(s, 2H), 2.27(s, 6H), 2.20(s, 3H), 2.07(s, 3H)

MS(M+1): 305

### 6-Methyl-2-thioxo-1,2,3,4-tetrahydro-pyrimidine-5-carboxylic acid 5-chloro-2-nitro-benzyl ester (143)

¹H NMR(DMSO-d₆): δ 10.07(s, 1H), 9.03(s, 1H), 8.14-8.12(m, 1H), 7.71-7.69(m, 2H), 5.40(s, 2H), 3.92(s, 2H), 2.17(s, 3H)

MS(M+1): 342

### 2-Thioxo-6-trifluoromethyl-1,2,3,4-tetrahydro-pyrimidine-5-carboxylic acid benzyl ester (146)

¹H NMR(CDCl3): δ 7.81(s, 1H), 7.34-7.40(m, 5H), 7.26(s, 1H), 5.26(s, 2H), 3.88(s, 2H)

MS(M+1): 317

### 6-Methyl-2-thioxo-1,2,3,4-tetrahydro-pyrimidine-5-carboxylic acid 9-ethyl-9H-carbazol-3-ylmethyl ester (147)

¹H NMR(DMSO-d₆): δ 9.97(s, 1H), 8.96(s, 1H), 8.176(m, 2H), 7.61-7.48(m, 4H), 7.21(m, 1H), 5.27(s, 2H), 4.45(q, 2H), 3.92(s, 2H), 2.21(s, 3H), 1.30(t, 3H)

MS(M+1): 380

### 6-Methyl-2-thioxo-1,2,3,4-tetrahydro-pyrimidine-5-carboxylic acid 3,5-bis-trifluoromethyl-benzyl ester (148)

¹H NMR(CDCl3): δ 7.86(s, 1H), 7.80(s, 2H), 7.51(s, 1H), 6.81(s, 1H), 5.28(s, 2H), 4.20(s, 2H), 2.32(s, 3H)

MS(M+1): 399

### 6-Methyl-2-thioxo-1,2,3,4-tetrahydro-pyrimidine-5-carboxylic acid 2,5-dimethyl-benzyl ester (149)

¹H NMR(CDCl3): δ 7.30(s, 1H), 7.11-7.08(m, 3H), 6.62(s, 1H), 5.15(s, 2H), 4.1(s, 2H), 2.32(s, 3H), 2.30(s, 3H)

MS(M+1): 291

### 6-Methyl-2-thioxo-1,2,3,4-tetrahydro-pyrimidine-5-carboxylic acid 3,5-diethoxy-benzyl ester (150)

¹H NMR(CDCl3): δ 7.34(s, 1H), 6.67(s, 1H), 6.45-6.40(m, 3H), 5.09(s, 2H), 4.18(s, 2H), 4.02(q, 4H), 2.30(s, 3H), 1.41(t, 6H)

MS(M+1): 351

### 6-Methyl-2-thioxo-1,2,3,4-tetrahydro-pyrimidine-5-carboxylic acid bicyclo[2,2,1]hept-2-ylmethyl ester (151)

¹H NMR(CDCl3): δ 7.36(s, 1H), 6.71(s, 1H), 4.15(s, 2H), 4.02(d, 2H), 2.29(s, 3H), 2.19(m, 2H), 1.73(m, 1H), 1.47-1.10(m, 8H)

MS(M+1): 281

### 6-Methyl-2-thioxo-1,2,3,4-tetrahydro-pyrimidine-5-carboxylic acid 4-ethyl-benzyl ester (152)

¹H NMR(CDCl3): δ 7.44(s, 1H), 7.26(d, 2H), 7.21(d, 2H), 6.76(s, 1H), 5.14(s, 2H), 4.16(s, 2H), 2.67(q, 2H), 2.30(s, 3H), 1.24(t, 3H)

MS(M+1): 291

### 6-Methyl-2-thioxo-1,2,3,4-tetrahydro-pyrimidine-5-carboxylic acid thiophen-2-ylmethyl ester (153)

¹H NMR(CDCl3): δ 7.33(d, 1H), 7.29(s, 1H), 7.09(d, 1H), 6.99(m, 1H), 6.76(s, 1H), 5.33(s, 2H), 4.15(s, 2H), 2.30(s, 3H)

MS(M+1): 269

### 6-Methyl-2-thioxo-1,2,3,4-tetrahydro-pyrimidine-5-carboxylic acid pyridin-2-ylmethyl ester (154)

¹H NMR(CDCl3): δ 8.61(d, 2H), 7.71(m, 1H), 7.31(m, 2H), 6.66(m, 1H), 5.29(s, 2H), 4.23(s, 2H), 2.31(s, 3H)

MS(M+1): 264

### 6-Methyl-2-thioxo-1,2,3,4-tetrahydro-pyrimidine-5-carboxylic acid pyridin-4-ylmethyl ester (155)

¹H NMR(CDCl3): δ 8.63(d, 2H), 7.24(d, 2H), 6.62(m, 1H), 5.19(s, 2H), 4.23(s, 2H), 2.31(s, 3H)

MS(M+1): 264

### 6-Methyl-2-thioxo-1,2,3,4-tetrahydro-pyrimidine-5-carboxylic acid 2,3-dimethyl-benzyl ester (156)

¹H NMR(CDCl3): δ 7.28(s, 1H), 7.17-7.10(m, 3H), 6.59(s, 1H), 5.20(s, 2H), 4.16(t, 2H), 2.31(d, 6H), 2.24(s, 3H)

MS(M+1): 291

### 6-Methyl-2-thioxo-1,2,3,4-tetrahydro-pyrimidine-5-carboxylic acid hex-3-inyl ester (157)

¹H NMR(DMSO-d₆): δ 9.99 (s, 1H), 9.00 (s, 1H), 4.06 (t, 2H), 3.89 (s, 2H), 2.50-2.45 (m, 2H), 2.19 (s, 3H), 2.15-2.10 (m, 2H), 1.03 (t, 3H)

MS(M+1): 253

### 6-Methyl-2-thioxo-1,2,3,4-tetrahydro-pyrimidine-5-carboxylic acid 3-pyridin-3-yl-propyl ester (158)

¹H NMR(DMSO-d₆): δ 9.98 (s, 1H), 8.99 (s, 1H), 8.67 (s, 1H), 8.64 (d, 1H), 8.15 (bd, 1H), 7.72 (bt, 1H), 4.06 (t, 2H), 3.86 (s, 2H), 2.78 (t, 2H), 2.18 (s, 3H), 2.22-2.95 (m, 2H)

MS(M+1): 292

### 6-Methyl-2-thioxo-1,2,3,4-tetrahydro-pyrimidine-5-carboxylic acid prop-2-inyl ester (159)

¹H NMR(DMSO-d₆): δ 10.06 (s, 1H), 9.02 (s, 1H), 4.70 (s, 2H), 3.89 (s, 2H), 3.52 (t, 1H), 2.19 (s, 3H)

MS(M+1): 211

### 6-Methyl-2-thioxo-1,2,3,4-tetrahydro-pyrimidine-5-carboxylic acid pent-3-inyl ester (160)

¹H NMR(DMSO-d₆): δ 9.99 (s, 1H), 8.99 (s, 1H), 4.05 (t, 2H), 3.88 (s, 2H), 2.50-2.45 (m, 2H), 2.19 (s, 3H), 1.74 (s, 3H)

MS(M+1): 239

### 6-Methyl-2-thioxo-1,2,3,4-tetrahydro-pyrimidine-5-carboxylic acid tetrahydro-furan-2-ylmethyl ester (161)

¹H NMR(DMSO-d₆): δ 9.98 (s, 1H), 8.97 (s, 1H), 4.04-3.96 (m, 3H), 3.88 (s, 2H), 3.75-3.61 (m, 2H), 2.18 (s, 3H), 1.92-1.78 (m, 3H), 1.60-1.50 (m, 1H)

MS(M+1): 257

### 6-Methyl-2-thioxo-1,2,3,4-tetrahydro-pyrimidine-5-carboxylic acid tetrahydrofuran-3-ylmethyl ester (162)

¹H NMR(DMSO-d₆): δ 9.97 (s, 1H), 8.97 (s, 1H), 4.04-3.92 (m, 2H), 3.89 (s, 2H), 3.75-3,58 (m, 3H), 3.45-3.40 (m, 1H), 2.17 (s, 3H), 1.95-1.90 (m, 1H), 1.60-1.52 (m, 1H)

MS(M+1): 257

### 6-Methyl-2-thioxo-1,2,3,4-tetrahydro-pyrimidine-5-carboxylic acid 2-phenoxy-ethyl ester (163)

¹H NMR(DMSO-d₆): δ 10.00 (s, 1H), 8.98(s, 1H), 7.28 (t, 2H), 6.95 (d, 3H), 4.35-4.33 (m, 2H), 4.20-4.18 (m, 2H), 3.87 (s, 2H), 2.17 (s, 3H)

MS(M+1): 293

### 6-Methyl-2-thioxo-1,2,3,4-tetrahydro-pyrimidine-5-carboxylic acid 3-trifluoromethylsulfinyl-benzyl ester (164)

¹H NMR(DMSO-d₆): δ 10.03 (s, 1H), 9.01 (s, 1H), 7.72-7.55 (m, 4H), 5.17 (s, 2H), 3.93 (s, 2H), 2.19 (s, 3H)

MS(M+1): 363

### 6-Methyl-2-thioxo-1,2,3,4-tetrahydro-pyrimidine-5-carboxylic acid

### 2-(1-methyl-1H-indol-3-yl)-ethyl ester (165)

¹H NMR(DMSO-d₆): δ 10.00 (s, 1H), 9.01 (s, 1H), 7.59 (d, 1H), 7.43 (d, 1H), 7.19 (s, 1H), 7.18 (t, 1H), 7.06 (t, 1H), 4.30 (t, 2H), 3.91 (s, 2H), 3.77 (s, 3H), 3.04 (t, 2H), 2.18 (s, 3H)

MS(M+1): 330

### 6-(3-Ethoxycarbonyl-propyl)-2-thioxo-1,2,3,4-tetrahydro-pyrimidine-5-carboxylic acid benzyl ester (169)

¹H NMR(CDCl3): δ 8.06(s, 1H), 7.32-7.39(m, 5H), 7.02(s, 1H), 5.16(s, 2H), 4.16(s, 2H), 4.13(q, 2H), 2.72(t, 2H), 2.33(t, 2H), 1.84-1.91(m, 2H), 1.25(t, 3H),

MS(M+1): 363

### 6-(3-Carboxy-propyl)-2-thioxo-1,2,3,4-tetrahydro-pyrimidine-5-carboxylic acid benzyl ester (170)

¹H NMR(DMSO-d₆): δ 12.00(bs, 1H), 10.00(s, 1H), 8.95(b, 1H), 7.36(bs, 5H), 5.11(s, 2H), 3.92(s, 2H), 2.62(t, 2H), 2.15(t, 2H), 1.65-1.75(m, 2H)

MS(M+1): 335

### 6-Methyl-2-thioxo-1,2,3,4-tetrahydro-pyrimidine-5-carboxylic acid 2-m-tolyl-ethyl ester (172)

¹H NMR(CDCl3): δ 7.59(s, 1H), 7.22-6.99(m, 4H), 6.93(s, 1H), 4.36(t, 2H), 4.09(s, 2H), 2.94(t, 2H), 2.34(s, 3H), 2.23(s, 3H)

MS(M+1): 291

### 6-Methyl-2-thioxo-1,2,3,4-tetrahydro-pyrimidine-5-carboxylic acid 2-p-tolyl-ethyl ester (173)

¹H NMR(CDCl3): δ 7.33(s, 1H), 7.13-7.07(m, 4H), 6.69(s, 1H), 4.35(t, 2H), 4.09(s, 2H), 2.94(t, 2H), 2.34(s, 3H), 2.23(s, 3H)

MS(M+1): 291

### 6-Methyl-2-thioxo-1,2,3,4-tetrahydro-pyrimidine-5-carboxylic acid 2,5-dimethoxy-benzyl ester (174)

¹H NMR(CDCl3): δ 7.44(s, 1H), 6.86-6.82(m, 3H), 6.77(s, 1H), 5.19(s, 2H), 4.17(s, 2H), 3.79(d, 6H), 2.30(s, 3H)

MS(M+1): 323

### 6-Methyl-2-thioxo-1,2,3,4-tetrahydro-pyrimidine-5-carboxylic acid 2-(3,5-dimethyl-phenyl)-ethyl ester (175)

¹H NMR(CDCl3): δ 7.28(s, 1H), 6.88-6.82(m, H), 6.64(s, 1H), 4.35(t, 2H), 4.10(s, 2H), 2.89(t, 2H), 2.30(s, 6H), 2.23(s, 3H)

MS(M+1): 305

### 6-Methyl-2-thioxo-1,2,3,4-tetrahydro-pyrimidine-5-carboxylic acid 4-methoxy-3-methyl-benzyl ester (176)

¹H NMR(CDCl3): δ 7.40(s, 1H), 7.17-7.12(m, 2H), 6.81(d, 1H), 6.74(s, 1H), 5.08(s, 2H), 4.15(s, 2H), 3.84(s, 3H), 2.29(s, 3H), 2.30(s, 3H)

MS(M+1): 307

### 6-Methyl-2-thioxo-1,2,3,4-tetrahydro-pyrimidine-5-carboxylic acid 2-fluoro-4-methoxy-benzyl ester (177)

¹H NMR(CDCl3): δ 7.30(s, 1H), 7.25(s, 1H), 6.69-6.62(m, 2H), 6.59(s, 1H), 5.16(s, 2H), 4.14(s, 2H), 3.81(s, 3H), 2.27(s, 3H)

MS(M+1): 311

### 6-Methyl-2-thioxo-1,2,3,4-tetrahydro-pyrimidine-5-carboxylic acid 3-bromo-4,5-dimethoxy-benzyl ester (178)

¹H NMR(CDCl3): δ 7.38(s, 1H), 7.13(s, 1H), 6.83(s, 1H), 6.72(s, 1H), 5.08(s, 2H), 4.17(s, 2H), 3.87(d, 6H), 2.30(s, 3H)

MS(M+1): 402

### 6-Methyl-2-thioxo-1,2,3,4-tetrahydro-pyrimidine-5-carboxylic acid 4-methoxy-3,5-dimethyl-benzyl ester (180)

¹H NMR(DMSO-d₆): δ 9.98(s, 1H), 8.94(s, 1H), 7.01(s, 2H), 4.98(s, 2H), 3.89(s, 2H), 3.31(s, 3H), 2.21(s, 6H), 2.19(s, 3H)

MS(M+1): 321

### 6-Methyl-2-thioxo-1,2,3,4-tetrahydro-pyrimidine-5-carboxylic acid 3,5-dimethyl-1-phenyl-1H-pyrazol-4-ylmethyl ester (181)

¹H NMR(DMSO-d₆): δ 9.96(s, 1H), 8.92(s, 1H), 7.51-7.31(m, 5H), 5.00(s, 2H), 3.85(s, 2H), 2.29(s, 3H), 2.20(s, 3H), 2.18(s, 3H)

MS(M+1): 357

### 6-Methyl-2-thioxo-1,2,3,4-tetrahydro-pyrimidine-5-carboxylic acid 2-nitro-benzyl ester (183)

¹H NMR(CDCl3): δ 8.09(d, 1H), 7.67-7.05(m, 3H), 7.23(s, 1H), 6.56(s, 1H), 5.56(s, 2H), 4.19(s, 2H), 2.30(s, 3H)

MS(M+1): 308

### 6-Methyl-2-thioxo-1,2,3,4-tetrahydro-pyrimidine-5-carboxylic acid 2,6-dimethyl-benzyl ester (184)

¹H NMR(CDCl3): δ 7.20(s, 1H), 7.18-7.05(m, 3H), 6.51(s, 1H), 5.26(s, 2H), 4.11(s, 2H), 2.38(s, 6H), 2.27(s, 3H)

MS(M+1): 291

### 6-Methyl-2-thioxo-1,2,3,4-tetrahydro-pyrimidine-5-carboxylic acid 5-bromo-2-methoxy-benzyl ester (185)

¹H NMR(CDCl3): δ 77.43-7.37(m, 2H), 7.16(s, 1H), 6.76(d, 1H), 6.51(s, 1H), 5.17(s, 2H), 4.19(s, 2H), 3.83(s, 3H), 2.29(s, 3H)

MS(M+1): 372

### 6-Methyl-2-thioxo-1,2,3,4-tetrahydro-pyrimidine-5-carboxylic acid 3-phenyl-prop-2-inyl ester (192)

¹H NMR(DMSO-d₆): δ 10.05 (s, 1H), 9.01 (s, 1H), 7.50-7.35 (m, 5H), 4.95 (s, 2H), 3.91 (s, 2H), 2.20 (s, 3H)

MS(M+1): 287

### 6-Methyl-2-thioxo-1,2,3,4-tetrahydro-pyrimidine-5-carboxylic acid 2,3-dibromo-allyl ester (193)

¹H NMR(DMSO-d₆): δ 10.07 (s, 1H), 9.03 (s, 1H), 7.19 (s, 1H), 4.92 (s, 2H), 3.91 (s, 2H), 2.19 (s, 3H)

MS(M+1): 371

### 6-Methyl-2-thioxo-1,2,3,4-tetrahydro-pyrimidine-5-carboxylic acid 2,3-dihvdrobenzo[1,4]dioxin-2-ylmethyl ester (195)

¹H NMR(DMSO-d): δ 10.00 (s, 1H), 8.97 (s, 1H), 6.90-6.80 (m, 4H), 4.46-4.42 (m, 1H), 4.35-4.27 (m, 3H), 4.01 (q, 1H), 3.88 (s, 3H), 2.17 (s, 3H)

MS(M+1): 321

### 2-Thioxo-1,2,3,6-tetrahydro-pyrimidine-4,5-dicarboxylic acid 5-benzyl ester 4-methyl ester (196)

¹H NMR(DMSO-d₆): δ 10.72(s, 2H), 9.13(s, 1H), 7.35(bs, 5H), 5.09(s, 2H), 4.01(s, 2H), 3.76(s, 3H)

MS(M+1): 307

### 6-Methyl-2-thioxo-1,2,3,4-tetrahydro-pyrimidine-5-carboxylic acid 5-ethoxycarbonyl-pentyl ester (197)

¹H NMR(DMSO-d₆): δ 9.93 (s, 1H), 8.95 (s, 1H), 4.05-4.00 (m, 4H), 3.86 (s, 3H), 2.26 (t, 2H), 2.16 (s, 3H), 1.60-1.50 (m, 4H), 1.35-1.17 (m, 2H), 1.15 (t, 3H)

MS(M+1): 315

### 6-Methyl-2-thioxo-1,2,3,4-tetrahydro-pyrimidine-5-carboxylic acid 9-methoxycarbonyl-nonyl ester (198)

¹H NMR(DMSO-d₆): δ 9.93 (s, 1H), 8.93 (s, 1H), 4.05-3.97 (m, 2H), 3.86 (s, 3H), 3.56 (s, 3H), 2.26 (t, 2H), 2.16 (s, 3H), 1.60-1.40 (m, 4H), 1.23 (bs, 10H)

MS(M+1): 357

### 6-Methyl-2-thioxo-1,2,3,4-tetrahydro-pyrimidine-5-carboxylic acid 4-trifluoromethylsulfinyl-benzyl ester (199)

¹H NMR(DMSO-d₆): δ 10.03 (s, 1H), 9.00 (s, 1H), 7.72 (d, 2H), 7.50 (d, 2H), 5.17 (s, 2H), 3.93 (s, 2H), 2.19 (s, 3H)

MS(M+1): 363

### 6-Methyl-2-thioxo-1,2,3,4-tetrahydro-pyrimidine-5-carboxylic acid dec-3-inyl ester (200)

¹H NMR(DMSO-d₆): δ 9.97 (s, 1H), 8.99 (s, 1H), 4.05 (t, 2H), 3.87 (s, 2H), 2.50-2.45 (m, 2H), 2.17 (s, 3H), 2.12-2.07 (m, 2H), 1.40-1.10 (m, 8H), 0.84.(t, 3H)

MS(M+1): 309

### 6-Methyl-2-thioxo-1,2,3,4-tetrahydro-pyrimidine-5-carboxylic acid 2-(toluene-4-sulfonyl)-ethyl ester (201)

¹H NMR(DMSO-d₆): δ 9.93 (s, 1H), 8.95 (s, 1H), 7.73 (d, 2H), 7.43 (d, 2H), 4.28 (t, 2H), 3.69 (t, 2H), 3.51 (s, 2H), 2.39 (s, 3H), 2.03 (s, 3H)

MS(M+1): 355

### 6-Methyl-2-thioxo-1,2,3,4-tetrahydro-pyrimidine-5-carboxylic acid 3-methyl-thiophen-2-ylmethyl ester (202)

¹H NMR(CDCl3): δ 6.23(d, 1H), 6.89(d, 1H), 6.59(s, 1H), 5.27(s, 2H), 4.15(s, 2H), 2.29(s, 3H), 2.27(s, 3H)

MS(M+1): 283

### 6-Methyl-2-thioxo-1,2,3,4-tetrahydro-pyrimidine-5-carboxylic acid 5-bromo-thiophen-2-ylmethyl ester (203)

¹H NMR(CDCl3): δ 7.30(s, 1H), 6.93(d, 1H), 6.84(d, 1H), 6.63(s, 1H), 5.23(s, 2H), 4.15(s, 2H), 2.29(s, 3H)

MS(M+1): 348

### 6-Methyl-2-thioxo-1,2,3,4-tetrahydro-pyrimidine-5-carboxylic acid 4-bromo-thiophen-2-ylmethyl ester (204)

¹H NMR(CDCl3): δ 7.23(s, 1H), 7.18(s, 1H), 7.01(s, 1H), 6.53(s, 1H), 5.27(s, 2H), 4.16(s, 2H), 2.30(s, 3H)

MS(M+1): 348

### 6-Methyl-2-thioxo-1,2,3,4-tetrahydro-pyrimidine-5-carboxylic acid 5-ethyl-thiophen-2-ylmethyl ester (205)

¹H NMR(CDCl3): δ 7.42(s, 1H), 6.89(d, 1H), 6.74(s, 1H), 6.65(d, 1H), 5.25(s, 2H), 4.15(s, 2H), 2.82(q, 2H), 2.30(s, 3H), 1.33(t, 2H)

MS(M+1): 297

### 6-Methyl-2-thioxo-1,2,3,4-tetrahydro-pyrimidine-5-carboxylic acid 2-thiophen-2-yl-ethyl ester (206)

¹H NMR(CDCl3): δ 7.18(d, 1H), 6.95(t, 1H), 6.85(d, 1H), 6.63(s, 1H), 4.39(t, 2H), 4.15(s, 2H), 3.18(t, 2H), 2.25(s, 3H)

MS(M+1): 283

### 6-Methyl-2-thioxo-1,2,3,4-tetrahydro-pyrimidine-5-carboxylic acid

### 3-chloro-4-methyl-thiophen-2-ylmethyl ester (207)

¹H NMR(CDCl3): δ 7.00(s, 1H), 6.59(s, 1H), 5.28(s, 2H), 4.16(s, 2H), 2.30)s, 3H), 2.21(s, 3H)

MS(M+1): 317

### 6-Methyl-2-thioxo-1,2,3,4-tetrahydro-pyrimidine-5-carboxylic acid 2-thiophen-3-yl-ethyl ester (208)

¹H NMR(CDCl3): δ 7.52(s, 1H), 7.29(d, 1H), 7.01(s, 1H), 6.96(d, 1H), 6.86(s, 1H), 4.38(t, 2H), 4.13(s, 2H), 3.01(t, 2H), 2.23(s, 3H)

MS(M+1): 283

### 6-Methyl-2-thioxo-1,2,3,4-tetrahydro-pyrimidine-5-carboxylic acid 2-4-[(2-methoxyethyl)-methyl-amino]-phenyl-ethyl ester (209)

¹H NMR(DMSO-d₆): δ 9.95(s, 1H), 8.96(s, 1H), 7.01(d, 2H), 6.62(d, 2H), 4.15(t, 2H), 3.85(s, 2H), 3.45(s, 3H), 3.24(s, 3H), 2.87(s, 3H), 2.75(t, 2H), 2.13(s, 3H)

MS(M+1): 364

### 6-Methyl-2-thioxo-1,2,3,4-tetrahydro-pyrimidine-5-carboxylic acid 2-thiophen-2-yl-thiazol-5-ylmethyl ester (210)

¹H NMR(CDCl3): δ 7.52(bs, 1H), 7.50(d, 1H), 7.40(dd, 1H), 7.08(dd, 1H), 6.92(bs, 1H), 6.89(s, 1H), 5.01(s, 2H), 4.11(s, 2H), 2.27(s, 3H)

MS(M+1): 352

### 4-Fluoromethyl-6-methyl-2-thioxo-1,2,3,4-tetrahydro-pyrimidine-5-carboxylic acid benzyl ester (211)

¹H NMR(DMSO-d₆): δ 10.35 (s, 1H), 9.40 (s, 1H), 7.40-7.31 (m, 5H), 5.14 (q, 2H), 4.38-4.24 (m, 2H), 4.23-4.15 (m, 1H), 2.25 (s, 3H)

MS(M+1): 295

### 6-Methyl-2-thioxo-1,2,3,4-tetrahydro-pyrimidine-5-carboxylic acid 3-methyl-thieno[2,3-clisothiazol-5-ylmethyl ester (213)

¹H NMR(DMSO-d₆): δ 9.64 (s, 1H), 8.67 (s, 1H), 6.89 (s, 1H), 5.25 (s, 2H), 4.05 (s, 2H), 2.73 (s, 3H), 2.28 (s, 3H)

MS(M+1): 340

### 6-Methyl-2-thioxo-1,2,3,4-tetrahydro-pyrimidine-5-carboxylic acid 4-benzyloxy-but-2-inyl ester (214)

¹H NMR(DMSO-d₆): δ 10.05 (s, 1H), 9.01 (s, 1H), 7.38-7.25 (m, 5H), 4.84 (s, 2H), 4.49 (s, 2H), 4.22 (s, 2H), 4.89 (s, 2H), 2.18 (s, 3H)

MS(M+1): 331

### 6-Methyl-2-thioxo-1,2,3,4-tetrahydro-pyrimidine-5-carboxylic acid 4-methoxy-but-2-inyl ester (215)

¹H NMR(DMSO-d₆): δ 10.05 (s, 1H), 9.00 (s, 1H), 4.76 (s, 2H), 4.11 (s, 2H), 3.88 (s, 2H), 3.23 (s, 3H), 2.17 (s, 3H)

MS(M+1): 255

### 6-Methyl-2-thioxo-1,2,3,4-tetrahydro-pyrimidine-5-carboxylic acid 2,6-dimethyl-thieno[3,2-d]thiazol-5-ylmethyl ester (216)

¹H NMR(DMSO-d₆): δ 10.04 (s, 1H), 9.00 (s, 1H), 5.30 (s, 2H), 3.88 (s, 2H), 2.75 (s, 3H), 2.38 (s, 3H), 2.19 (s, 3H)

MS(M+1): 354

### 6-Methyl-2-thioxo-1,2,3,4-tetrahydro-pyrimidine-5-carboxylic acid 5-methylsulfanyl-thiophen-2-ylmethyl ester (217)

¹H NMR(CDCl3): δ 7.31(s, 1H), 6.94-6.91(m, 2H), 6.29(s, 1H), 5.24(s, 2H), 4.16(s, 2H), 2.50(s, 3H), 2.30(s, 3H)

MS(M+1): 315

### 6-Methyl-2-thioxo-1,2,3,4-tetrahydro-pyrimidine-5-carboxylic acid [2,240 ]bithiophenyl-5-ylmethyl ester (218)

¹H NMR(CDCl3): δ 7.29(s, 1H), 7.24-6.98(m, 5H), 6.62(s, 1H), 5.28(s, 2H), 4.17(s, 2H), 2.30(s, 3H)

MS(M+1): 351

### 6-Methyl-2-thioxo-1,2,3,4-tetrahydro-pyrimidine-5-carboxylic acid benzo[b]thiophen-2-ylmethyl ester (219)

¹H NMR(CDCl3): δ 7.83-7.75(m, 2H), 7.37-7.30(m, 4H), 6.63(s, 1H), 5.42(s, 2H), 4.19(s, 2H), 2.31 (s, 3H)

MS(M+1): 319

### 6-Methyl-2-thioxo-1,2,3,4-tetrahydro-pyrimidine-5-carboxylic acid 3-pyrrol-1-yl-thiophen-2-ylmethyl ester (220)

¹H NMR(CDCl3): δ 7.36(d, 1H), 7.20(s, 1H), 6.87(t, 1H), 6.53(s, 1H), 6.33(t, 1H), 5.25(s, 2H), 4.14(s, 2H), 2.30(s, 3H)

MS(M+1): 334

### 6-Methyl-2-thioxo-1,2,3,4-tetrahydro-pyrimidine-5-carboxylic acid 5-(2-methyl-thiazol-5-yl)-thiophen-2-ylmethyl ester (221)

¹H NMR(CDCl3): δ 7.28(d, 1H), 7.20(s, 1H), 7.02(d, 1H), 6.59(s, 1H), 5.32(s, 2H), 4.17(s, 2H), 2.75(s, 3H), 2.30(s, 3H)

MS(M+1): 366

### 6-Methyl-2-thioxo-1,2,3,4-tetrahydro-pyrimidine-5-carboxylic acid 5-(1-methyl-5-trifluoromethyl-1H-pyrazol-3-yl)-thiophen-2-ylmethyl ester (222)

¹H NMR(CDCl3): δ 7.36(s, 1H), 7.13-7.08(m, 2H), 6.69(s, 1H), 6.63(s, 1H), 5.34(s, 2H), 4.17(s, 2H), 4.02(s, 3H), 2.31(s, 3H)

MS(M+1): 417

### 6-Methyl-2-thioxo-1,2,3,4-tetrahydro-pyrimidine-5-carboxylic acid 1,3-dimethyl-1H-thieno[2,3-c]pyrazol-5-ylmethyl ester (223)

¹H NMR(CDCl3): δ 7.37(s, 1H), 6.93(s, 1H), 6.70(s, 1H), 5.26(s, 2H), 4.15(s, 2H), 3.88(s, 3H), 2.42(s, 3H), 2.30(s, 3H)

MS(M+1): 337

### 6-Methyl-2-thioxo-1,2,3,4-tetrahydro-pyrimidine-5-carboxylic acid 2-(benzothiazol-2-ylsulfanyl)-ethyl ester (224)

¹H NMR(DMSO-d₆): δ 9.95 (s, 1H), 8.94 (s, 1H), 8.00 (d, 1H), 7.84 (d, 1H), 7.45 (t, 1H), 7.35 (t, 1H), 4.40 (t, 2H), 3.82 (s, 2H), 3.65 (t, 2H), 2.14 (s, 3H)

MS(M+1): 366

### 6-Methyl-2-thioxo-1,2,3,4-tetrahydro-pyrimidine-5-carboxylic acid benzotriazol-1-ylmethyl ester (225)

¹H NMR(DMSO-d₆): δ 10.10 (s, 1H), 9.00 (s, 1H), 8.09 (d, 1H), 7.96 (d, 1H), 7.63 (t, 1H), 7.45 (t, 1H), 6.69 (s, 2H), 3.81 (s, 2H), 2.14 (s, 3H)

MS(M+1): 304

### 6-Methyl-2-thioxo-1,2,3,4-tetrahydro-pyrimidine-5-carboxylic acid benzothiazol-2-ylmethyl ester (226)

¹H NMR(DMSO-d₆): δ 10.13 (s, 1H), 9.09 (s, 1H), 8.12 (d, 1H), 8.00 (d, 1H), 7.52 (t, 1H), 7.45 (t, 1H), 5.52 (s, 2H), 3.99 (s, 2H), 2.23 (s, 3H)

MS(M+1): 320

### 6-Methyl-2-thioxo-1,2,3,4-tetrahydro-pyrimidine-5-carboxylic aid 4-(4-methoxy-benzyloxy)-benzyl ester (227)

¹H NMR(CDCl3): δ 7.37-7.29(m, 4H), 6.97-6.91(m, 4H), 6.60(s, 1H), 5.11(s, 2H), 4.99(s, 2H), 4.15(s, 2H), 3.83(s, 3H), 2.28(s, 3H)

MS(M+1): 399

### 6-Methyl-2-thioxo-1,2,3,4-tetrahydro-pyrimidine-5-carboxylic acid 4-t-butoxycarbonyloxy-benzyl ester (228)

¹H NMR(CDCl3): δ 7.37(d, 2H), 7.21(s, 1H), 7.17(d, 2H), 6.55(s, 1H), 5.15(s, 2H), 4.16(s, 2H), 2.29(s, 3H), 1.57(s, 9H)

MS(M+1): 379

### 6-Methyl-2-thioxo-1,2,3,4-tetrahydro-pyrimidine-5-carboxylic acid 4-hydroxy-benzyl ester (229)

¹H NMR(DMSO-d₆): δ 9.61(s, 1H), 7.21(d, 2H), 6.77(d, 2H), 4.44(s, 2H), 4.13(s, 2H), 4.16(s, 2H), 2.29(s, 3H)

MS(M+1): 279

### 6-Methyl-2-thioxo-1,2,3,4-tetrahydro-pyrimidine-5-carboxylic acid 1-(toluene-4-sulfonyl)-1H-pyrrol-3-ylmethyl ester (230)

¹H NMR(DMSO-d₆): δ 9.96(s, 1H), 8.93 (s, 2H), 7.83(d, 2H), 7.43(d, 2H), 7.35(s, 1H), 7.29(t, 1H), 6.34(s, 1H), 4.89(s, 2H), 3.84(s, 2H), 2.37(s, 3H), 2.06(s, 3H)

MS(M+1): 406

### 6-Methyl-2-thioxo-1,2,3,4-tetrahydro-pyrimidine-5-carboxylic acid 2-methyl-benzyl ester (231)

¹H NMR(CDCl3): δ 7.44(s, 1H), 7.31-7.20(m, 4H), 6.76(s, 1H), 5.19(s, 2H), 4.16(t, 2H), 2.35(s, 3H), 2.29(s, 3H)

MS(M+1): 277

### 6-Methyl-2-thioxo-1,2,3,4-tetrahydro-pyrimidine-5-carboxylic acid 5-methyl-thiophen-2-ylmethyl ester (232)

¹H NMR(CDCl3): δ 7.18(s, 1H), 6.87(d, 1H), 6.29(d, 1H), 6.51(s, 1H), 5.24(s, 2H), 4.15(s, 2H), 2.48(s, 3H), 2.29(s, 3H)

MS(M+1): 283

### 6-Methyl-2-thioxo-1,2,3,4-tetrahydro-pyrimidine-5-carboxylic acid 5-chloro-thiophen-2-ylmethyl ester (233)

¹H NMR(CDCl3): δ 7.34(s, 1H), 6.86(d, 1H), 6.80(d, 1H), 6.66(s, 1H), 5.21(s, 2H), 4.15(s, 2H), 2.30(s, 3H)

MS(M+1): 303

### 6-Methyl-2-thioxo-4-trifluoromethyl-1,2,3,4-tetrahydro-pyrimidine-5-carboxylic acid benzyl ester (234)

¹H NMR(DMSO-d₆): δ 10.90 (s, 1H), 9.92 (s, 1H), 7.41-7.30 (m, 5H), 5.17 (q, 2H), 4.88-4.79 (m, 1H), 2.33 (s, 3H)

MS(M+1): 331

### 6-Methyl-2-thioxo-1,2,3,4-tetrahydro-pyrimidine-5-carboxylic acid thieno[2,3-b]thiophen-2-ylmethyl ester (235)

¹H NMR(DMSO-d₆): δ 10.03 (s, 1H), 9.00 (s, 1H), 7.61 (d, 1H), 7.38 (s, 1H), 7.29 (d, 1H), 5.33 (s, 2H), 3.89 (s, 2H), 2.20 (s, 3H)

MS(M+1): 325

### 6-Methyl-2-thioxo-1,2,3,4-tetrahydro-pyrimidine-5-carboxylic acid 4-ethoxy-but-2-inyl ester (236)

¹H NMR(DMSO-d₆): δ 10.06 (s, 1H), 9.02 (s, 1H), 4.77 (t, 2H), 4.16 (t, 2H), 3.89 (s, 2H), 3.46 (q, 2H), 2.19 (s, 3H), 1.09 (t, 3H)

MS(M+1): 269

### 6-Methyl-2-thioxo-1,2,3,4-tetrahydro-pyrimidine-5-carboxylic acid 4-propoxy-but-2-inyl ester (237)

¹H NMR(DMSO-d₆): δ 10.06 (s, 1H), 9.02 (s, 1H), 4.77 (s, 2H), 4.16 (s, 2H), 3.89 (s, 2H), 3.37 (t, 2H), 2.19 (s, 3H), 1.56-1.45 (m, 2H), 0.85 (t, 3H)

MS(M+1): 283

### 6-Methyl-2-thioxo-1,2,3,4-tetrahydro-pyrimidine-5-carboxylic acid benzoxazol-2-ylmethyl ester (238)

¹H NMR(DMSO-d₆): δ 10.01 (s, 1H), 9.04 (s, 1H), 7.74 (t, 2H), 7.55-7.48 (m, 2H), 5.39 (s, 2H), 3.95 (s, 2H), 2.20 (s, 3H)

MS(M+1): 304

### 6-Methyl-2-thioxo-1,2,3,4-tetrahydro-pyrimidine-5-carboxylic acid 3-thiophen-2-yl-prop-2-inyl ester (239)

¹H NMR(CDCl₃): δ 7.30-7.27 (m, 2H), 7.20 (s, 1H), 7.0 (m, 1H), 6.68 (s, 1H), 4.98 (s, 2H), 4.21 (s, 2H), 2.32 (s, 3H)

MS(M+1): 293

### 6-Methyl-2-thioxo-1,2,3,4-tetrahydro-pyrimidine-5-carboxylic acid 3-thiophen-3-yl-prop-2-inyl ester (240)

¹H NMR(CDCl₃): δ 7.50 (d, 1H), 7.28 (m, 2H), 7.12 (d, 1H), 6.62 (s, 1H), 4.95 (s, 2H), 4.21 (s, 2H), 2.33 (s, 3H)

MS(M+1): 293

### 6-Methyl-2-thioxo-1,2,3,4-tetrahydro-pyrimidine-5-carboxylic acid 1-methyl-1H-benzoimidazol-2-ylmethyl ester (241)

¹H NMR(DMSO-d₆): δ 10.04 (s, 1H), 8.99 (s, 1H), 7.58 (dd, 2H), 7.24 (dt, 2H), 5.36 (s, 2H), 3.90 (s, 2H), 3.79 (s, 3H), 2.18 (s, 3H)

MS(M+1): 317

### 6-Methyl-2-thioxo-1,2,3,4-tetrahydro-pyrimidine-5-carboxylic acid 3-methyl-3H-benzotriazol-5-ylmethyl ester (242)

¹H NMR(DMSO-d₆): δ 9.99 (s, 1H), 8.97 (s, 1H), 8.01 (s, 1H), 7.83 (m, 1H), 7.56 [symbol] 7.38 (m, 1H), 5.25 (m, 2H), 4.29 (s, 3H), 3.92 (m, 2H), 2.18 (s, 3H) MS(M+1): 318

### 6-Methyl-2-thioxo-1,2,3,4-tetrahydro-pyrimidine-5-carboxylic acid 3-thiophen-3-yl-propyl ester (243)

¹H NMR(CDCl₃): δ 7.28 (s, 1H), 6.95 (m, 2H), 6.61 (s, 1H), 4.16 (t, 2H), 4.11 (s, 2H), 2.72 (t, 2H), 2.29 (s, 3H), 2.03 (m, 2H)

MS(M+1): 297

### 6-Methyl-2-thioxo-1,2,3,4-tetrahydro-pyrimidine-5-carboxylic acid 3-thiophen-3-yl-allyl ester (244)

¹H NMR(CDCl₃): δ 7.30 (m, 2H), 7.21 (d, 2H), 6.68 (s, 1H), 6.65 (s, 1H), 6.14 (m, 1H), 4.77 (d, 2H), 4.18 (s, 2H), 2.30 (s, 3H)

MS(M+1): 295

### 6-Methyl-2-thioxo-1,2,3,4-tetrahydro-pyrimidine-5-carboxylic acid 4allyloxy-but-2-inyl ester (245)

¹H NMR(DMSO-d₆): δ 10.04 (s, 1H), 9.00 (s, 1H), 5.90-5.80 (m, 1H), 5.25-5.14 (m, 2H), 4.76 (s, 2H), 4.17 (s, 2H), 3.96 (m, 2H), 3.88 (s, 2H), 2.18 (s, 3H)

MS(M+1): 281

### 6-Methyl-2-thioxo-1,2,3,4-tetrahydro-pyrimidine-5-carboxylic acid 4-(1-methyl-prop-2-inyloxy)-but-2-inyl ester (246)

¹H NMR(CDCl₃): δ 7.32 (s, 1H), 6.65 (s, 1H), 4.79 (s, 1H), 4.72 (s, 2H), 4.18 (s, 2H), 2.31 (s, 3H), 2.11 (s, 3H)

MS(M+1): 293

### 6-Methyl-2-thioxo-1,2,3,4-tetrahydro-pyrimidine-5-carboxylic acid 2-(2-bromo-thiophen-3-yl)-ethyl ester (247)

¹H NMR(DMSO-d₆): δ 9.94 (s, 1H), 8.94 (s, 1H), 7.55 (d, 1H), 6.98 (d, 1H), 4.23 (t, 1H), 3.82 (s, 2H), 2.85 (t, 2H), 2.09 (s, 3H)

MS(M+1): 362

### 6-Methyl-2-thioxo-1,2,3,4-tetrahydro-pyrimidine-5-carboxylic acid benzothiazol-6-ylmethyl ester (248)

¹H NMR(DMSO-d₆): δ 10.00 (s, 1H), 9.39 (s, 1H), 8.97 (s, 1H), 8.16 (s, 1H), 8.07 (d, 1H), 7.53 (d, 1H), 5.25 (s, 2H), 3.93 (s, 2H), 2.19 (s, 3H)

MS(M+1): 320

### 6-Methyl-2-thioxo-1,2,3,4-tetrahydro-pyrimidine-5-carboxylic acid 2-(2-fluoro-thiophen-3-yl)-ethyl ester (249)

¹H NMR(CDCl₃): δ 7.19 (s, 1H), 6.60 (m, 2H), 6.55 (d, 1H), 4.31 (t, 2H), 4.11 (s, 2H), 2.88 (t, 2H), 2.24 (s, 3H)

MS(M+1): 301

### 6-Methyl-2-thioxo-1,2,3,4-tetrahydro-pyrimidine-5-carboxylic acid 3-furan-2-yl-propyl ester (250)

¹H NMR(CDCl₃): δ 7.35 (s, 1H), 7.30 (d, 1H), 6.70 (s, 1H), 6.29 (d, 1H), 6.01 (dd, 1H), 4.18 (t, 2H), 4.13 (s, 2H), 2.71 (t, 2H), 2.29 (s, 3H), 2.01 (quin, 2H)

MS(M+1): 281

### 6-Methyl-2-thioxo-1,2,3,4-tetrahydro-pyrimidine-5-carboxylic acid 3-furan-2-yl-allyl ester (251)

¹H NMR(DMSO-d₆): δ 9.99 (s, 1H), 8.98 (s, 1H), 7.63 (s, 1H), 6.53 (d, 1H), 6.50-6.48 (m, 2H), 6.15 (dt, 1H), 4.70 (d, 2H), 3.93 (s, 2H), 2.20 (s, 3H)

MS(M+1): 279

### 4-Fluoromethyl-6-methyl-2-thioxo-1,2,3,4-tetrahydro-pyrimidine-5-carboxylic acid 2-thiophen-3-yl-ethyl ester (252)

¹H NMR(DMSO-d₆): δ 10.3 (s, 1H), 9.35 (s, 1H), 7.48 (dd, 1H), 7.24 (d, 1H), 7.04 (d, 1H), 4.35-4.18 (m, 3H), 4.18 (d, 1H), 4.06 (d, 1H), 2.94 (t, 2H), 2.16 (s, 3H)

MS(M+1): 315

### 4-Fluoromethyl-6-methyl-2-thioxo-1,2,3,4-tetrahydro-pyrimidine-5-carboxylic acid thiophen-3-ylmethyl ester (253)

¹H NMR(DMSO-d₆): δ 10.3 (s, 1H), 9.38 (s, 1H), 7.56-7.50 (m, 2H), 7.15-7.10 (m, 1H), 5.13 (s, 2H), 4.38-4.26 (m, 2H), 4.20-4.12 (m, 1H), 2.24 (s, 3H)

MS(M+1): 301

### 6-Methyl-2-thioxo-1,2,3,4-tetrahydro-pyrimidine-5-carboxylic acid 2-furan-3-yl-ethyl ester (254)

¹H NMR(DMSO-d₆): δ 9.93 (s, 1H), 9.27 (s, 1H), 7.56 (s, 1H), 7.48 (s, 1H), 6.40 (s, 1H), 6.29 (s, 2H), 4.18 (t, 2H), 3.86 (s, 2H), 2.70 (t, 2H), 2.26 (s, 3H)

MS(M+1): 267

### 6-Methyl-2-thioxo-1,2,3,4-tetrahydro-pyrimidine-5-carboxylic acid 2-thiophen-3-yl-1-methyl-ethyl ester (255)

¹H NMR(DMSO-d₆): δ 9.36 (s, 1H), 8.97 (s, 1H), 7.51-7.49 (m, 1H), 7.43-7.41 (m, 1H), 7.12-7.10 (m, 1H), 5.91 (q, 1H), 3.89 (s, 2H), 2.18 (s, 3H), 1.48 (d, 3H)

MS(M+1): 297

### 6-Methyl-2-thioxo-1,2,3,4-tetrahydro-pyrimidine-5-carboxylic acid 2-methyl-benzoxazol-6-ylmethyl ester (256)

¹H NMR(DMSO-d₆): δ 9.99 (s, 1H), 8.96 (s, 1H), 7.66 (s, 1H), 7.63 (d, 1H), 7.33 (d, 1H), 5.19 (s, 2H), 3.90 (s, 2H), 2.59 (s, 3H), 2.17 (s, 3H)

MS(M+1): 318

### 6-Methyl-2-thioxo-1,2,3,4-tetrahydro-pyrimidine-5-carboxylic acid 2-(3-methyl-thiophen-2-yl)-ethyl ester (257)

¹H NMR(CDCl₃): δ 7.23 (s, 1H), 7.06 (s, 1H), 6.81 (d, 1H), 6.59 (d, 1H), 4.31 (t, 2H), 4.15 (s, 2H), 3.08 (t, 2H), 2.26 (s, 3H), 2.18 (s, 3H)

MS(M+1): 297

### 6-Methyl-2-thioxo-1,2,3,4-tetrahydro-pyrimidine-5-carboxylic acid 2-(5-methyl-thiophen-2-yl)-ethyl ester (258)

¹H NMR(CDCl₃): δ 7.23 (s, 1H), 6.61 (m, 2H), 6.57 (d, 1H), 4.33 (t, 2H), 4.15 (s, 2H), 3.09 (t, 2H), 2.47 (s, 3H), 2.26 (s, 3H)

MS(M+1): 297

### 6-Methyl-2-thioxo-1,2,3,4-tetrahydro-pyrimidine-5-carboxylic acid 2-(4-fluoro-phenyl)-ethyl ester (259)

¹H NMR(DMSO-d₆): δ 9.94 (s, 1H), 8.94 (s, 1H), 7.27 (dd, 2H), 7.10 (dd, 2H), 4.23 (t, 2H), 3.82 (s, 2H), 2.89 (t, 2H), 2.10 (s, 3H)

MS(M+1): 295

### 2-Thioxo-1,2,3,4-tetrahydro-pyrimidine-5-carboxylic acid 2-thiophen-3-yl-ethyl ester (261)

¹H NMR(DMSO-d₆): δ 9.97 (s, 1H), 8.95 (s, 1H), 7.48 (dd, 1H), 7.25 (d, 1H), 7.02 (dd, 1H), 6.87 (s, 1H), 4.26 (t, 2H), 3.90 (s, 2H), 2.93 (t, 2H)

MS(M+1): 269

### 4-Fluoromethyl-6-methyl-2-thioxo-1,2,3,4-tetrahydro-pyrimidine-5-carboxylic acid 2-ethoxy-benzyl ester (263)

¹H NMR(DMSO-d₆): δ 10.3 (s, 1H), 9.34 (s, 1H), 7.35-7.27 (m, 2H), 7.01 (d, 1H), 6.93 (t, 1H), 5.12 (q, 2H), 4.35-4.25 (m, 2H), 4.21-4.12 (m, 1H), 4.05 (q, 2H), 2.23 (s, 3H), 1.31 (t, 3H)

MS(M+1): 339

### 6-Methyl-2-thioxo-1,2,3,4-tetrahydro-pyrimidine-5-carboxylic acid 5-ethyl-furan-2-ylmethyl ester (264)

¹H NMR(DMSO-d₆): δ 10.0 (s, 1H), 8.95 (s, 1H), 6.38 (d, 1H), 6.06 (d, 1H), 5.01 (s, 2H), 3.85 (s, 2H), 2.60 (q, 2H), 2.17 (s, 3H), 1.15 (t, 3H)

MS(M+1): 281

### 6-Methyl-2-thioxo-1,2,3,4-tetrahydro-pyrimidine-5-carboxylic acid 2-benzoxazol-2-yl-ethyl ester (265)

¹H NMR(DMSO-d₆): δ 9.93 (s, 1H), 8.93 (s, 1H), 7.68 (m, 2H), 7.34 (m, 2H), 4.49 (m, 2H), 3.79 (s, 2H), 3.36 (m, 2H), 2.06 (s, 3H)

MS(M+1): 318

### 6-Methyl-2-thioxo-1,2,3,4-tetrahydro-pyrimidine-5-carboxylic acid 2-(5-ethyl-thiophen-2-yl)-ethyl ester(266)

¹H NMR(CDCl₃): δ 7.20 (s, 1H), 6.60 (s, 1H), 6.34 (m, 2H), 4.34 (t, 2H), 4.15 (s, 2H), 3.10 (t, 2H), 2.81 (m, 2H), 2.29 (s, 3H), 1.29 (t, 3H)

MS(M+1): 311

### 6-Methyl-2-thioxo-1,2,3,4-tetrahydro-pyrimidine-5-carboxylic acid 2-(1,3-dimethyl-1H-thieno[2,3-c]pyrazol-5-yl)-ethyl ester (267)

¹H NMR(CDCl₃): δ 7.57 (s, 1H), 6.98 (s, 1H), 6.71 (s, 1H), 4.39 (t, 2H), 4.15 (t, 2H), 4.00 (s, 2H), 3.15 (t, 2H), 2.49 (s, 3H), 2.28 (s, 3H)

MS(M+1): 351

### 4-Fluoromethyl-6-methyl-2-thioxo-1,2,3,4-tetrahydro-pyrimidine-5-carboxylic acid 2-(2-bromo-thiophen-3-yl)-ethyl ester (268)

¹H NMR(CDCl₃): δ 7.54 (s, 1H), 7.24 (d, 1H), 7.04 (s, 1H), 6.82 (d, 1H), 4.60-4.53 (m, 1H), 4.39-4.32 (m, 2H), 4.30-4.16 (m, 2H), 2.98 (t, 2H), 2.28 (s, 3H)

MS(M+1): 394

### 4-Fluoromethyl-6-methyl-2-thioxo-1,2,3,4-tetrahydro-pyrimidine-5-carboxylic acid 3-thiophen-3-yl-allyl ester (269)

¹H NMR(CDCl₃): δ 7.64 (s, 1H), 7.30-7.20 (m, 3H), 7.12 (s, 1H), 6.69-6.66 (m, 1H), 6.17-6.09 (m, 1H) 4.79-4.74 (m. 2H), 4.68-4.63 (m, 1H), 4.43-4.39 (m, 1H), (m, 1H), 4.31-4.18 (m, 1H), 2.35 (s, 3H)

MS(M+1): 327

### 4-Fluoromethyl-6-methyl-2-thioxo-1,2,3,4-tetrahydro-pyrimidine-5-carboxylic acid 3-furan-2-yl-propyl ester (270)

¹H NMR(CDCl₃): δ 7.53 (s, 1H), 7.31 (d, 1H), 7.04 (s, 1H), 6.29 (d, 1H), 6.02 (d, 1H), 4.62-4.57 (m, 1H), 4.40-4.26 (m, 2H), 4.24-4.16 (m, 2H), 2.71 (m, 2H), 2.33 (s, 3H), 2.06-1.99 (m, 2H)

MS(M+1): 313

### 4-Fluoromethyl-2-thioxo-1,2,3,4-tetrahydro-pyrimidine-5-carboxylic acid 2-thiophen-3-yl-ethyl ester (271)

¹H NMR(CDCl₃): δ 7.67 (s, 1H), 7.31-7.19 (m, 2H), 7.04-6.96 (m, 3H), 4.62-4.56 (m, 1H), 4.46-4.28 (m, 4H), 3.01(t. 2H)

MS(M+1): 301

### 4-Benzoyloxymethyl-6-methyl-2-thioxo-1,2,3,4-tetrahydro-pyrimidine-5-carboxylic acid 2-thiophen-3-yl-ethyl ester (272)

¹H NMR(DMSO-d₆): δ 10.2 (s, 1H), 9.24 (s, 1H), 7.46 (dd, 1H), 7.35-7.26 (m, 5H), 7.20 (d, 1H), 7.01 (d, 1H), 4.43 (q, 2H), 4.30-4.17 (m, 3H), 3.26 (dd, 1H), 3.20 (dd, 1H), 2.89 (t, 2H), 2.15 (s, 3H)

MS(M+1): 403

### 6-Methyl-2-thioxo-1,2,3,4-tetrahydro-pyrimidine-5-carboxylic acid 2-(2-ethoxy-phenyl)-ethyl ester (273)

¹H NMR(DMSO-d₆): δ 9.92 (s, 1H), 8.94 (s, 1H), 7.21-7.10 (m, 2H), 6.94 (d, 1H), 6.85 (t, 1H), 4.22 (t, 2H), 4.02 (q, 2H), 3.81 (s, 2H), 2.87 (t, 2H), 2.10 (s, 3H), 1.32 (t, 3H)

MS(M+1): 321

### 4-Fluoromethyl-6-methyl-2-thioxo-1,2,3,4-tetrahydro-pyrimidine-5-carboxylic acid 2-(2-ethoxy-phenyl)-ethyl ester (274)

¹H NMR(CDCl₃): δ 8.13 (s, 1H), 7.49 (s, 1H), 7.18 (t, 1H), 7.10 (d, 1H), 6.89-6.81 (m, 2H), 4.55-4.46 (m, 1H), 4.42-4.33 (m, 2H), 4.24 (d, 1H), 4.18 (d, 1H), 4.05 (q, 2H), 3.01 (t, 2H), 2.26 (s, 3H), 1.41 (t, 3H)

MS(M+1): 353

### 4-Fluoromethyl-6-methyl-2-thioxo-1,2,3,4-tetrahydro-pyrimidine-5-carboxylic acid 2-(2-fluoro-thiophen-3-yl)-ethyl ester (1′)

¹H NMR(CDCl₃): δ 7.53 (s, 1H), 7.25-7.23 (d, 1H), 7.04 (s, 1H), 4.60-4.53 (m, 1H), 4.39-4.16 (m, 3H), 2.98-2.96 (t, 2H), 2.28 (s, 3H)

MS (M+1): 333

### 6-Methyl-2-thioxo-1,2,3,4-tetrahydro-pyrimidine-5-carboxylic acid 2-cyclopropyl-ethyl ester (2′)

¹H NMR(CDCl₃): δ 7.39 (s, 1H), 6.75 (s, 1H), 4.21 (t, 2H), 4.16 (s, 2H), 2.29 (s, 3H), 1.56 (m, 2H), 0.71 (m, 1H), 0.48 (q, 2H), 0.10 (q, 2H)

MS (M+1): 241

### 6-Methyl-2-thioxo-1,2,3,4-tetrahydro-pyrimidine-5-carboxylic acid cyclohex-3-enylmethyl ester (3′)

¹H NMR(CDCl₃): δ 7.38 (s, 1H), 6.73 (s, 1H), 5.69 (m, 2H), 4.16 (s, 2H), 4.06 (d, 2H), 2.30 (s, 3H), 2.13 (m, 2H), 1.97 (m, 1H), 1.79 (m, 2H), 1.35 (m, 1H)

MS (M+1): 267

### 6-Methyl-4-phenyl-2-thioxo-1,2,3,4-tetrahydro-pyrimidine-5-carboxylic acid 2-thiophen-3-yl-ethyl ester (4′)

¹H NMR(CDCl₃): δ 7.89 (s, 1H), 7.31-7.20 (m, 7H), 6.87 (s, 1H), 6.86 (s, 1H), 5.34 (d, 1H), 4.29 (t, 2H), 2.88 (t, 2H), 2.30 (s, 3H)

MS (M+1): 359

### 6-Methyl-2-thioxo-1,2,3,4-tetrahydro-pyrimidine-5-carboxylic acid 2-(3-methyl-thieno[2.3-c]isothiazol-5-yl)-ethyl ester (5′)

¹H NMR(DMSO-d₆): δ 9.97 (1H, s), 8.98 (1H, s), 6.91 (1H, s), 4.30 (2H, t), 3.89 (2H, s), 3.12 (2H, t), 2.49 (3H, s), 2.14 (3H, s)

MS (M+1): 354

### 6-Methyl-2-thioxo-1,2,3,4-tetrahydro-pyrimidine-4,5-dicarboxylic acid 4-ethyl ester 5-(2-thiophen-3-yl-ethyl) ester (6′)

¹H NMR(CDCl₃): δ 7.44 (s, 1H), 7.30 (m, 1H), 7.02 (d, 2H), 6.96 (m, 2H), 4.93 (d, 1H), 4.40 (t, 2H), 4.20 (q, 2H), 3.00 (t, 2H), 2.30 (s, 3H), 1.27 (t, 3H)

MS (M+1): 355

### 8-Methyl-6-thioxo-5,7-diaza-spiro[3,5non-8-en-9-carboxylic acid 2-thiophen-3-yl-ethyl ester (7′)

¹H NMR(CDCl₃): δ 7.34 (s, 1H), 7.30 (d, 1H), 7.09 (s, 1H), 7.05 (s, 1H), 6.99 (d, 1H), 4.48 (t, 2H), 3.07 (t, 2H), 2.85 (m, 2H), 2.14 (m, 2H), 2.01 (s, 3H), 1.83 (m, 2H)

MS (M+1): 323

### 6-Methyl-2-thioxo-4-trifluoromethyl-1,2,3,4-tetrahydro-pyrimidine-5-carboxylic acid 2-thiophen-3-yl-ethyl ester (8′)

¹H NMR(DMSO-d₆): δ 8.30-7.80 (br, 2H), 7.48-7.43 (m, 1H), 7.27 (br, 1H), 7.06-7.01 (m, 1H), 5.07 (q, 1H), 4.35-4.25 (m, 2H), 2.96 (t, 2H), 2.24 (s, 3H)

MS (M+1): 351

### 6-Methyl-2-thioxo-1,2,3,4-tetrahydro-pyrimidine-5-carboxylic acid 3-thiophen-2-yl-propyl ester (9′)

¹H NMR(CDCl₃): δ 7.29 (s, 1H), 7.15-7.13 (d, 1H), 6.94-6.92 (m, 1H), 6.81-6.80 (d, 1H), 6.62 (s, 1H), 4.22-4.20 (t, 2H), 4.13 (s, 2H), 2.95-2.91 (t, 2H), 2.29 (s, 3H), 2.09-2.02 (m, 2H)

MS (M+1): 297

### 4-Fluoromethyl-6-methyl-2-thioxo-1,2,3,4-tetrahydro-pyrimidine-5-carboxylic acid 3-thiophen-2-yl-propyl ester (10′)

¹H NMR(CDCl₃): δ 7.29 (s, 1H), 7.26 (s, 1H), 7.15-7.13 (d, 1H), 6.94-6.92 (m, 1H), 6.80-6.79 (d, 1H), 4.60-4.54 (m, 1H), 4.40-4.38 (m, 1H), 4.28-4.19 (m, 3H), 2.94-2.91 (t, 2H), 2.34 (s, 3H), 2.10-2.03 (m, 2H)

MS (M+1): 329

### 6-Methyl-2-thioxo-1,2,3,4-tetrahydro-pyrimidine-5-carboxylic acid 5,6-dihydro-4H-cyclopenta[c]thiophen-5-yl ester (11′)

¹H NMR(DMSO-d₆): δ 10.01 (s, 1H), 8.99 (s, 1H), 7.08 (s, 2H), 5.70-5.65 (m, 2H), 3.80 (s, 2H), 3.12-3.09 (m, 2H), 2.75-2.71 (m, 2H), 2.24 (s, 3H)

MS (M+1): 295

### 6-Methyl-2-thioxo-1,2,3,4-tetrahydro-pyrimidine-5-carboxylic acid 1,3-dichloro-5,6-dihydro-4H-cyclopenta[c]thiophen-4-yl ester (12′)

¹H NMR(DMSO-d₆): δ 10.01 (s, 1H), 8.97 (s, 1H), 5.94-5.93 (m, 1H), 3.88-3.84 (m, 2H), 2.80-2.70 (m, 2H), 2.65-2.57 (m, 1H), 2.30-2.20 (m, 1H), 2.17 (s, 3H)

MS (M+1): 364

### 7-Methyl-5-thioxo-4,6-diaza-spiro[2,5]oct-7-ene-8-carboxylic acid 2-thiophen-3-yl-ethyl ester (13′)

¹H NMR(CDCl₃): δ 7.44 (s, 1H), 7.31 (d, 1H), 7.10 (s, 1H), 7.03 (s, 1H), 6.97 (d, 1H), 4.36 (t, 2H), 3.00 (t, 2H), 2.05 (s, 2H), 1.34 (t, 2H), 0.72 (t, 2H)

MS (M+1): 309

### 6-Methyl-2-thioxo-1,2,3,4-tetrahydro-pyrimidine-5-carboxylic acid 2-(3-nitro-phenyl)-ethyl ester (14′)

¹H NMR(CDCl₃): δ 8.13 (m, 2H), 7.56-7.49 (m, 1H), 7.23 (s, 1H), 6.59 (s, 1H), 4.41 (t, 1H), 4.11 (s, 2H), 3.09 (t, 2H), 2.24 (s, 3H)

MS (M+1): 322

### 4-Fluoromethyl-6-methyl-2-thioxo-1,2,3,4-tetrahydro-pyrimidine-5-carboxylic acid 3-chloro-4-methyl-thiophen-2-ylmethyl ester (15′)

¹H NMR(DMSO-d₆): δ 10.36 (s, 1H), 9.39 (s, 1H), 7.40 (s, 1H), 5.29-5.21 (m, 2H), 4.32-4.17 (m, 3H), 2.24 (s, 3H), 2.15 (s, 3H)

MS (M+1): 350

### 4-Fluoromethyl-2-thioxo-1,2,3,4-tetrahydro-pyrimidine-5-carboxylic acid 3-thiophen-2-yl-propyl ester (16′)

¹H NMR(CDCl₃): δ 7.73 (s, 1H), 7.15-7.14 (d, 2H), 7.00 (s, 1H), 6.95-6.92 (m, 1H), 6.81-6.80 (d, 1H), 4.63-4.51 (m, 3H), 4.25-4.22 (t, 2H), 2.96-2.92 (t, 2H), 2.11-2.04 (m, 2H)

MS (M+1): 315

### 6-Methyl-2-thioxo-1,2,3,4-tetrahydro-pyrimidine-5-carboxylic acid 5-carboxy-pentyl ester (18′)

¹H NMR(DMSO-d₆): δ 11.19 (1H, br), 9.94 (1H, s), 8.93 (1H, s), 4.01 (2H, t), 3.87 (2H, s), 2.21-2.16 (5H, m), 1.60-1.28 (6H, m)

MS (M+1): 287

### 6-Methyl-2-thioxo-1,2,3,4-tetrahydro-pyrimidine-5-carboxylic acid 5-octylcarbamoyl-pentyl ester (19′)

¹H NMR(DMSO-d₆): δ 9.96 (1H, s), 9.94 (1H, s), 8.95 (1H, s), 4.0 (2H, t), 3.88 (4H, s), 3.60 (4H, s), 3.30 (9H, s), 3.00-2.98 (2H, m), 2.17 (6H, s), 2.02 (2H, t), 1.16-1.23 (17H, m), 0.85 (3H, t)

MS (M+1): 399

### 6-Methyl-2-thioxo-1,2,3,4-tetrahydro-pyrimidine-5-carboxylic acid 9-carboxy-nonyl ester (20′)

¹H NMR(DMSO-d₆): δ 11.18 (1H, br), 9.93 (1H, s), 8.93 (1H, s), 4.01 (2H, t), 3.87 (2H, s), 2.19-2.16 (5H, m), 1.57-1.46 (5H, m), 1.24 (12H, m)

MS (M+1): 343

### 6-Methyl-2-thioxo-1,2,3,4-tetrahydro-pyrimidine-5-carboxylic acid 1-chloro-5,6-dihydro-4H-cyclopenta[c]thiophen-4-yl ester (21′)

¹H NMR(DMSO-d₆): δ 9.96 (s, 1H), 8.95 (s, 1H), 7.23 (s, 1H), 5.76 (bs, 1H), 3.84 (bs, 2H), 2.75-2.68 (m, 2H), 2.65-2.55 (m, 1H), 2.35-2.30 (m, 1H), 2.17 (s, 3H)

MS (M+1): 330

### 6-Methyl-2-thioxo-1,2,3,4-tetrahydro-pyrimidine-5-carboxylic acid 2-(5-ethyl-furan-2-yl)-ethyl ester (22′)

¹H NMR(CDCl₃): δ 7.37 (br s, 1H), 6.70 (br s, 1H), 5.94 (d, 1H), 5.87 (d, 1H), 4.36 (t, 2H), 4.11 (s, 2H), 2.94 (t, 2H), 2.63-2.54 (m, 2H), 2.24 (s, 3H), 1.20 (t, 3H)

MS (M+1): 295

### 4-Fluoromethyl-6-methyl-2-thioxo-1,2,3,4-tetrahydro-pyrimidine-5-carboxylic acid 2-(5-ethyl-furan-2-yl)-ethyl ester (23′)

¹H NMR(CDCl₃): δ 7.85 (s, 1H), 7.26 (s, 1H), 5.95 (d, 1H), 5.87 (d, 1H), 4.62-4.53 (m, 1H), 4.46-4.29 (m, 3H), 4.24-4.17 (m, 1H), 2.95 (t, 2H), 2.59 (q, 2H), 2.29 (s, 3H), 1.20 (t, 3H)

MS (M+1): 327

### (R)-4-fluoromethyl-6-methyl-2-thioxo-1,2,3,4-tetrahydro-pyrimidine-5-carboxylic acid 2-thiophen-3-yl-ethyl ester (24′)

¹H NMR(DMSO-d₆): δ 10.3 (s, 1H), 9.35 (s, 1H), 7.48 (dd, 1H), 7.24 (d, 1H), 7.04 (d, 1H), 4.35-4.18 (m, 3H), 4.18 (d, 1H), 4.06 (d, 1H), 2.94 (t, 2H), 2.16 (s, 3H)

MS (M+1): 315

### (S)-4-fluoromethyl-6-methyl-2-thioxo-1,2,3,4-tetrahydro-pyrimidine-5-carboxylic acid 2-thiophen-3-yl-ethyl ester (25′)

¹H NMR(DMSO-d₆): δ 10.3 (s, 1H), 9.35 (s, 1H), 7.48 (dd, 1H), 7.24 (d, 1H), 7.04 (d, 1H), 4.35-4.18 (m, 3H), 4.18 (d, 1H), 4.06 (d, 1H), 2.94 (t, 2H), 2.16 (s, 3H)

MS (M+1): 315

### 6-Methyl-2-thioxo-1,2,3,4-tetrahydro-pyrimidine-5-carboxylic acid 2-(5-nitro-thiophen-3-yl)-ethyl ester (26′)

¹H NMR(DMSO-d₆): δ 9.95 (s, 1H), 8.96 (s, 1H), 8.09 (d, 1H), 7.78 (d, 1H), 4.28 (t, 2H), 3.82 (s, 2H), 2.93 (t, 2H), 2.10 (s, 3H)

MS (M+1): 328

### 4-Fluoromethyl-6-methyl-2-thioxo-1,2,3,4-tetrahydro-pyrimidine-5-carboxylic acid 2-(2-bromo-thiophen-3-yl)-1-methyl-ethyl ester (27′)

¹H NMR(DMSO-d₆): δ 9.99 (1H, s), 9.10 (1H, s), 7.55-7.53 (1H, m), 6.94-6.90 (1H, m), 5.08 (1H, q), 4.85-4.78 (1H, m), 4.06 (1H, d), 2.88-2.49 (2H, m), 2.35 (3H, s), 1.20 (3H, d)

MS (M+1): 408

### 4-Fluoromethyl-6-methyl-2-thioxo-1,2,3,4-tetrahydro-pyrimidine-5-carboxylic acid 2-(5-nitro-thiophen-3-yl)-ethyl ester (28′)

¹H NMR(DMSO-d₆): δ 10.26 (s, 1H), 9.34 (s, 1H), 8.10 (s, 1H), 7.79 (d, 1H), 4.36-4.28 (m, 3H), 4.20 (t, 2H), 4.07 (m, 1H), 2.96 (t, 2H), 2.15 (s, 3H)

MS (M+1): 360

### 9-Methyl-7-thioxo-6,8-diaza-spiro[4,5]dec-9-ene-10-carboxylic acid 2-thiophen-3-yl-ethyl ester (29′)

¹H NMR(CDCl₃): δ 7.46 (s, 1H), 7.29 (d, 1H), 7.02 (s, 1H), 6.97 (d, 1H), 6.65 (s, 1H), 4.41 (t, 2H), 3.02 (t, 2H), 2.12 (m, 2H), 2.03 (s, 3H), 1.76-1.67 (m, 6H)

MS (M+1): 337

### 6-Methyl-2-thioxo-1,2,3,4-tetrahydro-pyrimidine-5-carboxylic acid 5-methyl-[1,2,4]oxadiazol-3-ylmethyl ester (30′)

¹H NMR(DMSO-d₆): δ 10.12 (1H, s), 9.04 (1H, s), 5.21 (2H, s), 3.91 (2H, s), 2.60 (3H, s), 2.20 (3H, s)

MS (M+1): 269

### 4-Fluoromethyl-6-methyl-2-thioxo-1,2,3,4-tetrahydro-pyrimidine-5-carboxylic acid 3-phenyl-propyl ester (31′)

¹H NMR(CDCl₃): δ 7.60 (s, 1H), 7.31-7.16 (m, 5H), 7.06 (s, 1H), 4.56-4.50 (m, 1H), 4.39-4.36 (m, 1H), 4.27-4.15 (m, 3H), 2.72-2.68 (m, 2H), 2.33 (s, 3H), 2.05-1.98 (m, 2H)

MS (M+1): 323

### 4-Fluoromethyl-6-methyl-2-thioxo-1,2,3,4-tetrahydro-pyrimidine-5-carboxylic acid 2-(4-fluoro-phenyl)-ethyl ester (32′)

¹H NMR(CDCl₃): δ 7.77 (br s, 1H), 7.21 (br s, 1H), 7.21-7.11 (m, 2H), 7.02-6.94 (m, 2H), 4.58-4.49 (m, 1H), 4.41-4.29 (m, 2H), 4.27-4.22 (m, 1H), 4.17-4.09 (m, 1H), 2.95 (t, 2H), 2.23 (s, 3H)

MS (M+1): 327

### 4-Fluoromethyl-6-methyl-2-thioxo-1,2,3,4-tetrahydro-pyrimidine-5-carboxylic acid phenethyl ester (33′)

¹H NMR(CDCl₃): δ 8.19 (s, 1H), 7.54 (s, 1H), 7.34-7.28 (m, 2H), 7.28-7.16 (m, 3H), 4.57-4.48 (m, 1H), 4.45-4.31 (m, 2H), 4.27-4.21 (m, 1H), 4.15-4.08 (m, 1H), 2.97 (t, 2H), 2.26 (s, 3H)

MS (M+1): 309

### 4-Fluoromethyl-6-methyl-2-thioxo-1,2,3,4-tetrahydro-pyrimidine-5-carboxylic acid 4-chloro-benzyl ester (34′)

¹H NMR(CDCl₃): δ 7.69 (s, 1H), 7.36-7.27 (m, 4H), 7.14 (s, 1H), 5.19-5.12 (m, 2H), 4.66-4.60 (m, 1H), 4.38-4.24 (m, 2H), 2.33 (s, 3H)

MS (M+1): 330

### 4-Fluoromethyl-6-methyl-2-thioxo-1,2,3,4-tetrahydro-pyrimidine-5-carboxylic acid 3,5dimethyl-benzyl ester (35′)

¹H NMR(CDCl₃): δ 7.93 (s, 1H), 7.31 (s, 1H), 7.04-6.95 (m, 3H), 5.16-5.09 (m, 2H), 4.69-4.62 (m, 1H), 4.42-4.24 (m, 2H), 2.34 (s, 3H), 2.33 (s, 6H)

MS (M+1): 323

### 4-Fluoromethyl-6-methyl-2-thioxo-1,2,3,4-tetrahydro-pyrimidine-5-carboxylic acid 5-chloro-2-methoxy-benzyl ester (36′)

¹H NMR(CDCl₃): δ 7.60 (s, 1H), 7.29-7.24 (m, 2H), 7.08 (s, 1H), 6.84-6.81 (m, 1H), 5.22-5.12 (m, 2H), 4.70-4.55 (m, 1H), 4.40-4.27 (m, 2H), 3.83 (s, 3H), 2.33 (s, 3H)

MS (M+1): 360

### 4-Fluoromethyl-6-methyl-2-thioxo-1,2,3,4-tetrahydro-pyrimidine-5-carboxylic acid 2-trifluoromethyl-benzyl ester (37′)

¹H NMR(CDCl₃): δ 7.73-7.45 (m, 5H), 7.00 (s, 1H), 5.39 (s, 2H), 4.66-4.60 (m, 1H), 4.39-4.25 (m, 2H), 2.34 (s, 3H)

MS (M+1): 363

### 6-Methyl-2-thioxo-1,2,3,4-tetrahydro-pyrimidine-5-carbothioic acid S-furan-2-ylmethyl ester (38′)

¹H NMR(CDCl₃): δ 7.34 (s, 1H), 7.33 (s, 1H), 6.70 (s, 1H), 6.31-6.24 (m, 2H), 4.26 (s, 2H), 4.23 (s, 2H), 2.31 (s, 3H)

MS (M+1): 269

### 4-Fluoromethyl-6-methyl-2-thioxo-1,2,3,4-tetrahydro-pyrimidine-5-carbothioic acid S-furan-2-ylmethyl ester (39′)

¹H NMR(CDCl₃): δ 7.53 (s, 1H), 7.34 (s, 1H), 7.07 (s, 1H), 6.31-6.24 (m, 2H), 4.74-4.23 (m, 5H), 2.37 (s, 3H)

MS (M+1): 301

### 6-Methyl-2-thioxo-1,2,3,4-tetrahydro-pyrimidine-5-carboxylic acid 2-(4-methoxy-thiophen-3-yl)-ethyl ester (40′)

¹H NMR(CDCl₃): δ 7.21 (s, 1H), 6.91 (d, 1H), 6.53 (s, 1H), 6.21 (d, 1H), 4.34 (t, 2H), 4.15 (s, 2H), 3.83 (s, 3H), 2.86 (t, 2H), 2.23 (s, 3H)

MS (M+1): 313

### 4-Fluoromethyl-6-methyl-2-thioxo-1,2,3,4-tetrahydro-pyrimidine-5-carboxylic acid 2-(4-methoxy-thiophen-3-yl)-ethyl ester (41′)

¹H NMR(CDCl₃): δ 7.67 (s, 1H), 7.13 (s, 1H), 6.91 (s, 1H), 6.22 (d, 1H), 4.58 (m, 1H), 4.41-4.29 (m, 4H), 3.82 (s, 3H), 2.88 (t, 2H), 2.21 (s, 3H)

MS (M+1): 345

### 4-Fluoromethyl-6-methyl-2-thioxo-1,2,3,4-tetrahydro-pyrimidine-5-carboxylic acid 2-(2-methoxy-phenyl)-ethyl ester (42′)

¹H NMR(DMSO-d₆): δ 10.23 (s, 1H), 9.33 (s, 1H), 7.23-6.83 (m, 4H), 4.28-4.18 (m, 3H), 4.13 (m, 1H), 4.03 (m, 1H), 3.76 (s, 3H), 2.88 (t, 2H), 2.14 (s, 3H)

MS (M+1): 339

### 4-Fluoromethyl-6-methyl-2-thioxo-1,2,3,4-tetrahydro-pyrimidine-5-carboxylic acid 3-(4-methoxy-phenyl)-propyl ester (43′)

¹H NMR(DMSO-d₆): δ 10.27 (s, 1H), 9.35 (s, 1H), 7.09 (d, 2H), 6.83 (d, 2H), 4.38-4.18 (m, 3H), 4.03 (m, 2H), 3.70 (s, 3H), 2.58 (t, 2H), 2.22 (s, 3H), 1.85 (m, 2H)

MS (M+1): 353

### 4-Fluoromethyl-6-methyl-2-thioxo-1,2,3,4-tetrahydro-pyrimidine-5-carboxylic acid cyclohex-3-enylmethyl ester (44′)

¹H NMR(DMSO-d₆): δ 10.27 (s, 1H), 9.34 (s, 1H), 5.64 (m, 2H), 4.38-4.15 (m, 3H), 4.00-3.92 (m, 2H), 2.22 (s, 3H), 2.10-1.95 (m, 3H), 1.88 (m, 1H), 1.80-1.67 (m, 2H), 1.23 (m, 1H)

MS (M+1): 299

### 4-Fluoromethyl-6-methyl-2-thioxo-1,2,3,4-tetrahydro-pyrimidine-5-carboxylic acid 3-phenyl-prop-2-inyl ester (45′)

¹H NMR(DMSO-d₆): δ 10.37 (s, 1H), 9.42 (s, 1H), 7.50-7.35 (m, 5H), 4.99 (s, 2H), 4.40-4.20 (m, 3H), 2.25 (s, 3H)

MS (M+1): 319

### 4-Fluoromethyl-6-methyl-2-thioxo-1,2,3,4-tetrahydro-pyrimidine-5-carboxylic acid 2-(benzoxazol-2-ylsulfanyl)-ethyl ester (46′)

¹H NMR(DMSO-d₆): δ 10.27 (s, 1H), 9.37 (s, 1H), 7.64 (m, 2H), 7.33 (m, 2H), 4.50-4.49 (m, 2H), 4.35-4.14 (m, 3H), 3.65 (m, 2H), 2.20 (s, 3H)

MS (M+1): 382

### 6-Methyl-2-thioxo-1,2,3,4-tetrahydro-pyrimidine-5-carboxylic acid 5-methyl-isoxazol-3-ylmethyl ester (47′)

¹H NMR(CDCl₃): δ 7.44 (s, 1H), 6.74 (s, 1H), 6.01 (s, 1H), 5.20 (s, 2H), 4.17 (s, 2H), 2.43 (s, 3H), 2.31 (s, 3H)

MS (M+1): 268

### 4-Fluoromethyl-6-methyl-2-thioxo-1,2,3,4-tetrahydro-pyrimidine-5-carboxylic acid 3-(2-oxo-benzothiazol-3-yl)-propyl ester (48′)

¹H NMR(DMSO-d₆): δ 10.26 (s, 1H), 9.38 (s, 1H), 7.66 (d, 1H), 7.40-7.30 (m, 2H), 7.20 (t, 1H), 4.40-4.00 (m, 7H), 2.21 (s, 3H), 2.00 (m, 2H)

MS (M+1): 396

### 4-Fluoromethyl-6-methyl-2-thioxo-1,2,3,4-tetrahydro-pyrimidine-5-carboxylic acid 3-(benzoxazol-2-ylsulfanyl)-propyl ester (49′)

¹H NMR(DMSO-d₆): δ 10.27 (s, 1H), 9.40 (s, 1H), 7.60 (m, 2H), 7.30 (m, 2H), 4.40-4.10 (m, 5H), 3.40 (m, 2H), 2.22 (s, 3H), 2.15 (m, 2H)

MS (M+1): 396

### 4-(3H-imidazol-4-yl)-6-methyl-2-thioxo-1,2,3,4-tetrahydro-pyrimidine-5-carboxylic acid 2-thiophen-3-yl-ethyl ester (50′)

¹H NMR(DMSO-d₆): δ 10.57 (s, 1H), 9.60 (s, 1H), 8.98 (s, 1H), 7.42 (m, 2H), 7.13 (d, 1H), 6.92 (d, 1H), 5.35 (s, 1H), 4.23 (t, 2H), 2.84 (t, 2H), 2.25 (s, 3H)

MS (M+1): 349

### 4-Fluoromethyl-6-methyl-2-thioxo-1,2,3,4-tetrahydro-pyrimidine-5-carboxylic acid cyclohexylmethyl ester (51′)

¹H NMR(DMSO-d₆): δ 10.27 (s, 1H), 9.33 (s, 1H), 4.35-4.19 (m, 3H), 3.89 (m, 2H), 2.23 (s, 3H), 1.70-1.56 (m, 6H), 1.35-1.90 (m, 5H)

MS (M+1): 301

### 4-Fluoromethyl-6-methyl-2-thioxo-1,2,3,4-tetrahydro-pyrimidine-5-carboxylic acid 3-(2-fluoro-thiophen-3-yl)-propyl ester (52′)

¹H NMR(CDCl₃): δ 7.55 (s, 1H), 7.07 (s, 1H), 6.96-6.93 (d, 1H), 6.61-6.58 (d, 1H), 4.60-4.56 (m, 1H), 4.41-4.16 (m, 4H), 2.75-2.60 (m, 2H), 2.33 (s, 3H), 2.05-1.93 (m, 2H)

MS (M+1): 347

### 4-Fluoromethyl-6-methyl-2-thioxo-1,2,3,4-tetrahydro-pyrimidine-5-carboxylic acid 3-thiophen-3-yl-propyl ester (53′)

¹H NMR(CDCl₃): δ 7.82 (s, 1H), 7.27 (s, 1H), 7.25 (s, 1H), 6.96-6.92 m, 2H), 4.59-4.54 (m, 1H), 4.40-4.18 (m, 4H), 2.75-2.71 (t, 2H), 2.34 (s, 3H), 2.05-1.98 (m, 2H)

MS (M+1): 329

### 6-Methyl-2-thioxo-1,2,3,4-tetrahydro-pyrimidine-5-carboxylic acid 2-(2-thiophen-3-yl-ethoxy)-ethyl ester (54′)

¹H NMR(DMSO-d₆): δ 9.98 (s, 1H), 8.99 (s, 1H), 7.43 (m, 1H), 7.19 (d, 1H), 7.00 (m, 1H), 4.15 (t, 2H), 3.88 (s, 2H), 3.60-3.63 (m, 4H), 2.81 (t, 2H), 2.17 (s, 3H)

MS (M+1): 327

### 4-Fluoromethyl-6-methyl-2-thioxo-1,2,3,4-tetrahydro-pyrimidine-5-carboxylic acid 4-(benzothiazol-2-ylsulfanyl)-butyl ester (55′)

¹H NMR(DMSO-d₆): 10.26 (s, 1H), 9.35 (s, 1H), 8.00 (d, 1H), 7.84 (d, 1H), 7.46 (t, 1H), 7.36 (t, 1H), 4.35-4.08 (m, 5H), 3.39 (m, 2H), 2.20 (s, 3H), 1.90-1.75 (m, 4H)

MS (M+1): 427

### 4-Fluoromethyl-6-methyl-2-thioxo-1,2,3,4-tetrahydro-pyrimidine-5-carboxylic acid 3-(4-fluorophenyl)-propyl ester (56′)

¹H NMR(CDCl₃): δ 7.55 (s, 1H), 7.14 (d, 2H), 7.05 (s, 1H), 6.99 (d, 2H), 4.62 (m, 1H), 4.42 (m, 2H), 4.20 (t, 2H), 2.70 (t, 2H), 2.34 (s, 3H), 2.01 (m, 2H)

MS (M+1): 341

### 6-Fluoromethyl-2-thioxo-1,2,3,4-tetrahydro-pyrimidine-5-carboxylic acid 2-thiophen-3-yl-ethyl ester (57′)

¹H NMR(CDCl₃): δ 7.90 (br s, 1H), 7.30 (dd, 1H), 7.01 (dd, 1H), 6.98 (br s, 1H), 6.94 (dd, 1H), 5.52 (t, 1H), 5.40 (t, 1H), 4.36 (t, 2H), 4.13 (s, 2H), 2.99 (t, 3H)

MS (M+1): 301

### 4-Fluoromethyl-6-methyl-2-thioxo-1,2,3,4-tetrahydro-pyrimidine-5-carboxylic acid 3-benzo[b]thiophen-2-yl-propyl ester (58′)

¹H NMR(DMSO-d₆): δ 10.11 (s, 1H), 9.24 (s, 1H), 7.77 (d, 1H), 7.67 (d, 1H), 7.33-7.25 (m, 2H), 7.06 (s, 1H), 4.50-4.18 (m, 5H), 2.99 (t, 2H), 2.30 (s, 3H), 2.08 (t, 2H)

MS (M+1): 379

### 4-Fluoromethyl-6-methyl-2-thioxo-1,2,3,4-tetrahydro-pyrimidine-5-carboxylic acid 3-4-[(2-methoxy-ethyl)-methyl-amino]-phenyl-propyl ester (59′)

¹H NMR(DMSO-d₆): δ 10.28 (s, 1H), 9.35 (s, 1H), 6.98 (d, 2H), 6.62 (d, 2H), 4.35-4.03 (m, 5H), 3.44 (m, 4H), 3.23 (s, 3H), 2.86 (s, 3H), 2.23 (s, 3H), 1.83 (m, 2H)

MS (M+1): 410

### 4-Fluoromethyl-6-methyl-2-thioxo-1,2,3,4-tetrahydro-pyrimidine-5-carboxylic acid 2-pentafluorophenyl-ethyl ester (60′)

¹H NMR(DMSO-d₆): δ 10.31 (s, 1H), 9.40 (s, 1H), 4.30-4.03 (m, 5H), 3.07 (m, 2H), 2.16 (s, 3H)

MS (M+1): 399

### 4-Fluoromethyl-6-methyl-2-thioxo-1,2,3,4-tetrahydro-pyrimidine-5-carboxylic acid 4-thiophen-3-yl-butyl ester (61′)

¹H NMR(DMSO-d₆): δ 10.29 (s, 1H), 9.37 (s, 1H), 7.44 (m, 1H), 7.15 (d, 1H), 6.97 (d, 1H), 4.29-4.34 (m, 2H), 4.28 (t, 2H), 2.61 (t, 2H), 2.22 (s, 3H), 1.59-164 (m, 4H)

MS (M+1): 343

### 4-Fluoromethyl-6-methyl-2-thioxo-1,2,3,4-tetrahydro-pyrimidine-5-carboxylic acid [2,2]bithiophenyl-5-ylmethyl ester (62′)

¹H NMR(CDCl₃): δ 7.56 (s, 1H), 7.18-7.00 (m, 5H), 7.23 (s, 1H), 5.30 (m, 2H), 4.65 (m, 1H), 4.42-4.26 (m, 2H), 2.31 (s, 3H)

MS (M+1): 383

### 4-Fluoromethyl-6-methyl-2-thioxo-1,2,3,4-tetrahydro-pyrimidine-5-carboxylic acid 3-methylsulfanyl-propyl ester (63′)

¹H NMR(CDCl₃): δ 7.55 (s, 1H), 7.05 (s, 1H), 4.65 (m, 1H), 4.41 (m, 1H), 4.28 (m, 2H), 2.57 (t, 2H), 2.34 (s, 3H), 2.12 (s, 3H), 1.97 (m, 2H)

MS (M+1): 293

### 4-Fluoromethyl-6-methyl-2- thioxo-1,2,3,4-tetrahydro-pyrimidine-5-carboxylic acid 1-(4-methoxy-phenyl)-cyclopentylmethyl ester (64′)

¹H NMR(DMSO-d₆): δ 10.22 (s, 1H), 9.28 (s, 1H), 7.22 (d, 2H), 6.85 (d, 2H), 4.20-3.90 (m, 5H), 3.72 (s, 3H), 2.10 (s, 3H), 1.94-1.80 (m, 4H), 1.70-1.60 (m, 4H)

MS (M+1): 393

### 4-Fluoromethyl-6-methyl-2-thioxo-1,2,3,4-tetrahydro-pyrimidine-5-carboxylic acid 2-(2,3,5,6-tetrafluoro-phenyl)-ethyl ester (65′)

¹H NMR(DMSO-d₆): δ 10.28 (s, 1H), 9.37 (s, 1H), 7.44 (m, 1H), 4.30-4.03 (m, 5H), 3.00 (m, 2H), 2.16 (s, 3H)

MS (M+1): 381

### 4-Fluoromethyl-6-methyl-2-thioxo-1,2,3,4-tetrahydro-pyrimidine-5-carboxylic acid 1-(4-methoxy-phenyl)-cyclopropylmethyl ester (66′)

¹H NMR(DMSO-d₆): δ 10.23 (s, 1H), 9.30 (s, 1H), 7.21 (d, 2H), 6.85 (d, 2H), 4.30-4.05 (m, 5H), 3.71 (s, 3H), 2.15 (s, 3H), 0.89-0.80 (m, 4H)

MS (M+1): 365

### 6-Hydroxymethyl-2-thioxo-1,2,3,4-tetrahydro-pyrimidine-5-carboxylic acid 2-thiophen-3-yl-ethyl ester (67′)

¹H NMR(DMSO-d₆): δ 9.18 (s, 1H), 8.68 (s, 1H), 7.47 (dd, 1H), 7.25 (d, 1H), 7.03 (dd, 1H), 5.62 (t, 1H), 4.47 (d, 2H), 4.25 (t, 2H), 3.92 (s, 2H), 2.92 (t, 2H)

MS (M+1): 299

### 6-Methyl-4-(2-methyl-propane-2-sulfonylmethyl)-2-thioxo-1,2,3,4-tetrahydro-pyrimid ine-5-carboxylic acid 3-thiophen-3-yl-propyl ester (68′)

¹H NMR(CDCl₃): δ 8.60 (s, 1H), 7.60 (s, 1H), 7.26 (dd, 1H), 6.98 (d, 1H), 6.95 (dd, 1H), 5.10-5.05 (m, 1H), 4.25-4.18 (m, 2H), 3.36 (dd, 1H), 3.16 (d, 1H), 2.75 (t, 2H), 2.34 (s, 3H), 2.05-1.97 (m, 2H), 1.43 (s, 9H)

MS (M+1): 431

### 4-Fluoromethyl-6-methyl-2-thioxo-1,2,3,4-tetrahydro-pyrimidine-5-carboxylic acid 3,5-difluoro-benzyl ester (69′)

¹H NMR(DMSO-d₆): δ 10.35 (s, 1H), 9.39 (s, 1H), 7.22-7.11 (m, 3H), 5.13 (s, 2H), 4.40-4.20 (m, 3H), 2.25 (s, 3H)

MS (M+1): 331

### 4-Fluoromethyl-6-methyl-2-thioxo-1,2,3,4-tetrahydro-pyrimidine-5-carboxylic acid 2-(3-chloro-phenyl)-ethyl ester (70′)

¹H NMR(DMSO-d₆): 10.26 (s, 1H), 9.35 (s, 1H), 7.35-7.21 (m, 4H), 4.34-4.03 (m, 5H), 2.94 (t, 2H), 2.15 (s, 3H)

MS (M+1): 343

### 6-Methyl-2-thioxo-1,2,3,4-tetrahydro-pyrimidine-5-carboxylic acid 2,2-difluoro-2-thiophen-3-yl-ethyl ester (71′)

¹H NMR(DMSO-d₆): δ 10.08 (s, 1H), 9.02 (s, 1H), 7.95 (d, 1H), 7.70 (m, 1H), 7.28 (m, 1H), 4.67 (dd, 2H), 3.85 (s, 2H), 2.12 (s, 3H)

MS (M+1): 319

### 4-Fluoromethyl-6-methyl-2-thioxo-1,2,3,4-tetrahydro-pyrimidine-5-carboxylic acid 3-trifluoromethylsulfanyl-benzyl ester (72′)

¹H NMR(DMSO-d₆): δ 10.35 (s, 1H), 9.39 (s, 1H), 7.74-7.54 (m, 4H), 5.24-5.17 (m, 2H), 4.39-4.20 (m, 3H), 2.24 (s, 3H)

MS (M+1): 395

### 4-Fluoromethyl-6-methyl-2-thioxo-1,2,3,4-tetrahydro-pyrimidine-5-carboxylic acid 1-methyl-3-thiophen-2-yl-propyl ester (74′)

¹H NMR(CDCl₃): δ 8.14 (s, 1H), 7.55-7.49 (d, 1H), 7.15-6.80 (m, 3H), 5.12-5.08 (m, 1H), 4.67-4.27 (m, 3H), 2.93-2.86 (m, 2H), 2.37 (s, 3H), 2.10-1.92 (m, 2H), 1.33-1.26 (m, 3H)

MS (M+1): 343

### 4-Fluoromethyl-6-methyl-2-thioxo-1,2,3,4-tetrahydro-pyrimidine-5-carboxylic acid 2-(2,6-difluoro-phenyl)-ethyl ester (75′)

¹H NMR(DMSO-d₆): δ 10.27 (s, 1H), 9.36 (s, 1H), 7.35 (m, 1H), 7.07 (m, 2H), 4.30-4.00 (m, 5H), 2.99 (m, 2H), 2.13 (s, 3H)

MS (M+1): 345

### 4-Chloromethyl-6-methyl-2-thioxo-1,2,3,4-tetrahydro-pyrimidine-5-carboxylic acid 2-thiophen-3-yl-ethyl ester (76′)

¹H NMR(CDCl): δ 8.21 (s, 1H), 7.29 (m, 1H), 7.04 (m, 1H), 6.97 (m, 1H), 4.45-4.30 (m, 3H), 3.61 (ddd, 2H), 3.04 (m, 2H), 2.33 (s, 3H)

MS (M+1): 331

### 4-Fluoromethyl-6-methyl-2-thioxo-1,2,3,4-tetrahydro-pyrimidine-5-carboxylic acid 2-(2-nitro-phenyl)-ethyl ester (77′)

¹H NMR(DMSO-d₆): 10.28 (s, 1H), 9.36 (s, 1H), 7.97 (d, 1H), 7.66 (m, 2H), 7.52 (m, 2H), 4.36 (t, 2H), 4.24-4.04 (m, 3H), 3.20 (t, 2H), 2.14 (s, 3H)

MS (M+1): 354

### 4-Fluoromethyl-6-methyl-2-thioxo-1,2,3,4-tetrahydro-pyrimidine-5-carboxylic acid 5-chloro-2-nitro-benzyl ester (78′)

¹H NMR(DMSO-d₆): δ 10.40 (s, 1H), 9.44 (s, 1H), 8.14 (d, 1H), 7.71 (m, 2H), 5.43 (s, 2H), 4.37-4.18 (m, 3H), 2.24 (s, 3H)

MS (M+1): 374

### 4-Fluoromethyl-6-methyl-2-thioxo-1,2,3,4-tetrahydro-pyrimidine-5-carboxylic acid 2-chloro-4-fluoro-benzyl ester (79′)

¹H NMR(DMSO-d₆): δ 10.36 (s, 1H), 9.39 (s, 1H), 7.59-7.50 (m, 2H), 7.25 (m, 1H), 5.18 (dd, 2H), 4.33-4.17 (m, 3H), 2.24 (s, 3H)

MS (M+1): 347

### 4-Difluoromethyl-6-methyl-2-thioxo-1,2,3,4-tetrahydro-pyrimidine-5-carboxylic acid 2-thiophen-3-yl-ethyl ester (80′)

¹H NMR(DMSO-d₆): δ 10.49 (s, 1H), 9.52 (s, 1H), 7.49-7.03 (m, 3H), 5.82-5.54 (m, 1H), 4.39-4.25 (m, 3H), 2.96-2.93 (t, 2H), 2.19 (s, 3H)

MS (M+1): 333

### 4-Fluoromethyl-6-methyl-2-thioxo-1,2,3,4-tetrahydro-pyrimidine-5-carboxylic acid 5-chloro-2-methoxy-benzyl ester (81′)

¹H NMR(DMSO-d₆): δ 10.33 (s, 1H), 9.36 (s, 1H), 7.38 (dd, 1H), 7.33 (d, 1H), 7.07 (d, 1H), 5.07 (dd, 1H), 4.33-4.17 (m, 3H), 3.81 (s, 3H), 2.23 (s, 3H)

MS (M+1): 359

### 4-Fluoromethyl-6-methyl-2-thioxo-1,2,3,4-tetrahydro-pyrimidine-5-carboxylic acid 3-chloro-4-methoxy-benzyl ester (82′)

¹H NMR(DMSO-d₆): δ 10.32 (s, 1H), 9.37 (s, 1H), 7.47 (d, 1H), 7.34 (dd, 1H), 7.14 (d, 1H), 5.05 (dd, 2H), 4.33-4.16 (m, 3H), 3.85 (s, 3H), 2.23 (s, 3H)

MS (M+1): 359

### 4-Fluoromethyl-6-methyl-2-thioxo-1,2,3,4-tetrahydro-pyrimidine-5-carboxylic acid 2-chloro-5-nitro-benzyl ester (83′)

¹H NMR(DMSO-d₆): δ 10.41 (s, 1H), 9.44 (s, 1H), 8.35 (d, 1H), 8.24 (dd, 1H), 7.83 (d, 1H), 5.30 (dd, 2H), 4.37-4.18 (m, 3H), 2.26 (s, 3H)

MS (M+1): 374

### 4-Fluoromethyl-6-methyl-2-thioxo-1,2,3,4-tetrahydro-pyrimidine-5-carboxylic acid 2-(5-chloro-2-methoxy-phenyl)-ethyl ester (84′)

¹H NMR(DMSO-d₆): δ 10.26 (s, 1H), 9.37 (s, 1H), 7.25 (m, 2H), 6.97 (d, 1H), 4.28-4.05 (m, 5H), 3.77 (s, 3H), 2.89 (t, 2H), 2.16 (s, 3H)

MS (M+1): 373

### 4-Fluoromethyl-6-methyl-2-thioxo-1,2,3,4-tetrahydro-pyrimidine-5-carboxylic acid 2-(3,5-difluoro-phenyl)-ethyl ester (85′)

¹H NMR(DMSO-d₆): δ 10.27 (s, 1H), 9.36 (s, 1H), 7.07 (m, 3H), 4.37-4.03 (m, 5H), 2.96 (t, 2H), 2.15 (s, 3H)

MS (M+1): 345

### 4-Fluoromethyl-6-methyl-2-thioxo-1,2,3,4-tetrahydro-pyrimidine-5-carboxylic acid 2-(2-chloro-4-fluoro-phenyl)-ethyl ester (86′)

¹H NMR(DMSO-d₆): δ 10.28 (s, 1H), 9.36 (s, 1H), 7.43 (m, 2H), 7.18 (m, 1H), 4.32-4.05 (m, 5H), 3.05 (m, 2H), 2.16 (s, 3H)

MS (M+1): 361

### 4-Fluoromethyl-6-methyl-2-thioxo-1,2,3,4-tetrahydro-pyrimidine-5-carboxylic acid 2-(2-chloro-6-fluoro-phenyl)-ethyl ester (87′)

¹H NMR(DMSO-d₆): δ 10.29 (s, 1H), 9.38 (s, 1H), 7.34 (m, 2H), 7.20 (m, 1H), 4.33-4.03 (m, 5H), 3.11 (m, 2H), 2.15 (s, 3H)

MS (M+1): 361

### 4-Fluoromethyl-6-methyl-2-thioxo-1,2,3,4-tetrahydro-pyrimidine-5-carbothioic acid S-phenethyl ester (88′)

¹H NMR(DMSO-d₆): δ 10.52 (s, 1H), 9.56 (s, 1H), 7.30-7.18 (m, 5H), 4.50-4.18 (m, 5H), 3.15 (t, 2H), 2.83 (t, 2H), 2.26 (s, 3H)

MS (M+1): 325

### 4-Fluoromethyl-6-methyl-2-thioxo-1,2,3,4-tetrahydro-pyrimidine-5-carboxylic acid 3,5-dichloro-benzyl ester (89′)

¹H NMR(DMSO-d₆): δ 10.35 (s, 1H), 9.39 (s, 1H), 7.22-7.11 (m, 3H), 5.13 (s, 2H), 4.40-4.20 (m, 3H), 2.25 (s, 3H)

MS (M+1): 364

### 4-Fluoromethyl-6-methyl-2-thioxo-1,2,3,4-tetrahydro-pyrimidine-5-carboxylic acid 2-(3,4-difluoro-phenyl)-ethyl ester (90′)

¹H NMR(DMSO-d₆): δ 10.28 (s, 1H), 9.36 (s, 1H), 7.35 (m, 2H), 7.11 (bs, 1H), 4.28 (m, 3H), 4.16 (m, 1H), 4.04 (bs, 1H), 2.93 (t, 2H), 2.15 (s, 3H)

MS (M+1): 345

### 4-Fluoromethyl-6-methyl-2-thioxo-1,2,3,4-tetrahydro-pyrimidine-5-carboxylic acid 3,4,5-trifluoro-benzyl ester (91′)

¹H NMR(DMSO-d₆): δ 10.28 (s, 1H), 9.36 (s, 1H), 7.35 (m, 2H), 7.11 (bs, 1H), 4.28 (m, 3H), 4.16 (m, 1H), 4.04 (bs, 1H), 2.93 (t, 2H), 2.15 (s, 3H)

MS (M+1): 349

### Example 1″

### Synthesis of 4-hydroxymethyl-6-methyl-2-thioxo-1,2,3,4-tetrahydro-pyrimidine-5-carboxylic acid 2-(5-chloro-thiophen-2-yl)-ethyl ester (1″)

5-Chloro-2-thiophenethanol (2.44 g, 15 mmol) was introduced into a round bottomed flask, and dichloromethane (10 ml) was added thereto. This solution was cooled using an ice bath. Thereto were slowly added diketene (1.89 g, 22.5 mmol), and triethylamine (0.2 ml. 1.5 mmol). The resulting solution was warmed to room temperature, stirred for 3 hours, and water (10 ml) was added thereto. The organic layer was dried over MgSO₄ and distilled under reduced pressure to give 5-chloro-2-thiophenethyl acetoacetate (3.59 g) in the yield of 97%. Thus obtained 5-chloro-2-thiophenethyl acetoacetate (2.47 g, 10 mmol), 4-methoxybenzyl isothiourea hydrochloride (2.79 g, 12 mmol), 4-methoxybenzyloxyacetaldehyde (2.16 g, 12 mmol), potassium carbonate (5.52 g, 20 mmol), and ethanol (100 ml) were introduced into a round bottomed flask, and stirred at reflux for 2 hours. After completion of the reaction, ethanol was distilled under reduced pressure. To the filtrate were added water (30 ml) and ethyl acetate (50 ml). The organic layer was separated, dried, and purified by column chromatography (n-hexane/ethyl acetate=3/1, v/v) to give 6-methyl-2-(4-methoxybenzyl-thio)-1,4-dihydro-pyrimidine-4-paramethoxybenzyloxymethyl-5-carboxylic acid 2-(5-chlorothiophen)-2-yl-ethyl ester (3.53 g) in the yield of 60%. Thus obtained compound (2.93 g, 5 mmol), ethanethiol (1.85 ml, 25 mmol), trifluoroacetic acid (6 ml), and dichloromethane (6 ml) were introduced into a round bottomed flask, and stirred at room temperature for 15 hours. After completion of the reaction, dichloromethane was distilled under reduced pressure. The residue was neutralized by aqueous sodium hydrogen carbonate solution, and ethyl acetate (50 ml) was added thereto. The organic layer was separated, dried, and purified by column chromatography (n-hexane/ethyl acetate=1/1, v/v) to give the title compound (0.52 g) in the yield of 30%. When the purity of the title compound was low, it was further purified by preparative HPLC.

¹H NMR(CDCl₃): δ 7.72 (s, 1H), 7.61 (s, 1H), 6.73 (d, 1H), 6.60 (d, 1H), 4.45 (m, 1H), 4.32 (t, 2H), 3.54 (t, 2H), 3.07 (t, 2H), 2.28 (s, 1H), 2.26 (s, 3H)

MS(M+1): 347

### Preparation 3

### Synthesis of 4-methoxybenzyloxyacetaldehyde

2-Buten-1,4-diol (0.88 g, 10 mmol), sodium hydride (0.88 g, 22 mmol), and N,N′-dimethylformamide (30 ml) were introduced into a round bottomed flask, which was then stirred. 4-Methoxybenzyl chloride (3.45 g, 22 mmol) was slowly added thereto under stirring. After completion of the reaction, diethylether (100 ml) was added thereto, and the organic layer was separated, dried, and purified by column chromatography (n-hexane/ethyl acetate=3/1, v/v) to give 1,4-bis(para-methoxybenzyloxy)-2-butene (3.45 g) in a yield of 95%. Thus obtained compound was dissolved in dichloromethane (30 ml), and ozone was added thereto at −78° C. for 1 hour. After completion of the reaction, dimethylsulfide (10 mg ) was added thereto, and the resulting mixture was stirred at room temperature for 3 hours. After completion of the reaction, dichloromethane was distilled under reduced pressure. The residue was purified by column chromatography (n-hexane/ethyl acetate=3/1, v/v) to give the title compound (1.0 g) in the yield of 55%.

¹H NMR(CDCl₃): δ 9.72 (s, 1H), 7.32 (d, 2H), 6.92 (d, 2H), 4.59 (s, 2H), 4.09 (s, 2H), 3.82 (s, 3H)

MS(M+1): 181

### Preparation 4

### Synthesis of 4-methoxybenzylisothiourea hydrochloride

4-Methoxybenzyl chloride (2.35 g, 15 mmol), thiourea (1.14 g, 15 mmol), and ethanol (100 ml) were introduced into a round bottomed flask, and the mixture was stirred at reflux for 2 hours. After completion of the reaction, ethanol was distilled under reduced pressure. The residue was solidified by adding diethylether (100 ml) and dried to give the title compound (3.49 g) in the yield of 99%.

¹H NMR(DMSO-d₆): δ 9.22 (s, 3H), 7.35 (s, 1H), 6.93 (d, 1H), 4.44 (s, 2H), 3.74 (s, 3H)

MS(M+1): 233

### Example 2″

### Synthesis of 4methoxymethyl-6-methyl-2-thioxo-1,2,3,4-tetrahydro-pyrimidine-5-carboxylic acid 2-thiophen-3-yl-ethyl ester (2″)

3-Thiophen-2-yl-ethylacetoacetate (0.64 g, 3 mmol) obtained by reacting 3-thiophen-2-ethanol and diketene according to the sane procedure as Example 1, methoxyacetaldehyde dimethylacetal (0.43 g, 3.6 mmol), 4-methoxybenzyl isothiourea hydrochloride (0.83 g, 3.6 mmol), potassium carbonate (0.83 g, 6 mmol), and ethanol (30 ml) were introduced into a round bottomed flask, and the mixture was stirred at reflux for 2 hours. After completion of the reaction, ethanol was distilled under reduced pressure. To the filtrate were added water (30 ml) and ethyl acetate (50 ml), and the organic layer was separated, dried, and purified by column chromatography (n-hexane/ethyl acetate=3/1, v/v) to give 6-methyl-2-(4-methoxybenzylthio)-1,4-dihydro-pyrimidine-4-methoxymethyl-5-carboxylic acid 3-thiophen-2-yl-ethyl ester (0.80 g) in the yield of 60%. Thus obtained compound (0.45 g, 1 mmol), ethanethiol (1.85 ml, 25 mmol), trifluoroacetic acid (6 ml), and dichloromethane (6 ml) were introduced into a round bottomed flask, and the mixture was stirred at room temperature for 15 hours. After completion of the reaction, dichloromethane was distilled under reduced pressure. The residue was neutralized by aqueous sodium hydrogen carbonate solution, and ethyl acetate (50 ml) was added thereto. The organic layer was separated, dried, and purified by column chromatography (n-hexane/ethyl acetate=1/1, v/v) to give the title compound (98 mg) in the yield of 30%. When the purity of the title compound was low, it was further purified by preparative HPLC.

¹H NMR(DMSO-d₆): δ 10.10 (s, 1H), 9.18 (s, 1H), 7.47 (m, 1H), 7.24 (d, 1H), 7.20 (m, 1H), 4.23-4.33 (m, 2H), 4.18 (t, 1H), 3.18 (s, 3H), 3.03-3.17 (m, 2H), 2.93 (t, 2H), 2.14 (s, 3H)

MS(M+1): 327

Alcohols and diketenes were reacted according to the same procedure as Example 2″, and the resulting acetoacetates were reacted with 4-methoxybenzylisothioureas, and alkylated or functionalized carbonyl compounds according to the sane procedure as Example 1″ or 2″ to give the following compounds.

### 4-Hydroxymethyl-6-methyl-2-thioxo-1,2,3,4-tetrahydro-pyrimidine-5-carboxylic acid 2-thiophen-3-yl-ethyl ester (3″)

¹H NMR(CDCl₃): δ 8.26 (s, 1H), 8.24 (s, 1H), 7.28 (dd, 1H), 7.02 (dd, 1H), 6.96 (d, 1H), 4.42-4.30 (m, 3H), 3.55-3.48 (m, 2H), 3.38 (br s, 1H), 3.00 (t, 2H), 2.25 (s, 3H)

MS(M+1): 313

### 4-Hydroxymethyl-6-methyl-2-thioxo-1,2,3,4-tetrahydro-pyrimidine-5-carboxylic acid 3-thiophen-2-yl-propyl ester (4″)

¹H NMR(CDCl₃): δ 8.14 (s, 1H), 8.10 (s, 1H), 7.15-7.10 (m, 1H), 6.94-6.89 (m, 1H), 6.81-6.77 (m, 1H), 4.45-4.40 (m, 1H), 4.20 (t, 2H), 3.68-3.58 (m, 2H), 3.27-3.20 (m, 1H), 2.92 (t, 2H), 2.32 (s, 3H), 2.05 (quin, 2H)

MS(M+1): 327

### 4-Hydroxymethyl-6-methyl-2-thioxo-1,2,3,4-tetrahydro-pyrimidine-5-carboxylic acid 2-(4-fluoro-phenyl)-ethyl ester (5″)

¹H NMR(CDCl₃): δ 7.66 (br s, 1H), 7.57 (br s, 1H), 7.20-7.11 (m, 2H), 7.05-6.95 (m, 2H), 4.40-4.29 (m, 3H), 3.51 (d, 2H), 2.95 (t, 2H), 2.50 (br, 1H), 2.24 (s, 3H)

MS(M+1): 325

### 4-Hydroxymethyl-6-methyl-2-thioxo-1,2,3,4-tetrahydro-pyrimidine-5-carboxylic acid 3-thiophen-3-yl-propyl ester (6″)

¹H NMR(CDCl₃): δ 7.67 (s, 1H), 7.59 (s, 1H), 7.30-7.25 (m, 1H), 6.99-6.92 (m, 2H), 4.44-4.38 (m, 1H), 4.18 (t, 2H), 3.68-3.57 (m, 2H), 2.73 (t, 2H), 2.53 (br, 1H), 2.32 (s, 3H), 2.07-1.98 (m, 2H)

MS(M+1): 327

### 4-Hydroxymethyl-6-methyl-2-thioxo-1,2,3,4-tetrahydro-pyrimidine-5-carboxylic acid 2-(2-bromo-thiophen-3-yl)-1-methyl-ethyl ester (7″)

¹H NMR(DMSO-d₆): δ 9.97 (1H, s), 9.05 (1H, s), 7.55-7.53 (1H, m), 6.94-6.90 (1H, m), 5.08 (1H, q), 4.85-4.78 (1H, m), 4.06 (1H, d), 2.88-2.49 (2H, m), 2.49 (3H, s), 1.19 (3H, d)

MS(M+1): 406

### 4-Hydroxymethyl-6-methyl-2-thioxo-1,2,3,4-tetrahydro-pyrimidine-5-carboxylic acid benzyl ester (8″)

¹H NMR(DMSO-d₆): δ 9.97 (1H, s), 9.05 (1H, s), 7.50-7.34 (5H, m), 5.18 (2H, s), 4.85-4.78 (2H, m), 4.06 (1H, d), 2.49 (3H, s)

MS(M+1): 293

### 4-Hydroxymethyl-6-methyl-2-thioxo-1,2,3,4-tetrahydro-pyrimidine-5-carboxylic acid 2-(2-methoxy-phenyl)-ethyl ester (9″)

¹H NMR(DMSO-d₆): δ 9.95 (s, 1H), 9.03 (s, 1H), 7.21 (t, 1H), 7.15 (d, 1H), 6.96 (d, 1H), 6.86 (t, 1H), 4.80 (t, 1H), 4.19 (t, 2H), 4.02 (bs, 1H), 3.19 (m, 2H), 2.87 (t, 2H), 2.12 (s, 3H)

MS(M+1): 337

### 4-Hydroxymethyl-6-methyl-2-thioxo-1,2,3,4-tetrahydro-pyrimidine-5-carboxylic acid 2-(2-bromo-thiophen-3-yl)-ethyl ester (10″)

¹H NMR(CDCl₃): δ 7.51 (s, 1H), 7.31 (s, 1H), 7.25 (d, 1H), 6.83 (d, 1H), 4.39-4.34 (m, 3H), 3.51 (t, 2H), 2.97 (m, 2H), 2.26 (s, 3H), 2.20 (t, 1H)

MS(M+1): 392

### 4-Hydroxymethyl-6-methyl-2-thioxo-1,2,3,4-tetrahydro-pyrimidine-5-carboxylic acid 2-(2-ethoxy-phenyl)-ethyl ester (11″)

¹H NMR(CDCl₃): δ 7.64 (s, 1H), 7.59 (s, 1H), 7.23-7.16 (m, 1H), 7.12 (d, 1H), 6.90-6.82 (m, 2H), 4.43-4.30 (m, 3H), 4.04 (q, 2H), 3.48-3.42 (m, 2H), 3.04-2.95 (m, 2H), 2.35 (t, 1H), 2.24 (s, 3H), 1.41 (t, 3H)

MS(M+1): 351

### 4-Hydroxymethyl-6-methyl-2-thioxo-1,2,3,4-tetrahydro-pyrimidine-5-carboxylic acid 2-(4-dimethylamino-phenyl)-ethyl ester (12″)

¹H NMR(DMSO-d₆): δ 9.99 (s, 1H), 9.07 (s, 1H), 7.60-7.25 (m, 4H), 4.30-4.19 (m, 2H), 4.05-3.98 (m, 1H), 3.22-3.10 (m, 2H), 3.05 (s, 6H), 3.95-3.85 (m, 2H), 2.13 (s, 3H)

MS(M+1): 350

### 4-Hydroxymethyl-6-methyl-2-thioxo-1,2,3,4-tetrahydro-pyrimidine-5-carboxylic acid 3-(2-fluoro-thiophen-3-yl)-propyl ester (13″)

¹H NMR(CDCl₃): δ 7.56 (s, 1H), 7.38 (s, 1H), 6.96-6.92 (d, 1H), 6.61-6.57 (d, 1H), 4.44-4.38 (m, 1H), 4.20-4.15 (t, 2H), 3.66-3.63 (t, 2H), 2.74-2.59 (m, 2H), 2.40 (s, 1H), 2.32 (s, 3H), 2.04-1.92 (m, 2H)

MS(M+1): 345

### 4-Hydroxymethyl-6-methyl-2-thioxo-1,2,3,4-tetrahydro-pyrimidine-5-carboxylic acid 3-phenyl-propyl ester (14″)

¹H NMR(CDCl₃): δ 7.59 (s, 1H), 7.42 (s, 1H), 7.31-7.16 (m, 5H), 4.39-4.36 (m, 1H), 4.20-4.16 (t, 2H), 3.62-3.59 (t, 2H), 2.72-2.68 (t, 2H), 2.45 (s, 1H), 2.31 (s, 3H), 2.04-1.97 (m, 2H)

MS(M+1): 321

### 4-(1-Hydroxy-ethyl)-6-methyl-2-thioxo-1,2,3,4-tetrahydro-pyrimidine-5-carboxylic acid 3-thiophen-3-yl-propyl ester (15″)

¹H NMR(CDCl₃): δ 8.10 (s, 1H), 7.66 (s, 1H), 7.28-7.22 (m, 1H), 7.04-6.91 (m, 2H), 4.25-4.15 (m, 4H), 2.77-2.70 (m, 2H), 2.34 (s, 3H), 2.05-1.96 (m, 2H), 1.17 (d, 3H)

MS(M+1): 341

### 4-Hydroxymethyl-6-methyl-2-thioxo-1,2,3,4-tetrahydro-pyrimidine-5-carboxylic acid 2-(2-thiophen-3-yl-ethoxy)-ethyl ester (16″)

¹H NMR(CDCl₃): δ 7.66 (s, 1H), 7.54 (s, 1H), 7.25 (m, 1H), 7.01 (m, 1H), 6.96 (m, 1H), 4.40-4.43 (m, 1H), 4.22-4.35 (m, 2H), 3.69 (t, 2H), 3.66-3.67 (m, 2H), 3.56-3.63 (m, 2H), 2.91 (t, 1H), 2.29 (s, 3H)

MS(M+1): 357

### 4-Hydroxymethyl-6-methyl-2-thioxo-1,2,3,4-tetrahydro-pyrimidine-5-carboxylic acid 3-(3-methyl-thiophen-2-yl)-propyl ester (17″)

¹H NMR(CDCl₃): δ 7.61 (s, 1H), 7.44 (s, 1H), 7.05 (d, 1H), 6.80 (d, 1H), 4.42 (m, 1H), 4.20 (t, 2H), 3.63 (m, 2H), 2.83 (t, 2H), 2.46 (m, 1H), 2.33 (s, 3H), 2.16 (s, 3H) 2.01 (m, 2H)

MS(M+1): 341

### 4-Hydroxymethyl-6-methyl-2-thioxo-1,2,3,4-tetrahydro-pyrimidine-5-carboxylic acid 5-phenyl-pentyl ester (18″)

¹H NMR(CDCl₃): δ 8.22 (2H, s), 7.30-7.09 (5H, m), 4.43 (1H, d), 4.15-4.11 (2H, m), 3.61-3.59 (2H, m), 2.64-2.61 (2H, m), 2.29 (3H, s), 1.70-1.64 (4H, M), 1.44-1.32 (2H, m)

MS(M+1): 349

### 4-Hydroxymethyl-6-methyl-2-thioxo-1,2,3,4-tetrahydro-pyrimidine-5-carboxylic acid 3-(5-ethyl-furan-2-yl)-propyl ester (19″)

¹H NMR(CDCl₃): δ 7.58 (s, 1H), 7.33 (s, 1H), 5.89 (d, 1H), 5.86 (d, 1H), 4.43 (m, 1H), 4.20 (t, 2H), 3.63 (m, 2H), 2.67 (m, 2H), 2.59 (m, 2H), 2.38 (s, 1H), 2.29 (s, 3H), 2.00 (m, 2H), 1.25 (t, 3H)

MS(M+1): 339

### 4-Ethylcarbonyloxymethyl-6-methyl-2-thioxo-1,2,3,4-tetrahydro-pyrimidine-5-carboxylic acid 3-thiophen-2-yl-propyl ester (20″)

¹H NMR(CDCl₃): δ 8.00 (br s, 1H), 7.46 (br s, 1H), 7.13 (d, 1H), 6.92 (dd, 1H), 6.81 (d, 1H), 4.85 (br s, 1H), 4.60-4.51 (m, 1H), 4.28-4.18 (m, 2H), 4.18-4.00 (m, 2H), 3.28-3.15 (m, 2H), 2.93 (t, 2H), 2.33 (s, 3H), 2.06 (quin, 2H), 1.14 (t, 3H)

MS(M+1): 398

### 4-Acetoxymethyl-6-methyl-2-thioxo-1,2,3,4-tetrahydro-pyrimidine-5-carboxylic acid 2-thiophen-3-yl-ethyl ester (21″)

¹H NMR(DMSO-d₆): δ 10.24 (s, 1H), 9.25 (s, 1H), 7.46 (m, 1H), 7.24 (d, 1H), 7.03 (m, 1H), 4.24-4.30 (m, 3H), 3.81-3.92 (m, 2H), 2.94 (t, 2H), 2.15 (s, 3H), 1.98 (s, 3H)

MS(M+1): 355

### 4-Acetoxymethyl-6-methyl-2-thioxo-1,2,3,4-tetrahydro-pyrimidine-5-carboxylic acid 3-thiophen-3-yl-propyl ester (22″)

¹H NMR(DMSO-d₆): δ 10.26 (s, 1H), 9.27 (s, 1H), 7.45 (m, 1H), 7.17 (d, 1H), 6.99 (m, 1H), 4.32 (bs, 1H), 4.02-4.09 (m, 2H), 3.91-4.00 (m, 2H), 2.66 (t, 2H), 2.23 (s, 3H), 1.99 (s, 3H), 1.87-1.94 (m, 2H)

MS(M+1): 369

### 4-Acetoxymethyl-6-methyl-2-thioxo-1,2,3,4-tetrahydro-pyrimidine-5-carboxylic acid 3-thiophen-2-yl-propyl ester (23″)

¹H NMR(CDCl₃): δ 7.93 (s, 1H), 7.32 (s, 1H), 7.13 (m, 1H), 6.92 (m, 1H), 6.80 (d, 1H), 4.52-4.56 (m, 1H), 4.18-4.24 (m, 2H), 3.99-4.14 (m, 2H), 2.92 (t, 2H), 2.34 (s, 3H), 2.10 (s, 3H), 2.02-2.07 (m, 2H)

MS(M+1): 369

### 4-Hydroxymethyl-6-methyl-2-thioxo-1,2,3,4-tetrahydro-pyrimidine-5-carboxylic acid 3-naphthalen-1-yl-propyl ester (24″)

¹H NMR(DMSO-d₆): δ 10.02 (1H, s), 9.11 (1H, s), 8.06-7.35 (7H, m), 4.09 (1H, t), 4.16-4.08 (3H, m), 3.11 (2H, t), 2.22 (3H, s), 1.99 (2H, t)

MS(M+1): 371

### 4-Hydroxymethyl-6-methyl-2-thioxo-1,2,3,4-tetrahydro-2-pyrimidine-5-carboxylic acid 4-thiophen-3-yl-butyl ester (25″)

¹H NMR(DMSO-d₆): δ 10.02 (s, 1H), 9.09 (s, 1H), 7.44 (m, 1H), 7.15 (d, 1H), 6.98 (d, 1H), 4.89 (t, 1H), 4.02-4.10 (m, 3H), 3.24-3.29 (m, 2H), 2.61 (t, 2H), 2.19 (s, 3H), 1.60-1.64 (m, 4H)

MS(M+1): 341

### 4-Acetoxymethyl-6-methyl-2-thioxo-1,2,3,4-tetrahydro-pyrimidine-5-carboxylic acid 4-thiophen-3-yl-butyl ester (26″)

¹H NMR(DMSO-d₆): δ 10.28 (s, 1H), 9.29 (s, 1H), 7.44 (m, 1H), 7.15 (s, 1H), 6.98 (d, 1H), 4.32 (d, 1H), 3.96-4.07 (m, 3H), 3.88-3.91 (m, 1H), 2.61 (t, 2H), 2.22 (s, 3H), 1.98 (s, 3H), 1.60-1.64 (m, 4H)

MS(M+1): 383

### 4-Hydroxymethyl-6-methyl-2-thioxo-1,2,3,4-tetrahydro-pyrimidine-5-carboxylic acid 2-(4-benzyloxy-phenyl)-ethyl ester (27″)

¹H NMR(DMSO-d₆): δ 9.99 (s, 1H), 9.07 (d, 1H), 7.30-7.45 (m, 5H), 7.15 (d, 2H), 6.93 (d, 2H), 5.07 (s, 2H), 4.84 (t, 1H), 4.16-4.22 (m, 2H), 3.19-3.24 (m, 2H), 2.83 (t, 2H), 2.13 (s, 3H)

MS(M+1): 413

### 4-Hydroxymethyl-6-methyl-2-thioxo-1,2,3,4-tetrahydro-pyrimidine-5-carboxylic acid biphenyl-4-ylmethyl ester (28″)

¹H NMR(DMSO-d₆): δ 10.09 (s, 1H), 9.14 (d, 1H), 7.67 (d, 4H), 7.35-7.47 (m, 5H), 5.16 (s, 2H), 4.93 (t, 1H), 4.13 (d, 2H), 3.24-3.30 (m, 2H), 2.24 (s, 3H)

MS(M+1): 369

### 4-Hydroxymethyl-6-methyl-2-thioxo-1,2,3,4-tetrahydro-pyrimidine-5-carboxylic acid 3-methylsulfinyl-propyl ester (29″)

¹H NMR(CDCl₃): δ 7.57 (s, 1H), 7.38 (s, 1H), 4.45 (m, 1H), 4.26 (t, 2H), 3.62 (m, 2H), 2.57 (t, 2H), 2.44 (s, 1H), 2.32 (s, 3H), 2.12 (s, 3H), 1.97 (m, 2H)

MS(M+1): 291

### 6-Methyl-4-(2-methyl-propane-2-sulfonylmethyl)-2-thioxo-1,2,3,4-tetrahydro-pyrimidine-5-carboxylic acid 2-(2-ethoxy-phenyl)-ethyl ester (30″)

¹H NMR(CDCl₃): δ 8.00 (s, 1H), 7.54 (s, 1H), 7.21-7.13 (m, 2H), 6.90-6.81 (m, 2H), 5.00-4.95 (m, 1H), 4.41 (t, 2H), 4.04 (q, 2H), 3.23 (m, 2H), 3.07-2.95 (m, 2H), 2.24 (s, 3H), 1.41 (t, 3H)

MS(M+1): 455

### 4-Hydroxymethyl-6-methyl-2-thioxo-1,2,3,4-tetrahydro-pyrimidine-5-carboxylic acid 2-(3-chloro-phenyl)-ethyl ester (31″)

¹H NMR(DMSO-d₆): δ 9.98 (s, 1H), 9.06 (s, 1H), 7.34-7.20 (m, 4H), 4.80 (bs, 1H), 4.26 (m, 2H), 4.03 (m, 1H), 3.20 (m, 2H), 2.92 (m, 2H), 2.11 (s, 3H)

MS(M+1): 341

### 4-Hydroxymethyl-6-methyl-2-thioxo-1,2,3,4-tetrahydro-pyrimidine-5-carboxylic acid 2-(3-nitro-phenyl)-ethyl ester (32″)

¹H NMR(DMSO-d₆): δ 9.97 (s, 1H), 9.05 (s, 1H), 8.15 (s, 1H), 8.09 (d, 1H), 7.74 (d, 1H), 7.60 (t, 1H), 4.77 (t, 1H), 4.32 (m, 2H), 4.02 (m, 1H), 3.19-3.06 (m, 4H), 2.10 (s, 3H)

MS(M+1): 352

### 4-Hydroxymethyl-6-methyl-2-thioxo-1,2,3,4-tetrahydro-pyrimidine-5-carboxylic acid 3-trifluoromethylsulfanyl-benzyl ester (33″)

¹H NMR(DMSO-d₆): δ 10.06 (s, 1H), 9.11 (s, 1H), 7.72-7.54 (m, 4H), 5.23-5.15 (m, 2H), 4.96-4.94 (m, 1H), 4.15-4.14 (m, 2H), 2.21 (s, 3H)

MS(M+1): 393

### 4-Hydroxymethyl-6-methyl-2-thioxo-1,2,3,4-tetrahydro-pyrimidine-5-carboxylic acid 3,5-dichloro-benzyl ester (34″)

¹H NMR(DMSO-d₆): δ 10.10 (s, 1H), 9.13 (d, 1H), 7.57 (bs, 1H), 7.44 (bs, 2H), 5.12 (dd, 2H), 4.91 (t, 1H), 4.14 (m, 1H), 3.25 (m, 2H), 2.22 (s, 3H)

MS(M+1): 362

### 4-Hydroxymethyl-6-methyl-2-thioxo-1,2,3,4-tetrahydro-pyrimidine-5-carboxylic acid 2-(4-chloro-phenyl)-ethyl ester (35″)

¹H NMR(DMSO-d₆): δ 9.99 (s, 1H), 9.05 (s, 1H), 7.31 (dd, 4H), 4.82 (t, 1H), 4.24 (m, 2H), 4.01 (m, 1H), 3.18 (m, 2H), 2.90 (m, 2H), 2.11 (s, 3H)

MS(M+1): 341

### 4-Hydroxymethyl-6-methyl-2-thioxo-1,2,3,4-tetrahydro-pyrimidine-5-carboxylic acid 2-cyclopentyl-ethyl ester (36″)

¹H NMR(CDCl₃): δ 7.27 (2H, s), 7.01-6.90 (3H, m), 5.30 (1H, s), 4.04-4.03 (2H, m), 2.81 (1H, d), 2.75-2.71 (2H, m), 2.29 (3H, s), 2.02-1.91 (3H, m), 1.80-1.74 (2H, m)

MS(M+1): 299

### 4-Hydroxymethyl-6-methyl-2-thioxo-1,2,3,4-tetrahydro-pyrimidine-5-carboxylic acid 2-(3,5-dimethyl-phenyl)-ethyl ester (37″)

¹H NMR(DMSO-d₆): δ 10.00 (1H, s), 9.07 (1H, s), 4.87 (1H, t), 4.09-4.03 (3H, m), 3.30-3.24 (2H, m), 2.20 (3H, s), 1.81-1.72 (3H, m), 1.62-1.47 (6H, m), 1.12-1.07 (2H, m)

MS(M+1): 335

### 4-Hydroxymethyl-6-methyl-2-thioxo-1,2,3,4-tetrahydro-pyrimidine-5-carboxylic acid 2-(3-trifluoromethyl-phenyl)-ethyl ester (38″)

¹H NMR(DMSO-d₆): δ 9.97 (1H, s), 9.07 (1H, s), 6.83 (3H, s), 4.81 (1H, s), 4.19 (2H, d), 4.03 (1H, s), 3.20 (2H, s), 2.80 (2H, s), 2.23 (6H, s), 2.13 (3H, s)

MS(M+1): 375

### 4-Hydroxymethyl-6-methyl-2-thioxo-1,2,3,4-tetrahydro-pyrimidine-5-carboxylic acid 2-(2,6-difluoro-phenyl)-ethyl ester (39″)

¹H NMR(DMSO-d₆): δ 9.97 (s, 1H), 9.05 (s, 1H), 7.34 (m, 1H), 7.07 (m, 2H), 4.78 (bs, 1H), 4.23 (m, 2H), 4.00 (m, 1H), 3.15 (m, 2H), 2.97 (m, 2H), 2.10 (s, 3H)

MS(M+1): 343

### 4-Hydroxymethyl-6-methyl-2-thioxo-1,2,3,4-tetrahydro-pyrimidine-5-carboxylic acid 3-phenyl-prop-2-inyl ester (40″)

¹H NMR(DMSO-d₆): δ 10.11 (s, 1H), 9.14 (s, 1H), 7.45-7.35 (m, 5H), 4.98 (s, 2H), 4.91 (t, 1H), 4.13 (m, 1H), 2.23 (s, 3H)

MS(M+1): 317

### 4-Hydroxymethyl-6-methyl-2-thioxo-1,2,3,4-tetrahydro-pyrimidine-5-carboxylic acid 2-(2-chloro-phenyl)-ethyl ester (41″)

¹H NMR(DMSO-d₆): δ 9.98 (s, 1H), 9.05 (s, 1H), 7.44-7.25 (m, 4H), 4.80 (t, 1H), 4.28 (m, 2H), 4.03 (m, 1H), 3.18 (m, 2H), 3.04 (m, 2H), 2.12 (s, 3H)

MS(M+1): 341

### 4-Hydroxymethyl-6-methyl-2-thioxo-1,2,3,4-tetrahydro-pyrimidine-5-carboxylic acid 2-(3,4-dichloro-phenyl)-ethyl ester (42″)

¹H NMR(DMSO-d₆): δ 9.98 (s, 1H), 9.06 (s, 1H), 7.54 (m, 2H), 7.25 (m, 1H), 4.80 (t, 1H), 4.26 (m, 2H), 4.02 (m, 1H), 3.19 (m, 2H), 2.92 (m, 3H), 2.11 (s, 3H)

MS(M+1): 376

### 4-Hydroxymethyl-6-methyl-2-thioxo-1,2,3,4-tetrahydro-pyrimidine-5-carboxylic acid 2-(2,4-dichloro-phenyl)-ethyl ester (43″)

¹H NMR(DMSO-d₆): δ 10.00 (s, 1H), 9.06 (s, 1H), 7.60 (d, 1H), 7.38 (m, 2H), 4.81 (t, 1H), 4.27 (m, 2H), 4.03 (m, 1H), 3.18 (m, 2H), 3.04 (m, 2H), 2.13 (s, 3H)

MS(M+1): 376

### 4-Acetoxymethyl-6-methyl-2-thioxo-1,2,3,4-tetrahydro-pyrimidine-5-carboxylic acid 2-(3-chloro-phenyl)-ethyl ester (44″)

¹H NMR(DMSO-d₆): δ 10.25 (s, 1H), 9.27 (d, 1H), 7.35-7.21 (m, 4H), 4.27 (m, 3H), 4.03 (dd, 2H), 3.90-3.77 (ddd, 2H), 2.93 (t, 2H), 2.13 (s, 3H), 1.98 (s, 3H)

MS(M+1): 383

### 4-Hydroxymethyl-6-methyl-2-thioxo-1,2,3,4-tetrahydro-pyrimidine-5-carboxylic acid 2-(4-methylsulfanyl-phenyl)-ethyl ester (45″)

¹H NMR(CDCl₃): δ 7.46 (s, 1H), 7.25 (d, 2H), 7.20 (s, 1H), 7.17 (d, 2H), 4.40-4.34 (m, 3H), 3.50 (m, 2H), 2.96 (t, 2H), 2.50 (s, 3H), 2.20 (s, 3H), 2.10 (t, 1H)

MS(M+1): 353

### 4-Hydroxymethyl-6-methyl-2-thioxo-1,2,3,4-tetrahydro-pyrimidine-5-carboxylic acid 3-phenoxy-benzyl ester (46″)

¹H NMR(DMSO-d₆): δ 10.06 (s, 1H), 9.10 (s, 1H), 7.40 (m, 3H), 7.13 (m, 2H), 6.96 (m, 5H), 5.11 (dd, 2H), 4.88 (t, 1H), 4.09 (m, 1H), 3.24 (m, 2H), 2.19 (s, 3H)

MS(M+1): 385

### 4-Hydroxymethyl-6-methyl-2-thioxo-1,2,3,4-tetrahydro-pyrimidine-5-carboxylic acid 5-chloro-2-nitro-benzyl ester (47″)

¹H NMR(DMSO-d₆): δ 10.14 (s, 1H), 9.17 (s, 1H), 8.15 (d, 1H), 7.72 (m, 2H), 5.43 (dd, 2H), 4.93 (t, 1H), 4.12 (m, 1H), 3.28 (m, 2H), 2.21 (s, 3H)

MS(M+1): 372

### 4-Hydroxymethyl-6-methyl-2-thioxo-1,2,3,4-tetrahydro-pyrimidine-5-carboxylic acid 2-chloro-4-fluoro-benzyl ester (48″)

¹H NMR(DMSO-d₆): δ 10.08 (s, 1H), 9.11 (s, 1H), 7.58-7.50 (m, 2H), 7.25 (m, 1H), 5.16 (dd, 2H), 4.88 (t, 1H), 4.09 (m, 1H), 3.23 (m, 2H), 2.21 (s, 3H)

MS(M+1): 345

### 4-Ethoxycarbonyloxymethyl-6-methyl-2-thioxo-1,2,3,4-tetrahydro-pyrimidine-5-carboxylic acid 2-(3-chloro-phenyl)-ethyl ester (49″)

¹H NMR(DMSO-d₆): δ 10.25 (s, 1H), 9.30 (s, 1H), 7.35-7.21 (m, 2H), 4.28 (m, 3H), 4.12 (m, 2H), 3.85 (ddd, 2H), 2.94 (t, 2H), 2.13 (s, 3H), 1.21 (m, 3H)

MS(M+1): 413

### 4-Methanesulfonyloxymethyl-6-methyl-2-thioxo-1,2,3,4-tetrahydro-pyrimidine-5-carboxylic acid 3,5-dichloro-benzyl ester (50″)

¹H NMR(DMSO-d₆): δ 7.58 (s, 1H), 7.50 (m, 2H), 5.31 (m, 2H), 5.19 (bs, 2H), 5.02 (d, 1H), 4.35-4.17 (m, 2H), 3.15 (s, 3H), 2.28 (s, 3H)

MS(M+1): 440

### 4-Hydroxymethyl-6-methyl-2-thioxo-1,2,3,4-tetrahydro-pyrimidine-5-carboxylic acid 3-trifluoromethyl-benzyl ester (51″)

¹H NMR(DMSO-d₆): δ 9.98 (1H, s), 9.07 (1H, s), 7.63-7.54 (4H, m), 4.80 (1H, s), 4.30 (2H, d), 4.01 (1H, s), 3.17 (2H, s), 3.03-3.01 (2H, m), 2.50 (3H, s)

MS(M+1): 361

### 4-Hydroxymethyl-6-methyl-2-thioxo-1,2,3,4-tetrahydro-pyrimidine-5-carboxylic acid 1-(4-chloro-phenyl)-cyclopropylmethyl ester (52″)

¹H NMR(DMSO-d₆): δ 9.97 (s, 1H), 9.01 (s, 1H), 7.30 (q, 4H), 4.80 (bs, 1H), 4.16 (q, 2H), 4.00 (m, 1H), 3.17 (m, 2H), 2.11 (s, 3H), 0.96-0.89 (m, 4H)

MS(M+1): 367

### 4-Hydroxymethyl-6-methyl-2-thioxo-1,2,3,4-tetrahydro-pyrimidine-5-carboxylic acid 2-(2,4,6-trifluoro-phenyl)-ethyl ester (53″)

¹H NMR(DMSO-d₆): δ 9.99 (s, 1H), 9.06 (s, 1H), 7.16 (t, 2H), 4.78 (t, 1H), 4.25-4.15 (m, 2H), 3.99 (m, 1H), 3.16 (m, 2H), 2.93 (m, 2H), 2.11 (s, 3H)

MS(M+1): 361

### 4-Hydroxymethyl-6-methyl-2-thioxo-1,2,3,4-tetrahydro-pyrimidine-5-carboxylic acid 2-(5-chloro-2-nitro-phenyl)-ethyl ester (54″)

¹H NMR(DMSO-d₆): δ 9.99 (s, 1H), 9.09 (s, 1H), 8.02 (d, 1H), 7.67 (s, 1H), 7.60 (d, 1H), 4.80 (bs, 1H), 4.33 (bs, 2H), 4.01 (bs, 1H), 3.19 (m, 4H), 2.11 (s, 3H)

MS(M+1): 386

### 4-Hydroxymethyl-6-methyl-2-thioxo-1,2,3,4-tetrahydro-pyrimidine-5-carboxylic acid 2-(3-fluoro-4-methyl-phenyl)-ethyl ester (55″)

¹H NMR(CDCl₃): δ 7.28 (s, 1H), 7.63 (s, 1H), 7.13 (m, 1H), 6.89 (m, 1H), 4.40-4.33 (m, 3H), 3.53 (t, 2H), 2.95 (t, 2H), 2.59 (s, 1H), 2.27 (s, 3H)

MS(M+1): 339

### 4-Hydroxymethyl-6-methyl-2-thioxo-1,2,3,4-tetrahydro-pyrimidine-5-carboxylic acid 2-(5-chloro-2-methoxy-phenyl)-ethyl ester (56″)

¹H NMR(DMSO-d₆): δ 9.97 (s, 1H), 9.07 (s, 1H), 7.24 (m, 2H), 6.96 (d, 1H), 4.80 (t, 1H), 4.20 (t, 2H), 4.02 (m, 1H), 3.77 (s, 3H), 3.19 (m, 2H), 2.87 (m, 2H), 2.12 (s, 3H)

MS(M+1): 371

### 4-Hydroxymethyl-6-methyl-2-thioxo-1,2,3,4-tetrahydro-pyrimidine-5-carboxylic acid 2-(3,5-difluoro-phenyl)-ethyl ester (57″)

¹H NMR(DMSO-d₆): δ 9.99 (s, 1H), 9.06 (d, 1H), 7.03 (m, 3H), 4.80 (t, 1H), 4.28 (m, 2H), 4.00 (m, 1H), 3.17 (m, 2H), 2.94 (t, 2H), 2.11 (s, 3H)

MS(M+1): 343

### 4-Hydroxymethyl-6-methyl-2-thioxo-1,2,3,4-tetrahydro-pyrimidine-5-carboxylic acid 2-(2-chloro-4-fluoro-phenyl)-ethyl ester (58″)

¹H NMR(DMSO-d₆): δ 10.00 (s, 1H), 9.07 (d, 1H), 7.43 (m, 2H), 7.17 (m, 1H), 4.81 (t, 1H), 4.26 (t, 2H), 4.02 (m, 1H), 3.19 (m, 2H), 3.03 (t, 2H), 2.13 (s, 3H)

MS(M+1): 359

### 4-Hydroxymethyl-6-methyl-2-thioxo-1,2,3,4-tetrahydro-pyrimidine-5-carboxylic acid 2-(2-chloro-6-fluoro-phenyl)-ethyl ester (59″)

¹H NMR(DMSO-d₆): δ 9.99 (s, 1H), 9.07 (s, 1H), 7.33 (m, 2H), 7.20 (m, 1H), 4.78 (t, 1H), 4.31-4.19 (m, 2H), 4.02 (m, 1H), 3.16-3.07 (m, 4H), 2.11 (s, 3H)

MS(M+1): 359

### 4-Hydroxymethyl-6-methyl-2-thioxo-1,2,3,4-tetrahydro-pyrimidine-5-carboxylic acid 2-(4-ethoxy-phenyl)-ethyl ester (60″)

¹H NMR(DMSO-d₆): δ 10.08 (1H, s), 9.12 (1H, s), 7.73-7.61 (4H, m), 5.21 (2H, d), 4.89 (1H, t), 4.12 (1H, d), 3.30-3.25 (1H, m), 2.21 (3H, s)

MS(M+1): 351

### 4-Hydroxymethyl-6-methyl-2-thioxo-1,2,3,4-tetrahydro-pyrimidine-5-carboxylic acid 1-(2,4-dichloro-phenyl)-cyclopropylmethyl ester (61″)

¹H NMR(DMSO-d₆): δ 9.99 (s, 1H), 9.04 (s, 1H), 7.58 (m, 1H), 7.37 (m, 2H), 4.81 (t, 1H), 4.12 (q, 2H), 3.99 (m, 1H), 3.15 (m, 2H), 2.10 (s, 3H), 1.04 (m, 2H), 0.87 (m, 2H)

MS(M+1): 402

### 4-Hydroxymethyl-6-methyl-2-thioxo-1,2,3,4-tetrahydro-pyrimidine-5-carbothioic acid S-phenethyl ester (62″)

¹H NMR(DMSO-d₆): δ 10.26 (s, 1H), 9.31 (s, 1H), 7.30-7.15 (m, 5H), 4.22 (m, 1H), 3.29 (m, 2H), 3.12 (m, 2H), 2.80 (m, 2H), 2.22 (s, 3H)

MS(M+1): 323

### 4-Hydroxymethyl-6-methyl-2-thioxo-1,2,3,4-tetrahydro-pyrimidine-5-carboxylic acid 2-p-tolyl-ethyl ester (63″)

¹H NMR(CDCl₃): δ 7.62 (s, 1H), 7.48 (s, 1H), 7.16-7.11 (m, 4H), 4.41-4.35 (m, 3H), 3.50 (t, 2H), 2.95 (t, 2H), 2.35 (s, 3H), 2.33 (s, 1H), 2.27 (s, 3H)

MS(M+1): 321

### 4-Hydroxymethyl-6-methyl-2-thioxo-1,2,3,4-tetrahydro-pyrimidine-5-carboxylic acid 2-m-tolyl-ethyl ester (64″)

¹H NMR(CDCl₃): δ 7.40 (s, 1H), 7.23 (m, 1H), 7.13 (s, 1H), 7.05 (m, 3H), 4.43-4.34 (m, 3H), 3.50 (t, 2H), 2.96 (m, 2H), 2.36 (s, 3H), 2.26 (s, 3H)

MS(M+1): 321

### 4-Hydroxymethyl-6-methyl-2-thioxo-1,2,3,4-tetrahydro-pyrimidine-5-carboxylic acid 2-(5-ethyl-furan-2-yl)-ethyl ester (65″)

¹H NMR(DMSO-d₆): δ 10.28 (s, 1H), 9.37 (s, 1H), 6.02 (d, 1H), 5.96 (s, 1H), 4.32-4.15 (m, 4H), 4.11 (bs, 1H), 2.91 (m, 2H), 2.54 (m, 2H), 2.19 (s, 3H), 1.11 (m, 3H)

MS(M+1): 325

### 4-Hydroxymethyl-6-methyl-2-thioxo-1,2,3,4-tetrahydro-pyrimidine-5-carboxylic acid 2-(4-fluoro-3-methyl-phenyl)-ethyl ester (66″)

¹H NMR(CDCl₃): δ 7.58 (s, 1H), 7.05-6.93 (m, 3H), 4.40-4.31 (m, 3H), 3.53 (t, 2H), 2.92 (t, 2H), 2.28 (s, 3H), 2.27 (s, 3H), 2.20 (t, 1H)

MS(M+1): 339

### 4-Hydroxymethyl-6-methyl-2-thioxo-1,2,3,4-tetrahydro-pyrimidine-5-carboxylic acid 2-(4-chloro-3-trifluoromethyl-phenyl)-ethyl ester (67″)

¹H NMR(CDCl₃): δ 7.71 (s, 1H), 7.59 (s, 1H), 7.56-7.34 (m, 3H), 4.43-4.37 (m, 3H), 3.51 (t, 2H), 3.03 (t, 2H), 2.55 (s, 1H), 2.27 (s, 3H)

MS(M+1): 409

### 4-Hydroxymethyl-6-methyl-2-thioxo-1,2,3,4-tetrahydro-pyrimidine-5-carboxylic acid 2-(3,4-difluoro-phenyl)-ethyl ester (68″)

¹H NMR(DMSO-d₆): δ 9.99 (s, 1H), 9.06 (s, 1H), 7.37-7.30 (m, 2H), 7.09 (bs, 1H), 4.81 (t, 1H), 4.24 (m, 2H), 4.01 (bs, 1H), 3.20 (m, 2H), 2.91 (m, 2H), 2.11 (s, 3H)

MS(M+1): 343

### 4-Hydroxymethyl-6-methyl-2-thioxo-1,2,3,4-tetrahydro-pyrimidine-5-carboxylic acid 2-(4-methoxy-3-methyl-phenyl)-ethyl ester (69″)

¹H NMR(CDCl₃): δ 7.57 (s, 1H), 7.42 (s, 1H), 7.02 (m, 2H), 6.77 (m, 1H), 4.40-4.31 (m, 3H), 3.83 (s, 3H), 3.53 (t, 2H), 2.90 (t, 2H), 2.28 (s, 3H), 2.22 (s, 3H)

MS(M+1): 351

### 4-Hydroxymethyl-6-methyl-2-thioxo-1,2,3,4-tetrahydro-pyrimidine-5-carboxylic acid 4-(benzothiazol-2-ylsulfanyl)-butyl ester (70″)

¹H NMR(DMSO-d₆): δ 10.00 (s, 1H), 9.06 (s, 1H), 8.00 (d, 1H), 7.83 (s, 1H), 7.46 (t, 1H), 7.35 (t, 1H), 4.86 (t, 1H), 4.10 (m, 3H), 3.42 (m, 2H), 3.22 (m, 2H), 2.17 (s, 3H), 1.84-1.75 (m, 4H)

MS(M+1): 424

### 4-Hydroxymethyl-6-methyl-2-thioxo-1,2,3,4-tetrahydro-pyrimidine-5-carboxylic acid 3,5-difluoro-benzyl ester (71″)

¹H NMR(DMSO-d₆): δ 10.12 (s, 1H), 9.15 (s, 1H), 7.20 (t, 1H), 7.11 (d, 2H), 5.13 (dd, 2H), 4.93 (t, 1H), 4.14 (bs, 1H), 3.27 (m, 2H), 2.22 (s, 3H)

MS(M+1): 329

### 4-Hydroxymethyl-6-methyl-2-thioxo-1,2,3,4-tetrahydro-pyrimidine-5-carboxylic acid 2-(4-ethyl-3-fluoro-1phenyl)-ethyl ester (72″)

¹H NMR(DMSO-d₆): δ 9.98 (s, 1H), 9.07 (s, 1H), 7.19 (m, 1H), 7.00 (m, 2H), 4.82 (t, 1H), 4.23 (bs, 2H), 4.01 (bs, 1H), 3.17 (bs, 2H), 2.87 (t, 2H), 2.57 (q, 2H), 2.10 (s, 3H), 1.13 (t, 3H)

MS(M+1): 353

### 4-(2-Amino-propionyloxymethyl)-6-methyl-2-thioxo-1,2,3,4-tetrahydro-pyrimidine-5-carboxylic acid 2-(3-trifluoromethyl-phenyl)-ethyl ester (73″)

¹H NMR(DMSO-d₆): δ 9.98 (1H, s), 9.06 (1H, s), 7.14 (1H, d), 6.86 (1H, d), 4.81 (1H, t), 4.20 (2H, t), 4.04-3.96 (3H, m), 3.21 (2H, d), 2.82 (2H, t), 2.13 (3H, s), 1.31 (3H, t)

MS(M+1): 446

### 4-Hydroxymethyl-6-methyl-2-thioxo-1,2,3,4-tetrahydro-pyrimidine-5-carboxylic acid 2-(3-chloro-4-methyl-phenyl)-ethyl ester (74″)

¹H NMR(CDCl₃): δ 7.47 (s, 1H), 7.29 (s, 1H), 7.20 (m, 2H), 7.01 (m, 1H), 4.40-4.33 (m, 3H), 3.54 (t, 2H), 2.94 (t, 2H), 2.37 (s, 3H), 2.35 (s, 1H), 2.27 (s, 3H)

MS(M+1): 355

### 4-Hydroxymethyl-6-methyl-2-thioxo-1,2,3,4-tetrahydro-pyrimidine-5-carboxylic acid 2-(3-trifluoromethoxy-phenyl)-ethyl ester (75″)

¹H NMR(DMSO-d₆): δ 10.37 (1H, s), 9.36 (1H, s), 7.45-7.20 (4H, m), 4.38-4.32 (3H, m), 4.26-4.22 (1H, m), 4.13-4.09 (1H, m), 3.00 (2H, t), 2.10 (3H, s)

MS(M+1): 391

### 4-(2-Benzoyloxy-ethoxycarbonyloxymethyl)-6-methyl-2-thioxo-1,2,3,4-tetrahydro-pyrimidine-5-carboxylic acid 2-(3-chloro-phenyl)-ethyl ester (76″)

¹H NMR(DMSO-d₆): δ 9.98 (s, 1H), 9.06 (s, 1H), 7.95 (m, 2H), 7.64 (m, 1H), 7.50 (m, 2H), 7.34-7.20 (m, 4H), 4.52 (m, 4H), 4.26 (m, 2H), 4.20 (m, 2H), 4.03 (m, 1H), 2.92 (m, 2H), 2.11 (s, 3H)

MS (M+1): 534

### 4-(2,2-Dimethyl-[3,1]dioxolan-4-ylmethoxycarbonyloxymethyl)-6-methyl-2-thioxo-1,2,3,4-tetrahydro-pyrimidine-5-carboxylic acid 2-(3-nitro-phenyl)-ethyl ester (77″)

¹H NMR(DMSO-d₆): δ 9.97 (s, 1H), 9.05 (s, 1H), 8.15 (s, 1H), 8.09 (d, 1H), 7.74 (d, 1H), 7.60 (t, 1H), 4.32 (m, 4H), 4.18 (m, 3H), 4.02 (m, 2H), 3.78 (m, 1H), 3.10 (m, 2H), 2.10 (s, 3H), 1.31 (s, 3H), 1.26 (s, 3H)

MS (M+1): 510

### 6-Methyl-4-(5-methyl-2-oxo-[1,3]dioxol-4-ylmethoxycarbonyloxymethyl)-2-thioxo-1,2,3,4-tetrahydro-pyrimidine-5-carboxylic acid 2-(3-chloro-phenyl)-ethyl ester (78″)

¹H NMR(DMSO-d₆): δ 9.98 (s, 1H), 9.06 (s, 1H), 7.34-7.20 (m, 4H), 5.10 (s, 2H), 4.26 (m, 2H), 4.20 (m, 2H), 4.03 (m, 1H), 2.92 (m, 2H), 2.17 (s, 3H), 2.11 (s, 3H)

MS (M+1): 497

### 4-(2-Acetylsulfanyl-ethoxycarbonyloxymethyl)-6-methyl-2-thioxo-1,2,3,4-tetrahydro-pyrimidine-5-carboxylic acid 2-(3-nitro-phenyl)-ethyl ester (79″)

¹H NMR(DMSO-d₆): δ 9.97 (s, 1H), 9.05 (s, 1H), 8.15 (s, 1H), 8.09 (d, 1H), 7.74 (d, 1H), 7.60 (t, 1H), 4.32 (m, 2H), 4.27 (t, 2H), 4.20 (m, 2H), 4.02 (m, 1H), 3.19-3.06 (m, 4H), 2.32 (s, 3H), 2.10 (s, 3H)

MS (M+1): 498

### 4-(2-Methoxycarbonylamino-ethoxycarbonyloxymethyl)-6-methyl-2-thioxo-1,2,3,4-tetrahydro-pyrimidine-5-carboxylic acid 2-(3-chloro-phenyl)-ethyl ester (80″)

¹H NMR(DMSO-d₆): δ 9.98 (s, 1H), 9.06 (s, 1H), 7.34-7.20 (m, 5H), 4.26 (m, 2H), 4.19 (m, 4H), 4.03 (m, 1H), 3.52 (s, 3H), 3.29 (m, 2H), 2.92 (m, 2H), 2.11 (s, 3H)

MS (M+1): 486

### 4-[3-(2-Acetoxy-phenyl)-propionyloxymethyl]-6-methyl-2-thioxo-1,2,3,4-tetrahydro-pyrimidine-5-carboxylic acid 2-(3-nitro-phenyl)-ethyl ester (81″)

¹H NMR(DMSO-d₆): δ 9.97 (s, 1H), 9.05 (s, 1H), 8.15 (s, 1H), 8.09 (d, 1H), 7.74 (d, 1H), 7.60 (t, 1H), 7.25-7.00 (m, 4H), 4.32 (m, 2H), 4.12 (m, 2H), 4.02 (m, 1H), 3.43 (t, 2H), 3.10 (m, 2H), 2.95 (t, 2H), 2.32 (s, 3H), 2.10 (s, 3H)

MS (M+1): 542

### 4-(2-Methoxycarbonyl-2-methyl-propoxycarbonyloxymethyl)-6-methyl-2-thioxo-1,2,3,4-tetrahydro-pyrimidine-5-carboxylic acid 2-(3-chloro-phenyl)-ethyl ester (82″)

¹H NMR(DMSO-d₆): δ 9.98 (s, 1H), 9.06 (s, 1H), 7.34-7.20 (m, 4H), 4.26 (m, 2H), 4.22 (s, 2H), 4.18 (m, 2H), 4.03 (m, 1H), 3.63 (s, 3H), 2.92 (m, 2H), 2.11 (s, 3H), 1.22 (s, 6H)

MS (M+1): 500

### 4-Ethoxycarbonylmethoxycarbonyloxymethyl-6-methyl-2-thioxo-1,2,3,4-tetrahydro-pyridine-5-carboxylic acid 2-(3-nitro-phenyl)-ethyl ester (83″)

¹H NMR(DMSO-d₆): δ 9.97 (s, 1H), 9.05 (s, 1H), 8.15 (s, 1H), 8.09 (d, 1H), 7.74 (d, 1H), 7.60 (t, 1H), 4.81 (s, 2H), 4.32 (m, 2H), 4.22 (m, 2H), 4.15 (q, 2H), 4.02 (m, 1H), 3.10 (m, 2H), 2.10 (s, 3H), 1.20 (t, 3H)

MS (M+1): 482

### 4-(2-Methanesulfonyl-ethoxycarbonyloxymethyl)-6-methyl-2-thioxo-1,2,3,4-tetrahydro-pyrimidine-5-carboxylic acid 2-(3-chloro-phenyl)-ethyl ester (84″)

¹H NMR(DMSO-d₆): δ 9.98 (s, 1H), 9.06 (s, 1H), 7.34-7.20 (m, 4H), 4.54 (t, 2H), 4.26 (m, 2H), 4.18 (m, 2H), 4.03 (m, 1H), 3.56 (t, 2H), 3.05 (s, 3H), 2.92 (m, 2H), 2.11 (s, 3H)

MS (M+1): 492

### 4-(2-Acetylamino-ethylcarbamoyloxymethyl)-6-methyl-2-thioxo-1,2,3,4-tetrahydro-pyrimidine-5-carboxylic acid 2-(3-nitro-phenyl)-ethyl ester (85″)

¹H NMR(DMSO-d₆): δ 9.97 (s, 1H), 9.05 (s, 1H), 8.93 (s, 1H), 8.15 (s, 1H), 8.09 (d, 1H), 7.85 (s, 1H), 7.74 (d, 1H), 7.60 (t, 1H), 4.32 (m, 2H), 4.18 (m, 2H), 4.02 (m, 1H), 3.18 (m, 4H), 3.10 (m, 2H), 2.10 (s, 3H), 1.79 (s, 3H)

MS (M+1): 480

### 4-(2-Dimethylamino-ethylcarbamoyloxymethyl)-6-methyl-2-thioxo-1,2,3,4-tetrahydro-pyrimidine-5-carboxylic acid 2-(3-chloro-phenyl)-ethyl ester trifluoroacetic acid salt (86″)

¹H NMR(DMSO-d₆): δ 9.98 (s, 1H), 9.30 (bs, 1H), 9.11 (bt, 1H), 9.06 (s, 1H), 7.34-7.20 (m, 4H), 4.26 (m, 2H), 4.18 (m, 2H), 4.03 (m, 1H), 3.54 (m, 2H), 3.23 (m, 2H), 2.92 (m, 2H), 2.83 (s, 6H), 2.11 (s, 3H)

MS (M+1): 456

### 4-(2-Acetoxy-ethylcarbamoyloxymethyl)-6-methyl-2-thioxo-1,2,3,4-tetrahydro-pyrimidine-5-carboxylic acid 2-(3-nitro-phenyl)-ethyl ester (87″)

¹H NMR(DMSO-d₆): δ 9.97 (s, 1H), 9.10 (bs, 1H), 9.05 (s, 1H), 8.15 (s, 1H), 8.09 (d, 1H), 7.74 (d, 1H), 7.60 (t, 1H), 4.32 (m, 2H), 4.18 (m, 2H), 4.09 (t, 2H), 3.39 (m, 2H), 4.02 (m, 1H), 3.10 (m, 2H), 2.15 (s, 3H), 2.00 (s, 3H)

MS (M+1): 481

### 6-Methyl-4-(2-oxo-[1,3]dioxolan-4-ylmethoxycarbonyloxymethyl)-2-thioxo-1,2,3,4-tetrahydro-pyrimidine-5-carboxylic acid 2-(3-chloro-phenyl)-ethyl ester (88″)

¹H NMR(DMSO-d₆): δ 9.98 (s, 1H), 9.06 (s, 1H), 7.34-7.20 (m, 4H), 5.08 (m, 1H), 4.26-4.18 (m, 8H), 4.03 (m, 1H), 2.92 (m, 2H), 2.11 (s, 3H)

MS (M+1): 486

### 4-Benzoyloxymethyl-6-methyl-2-thioxo-1,2,3,4-tetrahydro-pyrimidine-5-carboxylic acid 2-(3-nitro-phenyl)-ethyl ester (89″)

¹H NMR(DMSO-d₆): δ 9.97 (s, 1H), 9.05 (s, 1H), 8.15 (s, 1H), 8.09 (d, 1H), 7.74 (d, 1H), 7.60 (m, 3H), 7.53 (m, 1H), 7.44 (m, 2H), 4.32 (m, 2H), 4.18 (m, 2H), 4.02 (m, 1H), 3.10 (m, 2H), 2.10 (s, 3H)

MS (M+1): 456

### Terephthalic acid mono-6-methyl-5-[2-(3-nitro-phenyl)-ethoxycarbonyl]-2-thioxo-1,2,3,4-tetrahydro-pyrimidin-4-ylmethyl ester (90″)

¹H NMR(DMSO-d₆): δ 9.97 (s, 1H), 9.05 (s, 1H), 8.15 (s, 1H), 8.09 (d, 1H), 8.04 (s, 1H), 7.96 (d, 2H), 7.78 (d, 1H), 7.70 (d, 2H), 7.60 (t, 1H), 4.32 (m, 2H), 4.18 (m, 2H), 4.02 (m, 1H), 3.10 (m, 2H), 2.10 (s, 3H)

MS (M+1): 500

### 4-Hydroxymethyl-6-methyl-2-thioxo-1,2,3,4-tetrahydro-pyrimidine-5-carboxylic acid 2-(4-diethylamino-3-nitro-phenyl)-ethyl ester (91″)

¹H NMR(DMSO-d₆): δ 9.98(s, 1H), 9.06(s, 1H), 7.59(s, 1H), 7.42(d, 1H), 7.29(d, 1H), 4.81(t, 1H), 4.25(m, 2H), 4.03(t, 1H), 3.18(m, 2H), 3.00(q, 4H), 2.91(t, 2H), 2.10(s, 3H), 0.94(t, 6H)

MS(M+1): 423

### 4-Hydroxymethyl-6-methyl-2-thioxo-1,2,3,4-tetrahydro-pyrimidine-5-carboxylic acid 2-[4-(ethyl-propyl-amino)-3-nitro-phenyl]-ethyl ester (92″)

¹H NMR(DMSO-d₆): δ 9.97(s, 1H), 9.05(s, 1H), 7.59(s, 1H), 7.43(d, 1H), 7.30(d, 1H), 4.70(t, 1H), 4.26(m, 2H), 4.03(t, 1H), 3.18(m, 2H), 3.00(m, 4H), 2.91(t, 2H), 2.11(s, 3H), 0.94(m, 8H)

MS(M+1): 437

### 4-Hydroxymethyl-6-methyl-2-thioxo-1,2,3,4-tetrahydro-pyrimidine-5-carboxylic acid 2-(4-dipropylamino-3-nitro-phenyl)-ethyl ester (93″)

¹H NMR(DMSO-d₆): δ 9.98(s, 1H), 9.06(s, 1H), 7.59(s, 1H), 7.43(d, 1H), 7.30(d, 1H), 4.80(t, 1H), 4.26(m, 2H), 4.03(t, 1H), 3.18(m, 2H), 3.04(m, 4H), 2.91(t, 2H), 2.11(s, 3H), 0.94(m, 10H)

MS(M+1): 451

### 4-Hydroxymethyl-6-methyl-2-thioxo-1,2,3,4-tetrahydro-pyrimidine-5-carboxylic acid 2-[4-(cyclopropyl-ethyl-amino)-3-nitro-phenyl]-ethyl ester (94″)

¹H NMR(DMSO-d₆): δ 9.98(s, 1H), 9.06(s, 1H), 7.59(s, 1H), 7.43(d, 1H), 7.30(d, 1H), 4.80(t, 1H), 4.26(m, 2H), 4.03(t, 1H), 3.18(m, 2H), 3.04(q, 2H), 2.91(t, 2H), 2.11(s, 3H), 1.59(m, 1H), 1.02(t, 3H), 0.61(m, 2H), 0.32(m, 2H)

MS(M+1): 435

### 4-Hydroxymethyl-6-methyl-2-thioxo-1,2,3,4-tetrahydro-pyrimidine-5-carboxylic acid 2-[4-(cyclopropyl-propyl-amino)-3-nitro-phenyl]-ethyl ester (95″)

¹H NMR(DMSO-d₆): δ 9.96(s, 1H), 9.07(s, 1H), 7.60(s, 1H), 7.47(d, 1H), 7.33(d, 1H), 4.50(t, 1H), 4.27(m, 2H), 4.02(t, 1H), 3.19(m, 2H), 3.06(q, 2H), 2.92(t, 2H), 2.10(s, 3H), 1.62(m, 1H), 1.00(m, 5H), 0.62(m, 2H), 0.34(m, 2H)

MS(M+1): 449

### 4-Hydroxymethyl-6-methyl-2-thioxo-1,2,3,4-tetrahydro-pyrimidine-5-carboxylic acid 2-[4-(allyl-methyl-amino)-3-nitro-phenyl]-ethyl ester (96″)

¹H NMR(DMSO-d₆): δ 9.99(s, 1H), 9.17(s, 1H), 7.72(s, 1H), 7.54(d, 1H), 7.13(d, 1H), 5.82(m, 1H), 5.16(m, 2H), 4.40(t, 2H), 4.02(m, 2H), 4.00(t, 1H), 3.16(m, 2H), 2.92(t, 2H), 2.90(s, 3H), 2.10(s, 3H),

MS(M+1): 449

### 4-Hydroxymethyl-6-methyl-2-thioxo-1,2,3,4-tetrahydro-pyrimidine-5-carboxylic acid 2-[4-(allyl-ethyl-amino)-3-nitro-phenyl]-ethyl ester (97″)

¹H NMR(DMSO-d₆): δ 9.96(s, 1H), 9.21(s, 1H), 7.70(s, 1H), 7.54(d, 1H), 7.11(d, 1H), 5.82(m, 1H), 5.16(m, 2H), 4.41(t, 2H), 4.01(m, 2H), 3.98(t, 1H), 3.16(m, 2H), 2.92(t, 2H), 2.89(q, 3H), 2.10(s, 3H), 1.02(t, 3H)

MS(M+1): 435

### 4-Hydroxymethyl-6-methyl-2-thioxo-1,2,3,4-tetrahydro-pyrimidine-5-carboxylic acid 2-[4-(cyclopropylmethyl-methyl-amino)-3-nitrophenyl]-ethyl ester (98″)

¹H NMR(DMSO-d₆): δ 9.90(s, 1H), 9.24(s, 1H), 7.78(s, 1H), 7.52(d, 1H), 7.18(d, 1H), 4.45(t, 2H), 3.91(t, 1H), 3.79(m, 2H), 3.32(t, 1H), 3.21(d, 2H), 2.90(t, 2H), 2.84(s, 3H), 2.12(s, 3H), 0.48(m, 1H), 0.24(m, 4H)

MS(M+1): 435

### 4-Hydroxymethyl-6-methyl-2-thioxo-1,2,3,4-tetrahydro-pyrimidine-5-carboxylic acid 2-(3-nitro-4-pyrrolidin-1-yl-phenyl)-ethyl ester (99″)

¹H NMR(CDCl₃): δ 7.71(s, 1H), 7.62(s, 1H), 7.37(s, 1H), 7.25(d, 1H), 6.91(d, 1H), 4.36(m, 3H), 3.55(d, 2H), 3.24(m, 4H), 2.94(t, 2H), 2.32(s, 3H), 2.01(m, 4H)

MS(M+1): 421

### 4-Hydroxymethyl-6-methyl-2-thioxo-1,2,3,4-tetrahydro-pyrimidine-5-carboxylic acid 2-(3-nitro-4-piperidin-1-yl-phenyl)-ethyl ester (100″)

¹H NMR(CDCl₃): δ 7.35(s, 1H), 7.24(d, 1H), 6.95(d, 1H), 4.32(t, 2H), 3.55(d, 2H), 3.03(m, 4H), 2.91(t, 2H), 2.26(s, 3H), 1.78(m, 6H)

MS(M+1): 435

### 4-Hydroxymethyl-6-methyl-2-thioxo-1,2,3,4-tetrahydro-pyrimidine-5-carboxylic acid 2-[4-(2,5-dihydro-pyrrol-1-yl)-3-nitro-phenyl]-ethyl ester (101″)

¹H NMR(CDCl₃): δ 7.45(s, 1H), 7.28(d, 1H), 6.97(d, 1H), 5.79(m, 2H), 4.40(t, 2H), 4.03(m, 4H), 3.55(d, 2H), 2.98(t, 2H), 2.31(s, 3H)

MS(M+1): 419

Experiment

Inhibitory Activity of HCV

Plasma samples from 300 persons positive to the hepatitis C antibody test were screened by antibody diagnosis kit produced by LG life Sciences Ltd. (LG HCD3.0 TMB) to select 40 plasma samples having high titer. Replicon cell line and Huh-7 cell line each were cultured in a 96-well cell culture plate, fixed by methanol/acetone (1:1) mixture, and blocked by 3% skimmed milk. The 40 plasma sanples selected above were 100-fold diluted and aided thereto, and the bound antibodies were probed by anti-Human goat antibody (Sigma) to which Horse Radish Peroxidase is attached. As a result, 12 plasma samples having low specificity to Huh-7 cell line, but high specificity to replicon cell line were obtained. These plasma samples were used in the following cell-based ELISA.

Rep5.1 cells were cultured in DMEM-10 containing 500 μg/ml of G418. The Rep5.1 cells in log phase were suspended by trypsin treatment, and 5000 cells were distributed to each well of a 96-well pate. After 4 hours for which the cells were attached to the plate, the serially diluted drug was added to each well. After 72 hours, the cells were fixed by methanol/acetone (50/50, v/v), and the decrease of HCV proteins was measured by using HCV antibody obtained as above. The extent of decrease was estimated in percentage based on the control containing no drug, and the concentration of drug at which 50% of proteins was decreased was determined to be IC50.

The IC50 values are classified according to the following standard and the results are represented in the following Tables 1 and 2.

A: 1 μM or less

B: more than 1 μM & 10 μM or less

C: more than 10 μM & 20 μM or less

D: more than 20 μM

TABLE 1

| Level | COM. NO. |
|---|---|
| A | 6, 10, 14, 19, 22, 29, 30, 33, 35, 39, 41, 42, 43, 45, 47, 49, 50, 51, |
| | 52, 54, 56, 58, 60, 61, 65, 66, 68, 70, 72, 75, 76, 78, 82, 83, 84, |
| | 85, 88, 89, 90, 91, 92, 93, 94, 95, 96, 97, 100, 101, 103, 104, |
| | 105, 106, 107, 108, 114, 115, 116, 117, 118, 121, 122, 124, 126, |
| | 128, 131, 132, 133, 134, 135, 136, 141, 143, 147, 148, 149, 150, |
| | 151, 152, 156, 157, 164, 172, 178, 175, 180, 185, 192, 193, 199, |
| | 200, 203, 204, 206, 207, 208, 209, 214, 218, 219, 220, 222, 224, |
| | 227, 230, 231, 233, 235, 239, 240, 243, 244, 247, 249, 250, 257, |
| | 258, 259, 261, 263, 266, 273, 3′, 9′, 12′, 14′, 15′, 21′, 22′, 23′, 26′, |
| | 27′, 31′, 32′, 33′, 34′, 35′, 37′, 38′, 40′, 45′, 70′, 71′, 72′, |
| | 79′, 84′, 86′, 87′, 88′, 89′, 90′, 18″, 24″, 27″, 28″, 31″, 33″, |
| | 37″, 38″, 42″, 43″, 46″, 55″, 56″, 66″, 67″, 69″, 70″, 72″, 73″, |
| | 74″, 75″, 76″, 78″, 82″, 84″, 86″, 88″, 91″, 92″, 93″, 94″, 95″, |
| | 96″, 97″, 98″, 99″, 100″ |
| B | 7, 8, 9, 15, 18, 23, 25, 26, 34, 37, 44, 53, 55, 57, 59, 67, 69, 71, |
| | 77, 79, 80, 81, 98, 99, 102, 110, 111, 119, 120, 123, 125, 127, |
| | 137, 139, 142, 153, 163, 165, 169, 174, 176, 177, 178, 183, 195, |
| | 198, 202, 205, 210, 213, 216, 217, 221, 223, 226, 237, 238, 245, |
| | 248, 251, 252, 254, 255, 256, 265, 268, 269, 270, 271, 1′, 2′, 5′, 7′, |
| | 10′, 16′, 24′, 25′, 36′, 39′, 42′, 43′, 44′, 46′, 49′, 51′, 54′, |
| | 55′, 56′, 57′, 58′, 60′, 61′, 62′, 64′, 65′, 66′, 69′, 74′, 75′, 81′, |
| | 82′, 83′, 85′, 91′, 1″, 3″, 4″, 5″, 6″, 7″, 10″, 11″, 13″, 14″, 17″, |
| | 19″, 23″, 25″, 26″, 32″, 34″, 35″, 36″, 41″, 44″, 45″, 49″, 51″, |
| | 52″, 53″, 54″, 57″, 58″, 59″, 60″, 63″, 64″, |
| | 65″, 68″, 77″, 79″, 80″, 81″, 83″, 85″, 87″, 89″, 90″, 101″ |
| C | 12, 46, 48, 64, 138, 140, 160, 184, 201, 211, 215, 228, 232, 236, |
| | 253, 264, 29′, 41′, 53′, 78′, 80′, 9″, 39″, 61″ |
| D | 11, 13, 16, 17, 24, 27, 36, 40, 63, 74, 86, 87, 109, 112, 113, 129, |
| | 130, 146, 154, 155, 158, 159, 161, 162, 170, 181, 196, 197, 225, |
| | 229, 234, 241, 242, 246, 267, 272, 274, 4′, 6′, 8′, 11′, 13′, 18′, 19′, |
| | 20′, 28′, 30′, 47′, 48′, 50′, 52′, 59′, 63′, 67′, 68′, 76′, 77′, 2″, |
| | 8″, 12″, 15″, 16″, 20″, 21″, 22″, 29″, 30″, 40″, 47″, 48″, 50″, 62″, |
| | 71″ |

TABLE 2

| COM. NO. | IC50 (μM) |
|---|---|
| 6 | 1.0 |
| 7 | 2.5 |
| 8 | 3.0 |
| 9 | 4.0 |
| 10 | 1.0 |
| 11 | >100 |
| 12 | 22 |
| 13 | >100 |
| 14 | 0.2 |
| 15 | 3.0 |
| 16 | 74 |
| 17 | 100 |
| 18 | 5.0 |
| 19 | 1.6 |
| 22 | 0.4 |
| 23 | 5.4 |
| 24 | 70 |
| 25 | 2.0 |
| 26 | 3.0 |
| 27 | 47 |
| 29 | 0.3 |
| 30 | 0.1 |
| 33 | <0.5 |
| 34 | 2.0 |
| 35 | <0.5 |
| 36 | 84 |
| 37 | 4.0 |
| 39 | 0.5 |
| 40 | 92 |
| 41 | <0.4 |
| 42 | 0.5 |
| 43 | <0.4 |
| 44 | 3.0 |
| 45 | <0.4 |
| 46 | 30 |
| 47 | <0.5 |
| 48 | 30 |
| 49 | <0.5 |
| 50 | <0.5 |
| 51 | <0.5 |
| 52 | 0.7 |
| 53 | 6.6 |
| 54 | <0.5 |
| 55 | 2.5 |
| 56 | <0.5 |
| 57 | 3.5 |
| 58 | <0.5 |
| 59 | 3.0 |
| 60 | <0.5 |
| 61 | 0.8 |
| 63 | >100 |
| 64 | 15 |
| 65 | 1.0 |
| 66 | 0.5 |
| 67 | 6.0 |
| 68 | 0.7 |
| 69 | 1.2 |
| 70 | <0.5 |
| 71 | 2.5 |
| 72 | <0.5 |
| 74 | >100 |
| 75 | <0.5 |
| 76 | 0.6 |
| 77 | 2.3 |
| 78 | 1.0 |
| 79 | 1.3 |
| 80 | 2.3 |
| 81 | 3.0 |
| 82 | 0.3 |
| 83 | 1.0 |
| 84 | <0.4 |
| 85 | 0.1 |
| 86 | >100 |
| 87 | 56 |
| 88 | 0.7 |
| 89 | 0.8 |
| 90 | 0.4 |
| 91 | <0.4 |
| 92 | 1.0 |
| 93 | 0.7 |
| 94 | <0.4 |
| 95 | 0.7 |
| 96 | <0.4 |
| 97 | <0.4 |
| 98 | 7.0 |
| 99 | 1.8 |
| 100 | <0.4 |
| 101 | <0.4 |
| 102 | 2.0 |
| 103 | <0.4 |
| 104 | <0.4 |
| 105 | <0.4 |
| 106 | <0.4 |
| 107 | <0.4 |
| 108 | <0.4 |
| 109 | 100 |
| 110 | 17 |
| 111 | 2 |
| 112 | >100 |
| 113 | >100 |
| 114 | <0.4 |
| 115 | 0.7 |
| 116 | 0.5 |
| 117 | 0.4 |
| 118 | <0.4 |
| 119 | 1.3 |
| 120 | 1.0 |
| 121 | 0.5 |
| 122 | 0.8 |
| 123 | 8.0 |
| 124 | 0.8 |
| 125 | 2.0 |
| 126 | <0.4 |
| 127 | 3.5 |
| 128 | 1.0 |
| 129 | >100 |
| 130 | 35 |
| 131 | <0.4 |
| 132 | 0.4 |
| 133 | <0.4 |
| 134 | 0.6 |
| 135 | 0.9 |
| 136 | 0.6 |
| 137 | 10 |
| 138 | 20 |
| 139 | 7.0 |
| 140 | 13 |
| 141 | 0.7 |
| 142 | 6.0 |
| 143 | 0.4 |
| 146 | >100 |
| 147 | <0.4 |
| 148 | <0.4 |
| 149 | <0.4 |
| 150 | 1.0 |
| 151 | <0.4 |
| 152 | 0.5 |
| 153 | 2.7 |
| 154 | >100 |
| 155 | >100 |
| 156 | 0.5 |
| 157 | 1.0 |
| 158 | >100 |
| 159 | >100 |
| 160 | 12 |
| 161 | >100 |
| 162 | >100 |
| 163 | 2.5 |
| 164 | <0.4 |
| 165 | 1.4 |
| 169 | 9.0 |
| 170 | 90 |
| 172 | <0.4 |
| 173 | <0.4 |
| 174 | 7.0 |
| 175 | <0.4 |
| 176 | 2.7 |
| 177 | 2.5 |
| 178 | 1.5 |
| 179 | 3.5 |
| 180 | 0.8 |
| 181 | >100 |
| 183 | 2.5 |
| 184 | 19 |
| 185 | 0.4 |
| 192 | <0.4 |
| 193 | 1.0 |
| 195 | 2.0 |
| 196 | 43 |
| 197 | >100 |
| 198 | 10 |
| 199 | <0.4 |
| 200 | <0.4 |
| 201 | 13 |
| 202 | 9 |
| 203 | <0.4 |
| 204 | <0.4 |
| 205 | 1.8 |
| 206 | 0.6 |
| 207 | 0.3 |
| 208 | <0.4 |
| 209 | <0.4 |
| 210 | 3.0 |
| 211 | 16 |
| 213 | 4.0 |
| 214 | <0.4 |
| 215 | 11 |
| 216 | 5.0 |
| 217 | 7.0 |
| 218 | <0.4 |
| 219 | <0.4 |
| 220 | 0.8 |
| 221 | 2.5 |
| 222 | <0.4 |
| 223 | 6.0 |
| 224 | 0.7 |
| 225 | >100 |
| 226 | 2.2 |
| 227 | <0.4 |
| 228 | 23 |
| 229 | >100 |
| 230 | <0.4 |
| 231 | 0.7 |
| 232 | 20 |
| 233 | 0.2 |
| 234 | 100 |
| 235 | 0.9 |
| 236 | 14 |
| 237 | 1.7 |
| 238 | 3.0 |
| 239 | <0.4 |
| 240 | <0.4 |
| 241 | 97 |
| 242 | 63 |
| 243 | <0.4 |
| 244 | 0.5 |
| 245 | 2.7 |
| 246 | 68 |
| 247 | <0.4 |
| 248 | 5.0 |
| 249 | <0.4 |
| 250 | 0.4 |
| 251 | 1.6 |
| 252 | 1.4 |
| 253 | 13 |
| 254 | 3.0 |
| 255 | 2.0 |
| 256 | 3.4 |
| 257 | <0.4 |
| 258 | <0.4 |
| 259 | <0.4 |
| 261 | 0.8 |
| 263 | 1.7 |
| 264 | 13 |
| 265 | 6.0 |
| 266 | 0.6 |
| 267 | 24 |
| 268 | 1.0 |
| 269 | 5.0 |
| 270 | 5.0 |
| 271 | 9.0 |
| 272 | 34 |
| 273 | 0.8 |
| 274 | >100 |
| 1′ | 1.8 |
| 2′ | 2.4 |
| 3′ | 0.7 |
| 4′ | 51 |
| 5′ | 2.5 |
| 6′ | >100 |
| 7′ | 8 |
| 8′ | 21 |
| 9′ | <0.4 |
| 10′ | 1.2 |
| 11′ | 77 |
| 12′ | <0.4 |
| 13′ | 79 |
| 14′ | 0.7 |
| 15′ | 1 |
| 16′ | 2 |
| 18′ | >100 |
| 19′ | >100 |
| 20′ | 27 |
| 21′ | 0.4 |
| 22′ | <0.4 |
| 23′ | 0.7 |
| 24′ | 4.4 |
| 25′ | 4.9 |
| 26′ | 0.9 |
| 27′ | <0.4 |
| 28′ | 72 |
| 29′ | 12 |
| 30′ | >100 |
| 31′ | 0.4 |
| 32′ | 0.9 |
| 33′ | 1 |
| 34′ | 0.7 |
| 35′ | 0.8 |
| 36′ | 1.7 |
| 37′ | 0.6 |
| 38′ | 0.7 |
| 39′ | 2.6 |
| 40′ | 0.6 |
| 41′ | 15 |
| 42′ | 4.1 |
| 43′ | 1.8 |
| 44′ | 2.8 |
| 45′ | 0.8 |
| 46′ | 6 |
| 47′ | 100 |
| 48′ | 21 |
| 49′ | 8 |
| 50′ | >100 |
| 51′ | 5.4 |
| 52′ | 22 |
| 53′ | 18 |
| 54′ | 5 |
| 55′ | 1.6 |
| 56′ | 2.4 |
| 57′ | 10 |
| 58′ | 8 |
| 59′ | 34 |
| 60′ | 4 |
| 61′ | 3 |
| 62′ | 2 |
| 63′ | >100 |
| 64′ | 3 |
| 65′ | 1.4 |
| 66′ | 8 |
| 67′ | >100 |
| 68′ | >100 |
| 69′ | 3 |
| 70′ | 1 |
| 71′ | 0.6 |
| 72′ | <0.4 |
| 74′ | 7 |
| 75′ | 2.6 |
| 76′ | 62 |
| 77′ | 39 |
| 78′ | 11 |
| 79′ | 1 |
| 80′ | 11 |
| 81′ | 2.4 |
| 82′ | 2.5 |
| 83′ | 5.7 |
| 84′ | 0.9 |
| 85′ | 2.7 |
| 86′ | <0.4 |
| 87′ | <0.4 |
| 88′ | 0.4 |
| 89′ | 0.4 |
| 90′ | 0.8 |
| 91′ | 1.6 |
| 1″ | 1.6 |
| 2″ | 35 |
| 3″ | 9 |
| 4″ | 2.5 |
| 5″ | 6 |
| 6″ | 1.9 |
| 7″ | 7 |
| 8″ | >100 |
| 9″ | 20 |
| 10″ | 7 |
| 11″ | 6 |
| 12″ | 31 |
| 13″ | 5.3 |
| 14″ | 2.6 |
| 15″ | 55 |
| 16″ | >100 |
| 17″ | 9 |
| 18″ | <0.4 |
| 19″ | 1.4 |
| 20″ | 33.3 |
| 21″ | >100 |
| 22″ | 43 |
| 23″ | 9 |
| 24″ | 0.8 |
| 25″ | 2 |
| 26″ | 2 |
| 27″ | 0.4 |
| 28″ | 1 |
| 29″ | >100 |
| 30″ | 50 |
| 31″ | 1 |
| 32″ | 6 |
| 33″ | <0.4 |
| 34″ | 2.6 |
| 35″ | 1.6 |
| 36″ | 3 |
| 37″ | 0.8 |
| 38″ | <0.4 |
| 39″ | 20 |
| 40″ | >100 |
| 41″ | 7.2 |
| 42″ | <0.4 |
| 43″ | <0.4 |
| 44″ | 5 |
| 45″ | 2.7 |
| 46″ | <0.4 |
| 47″ | >100 |
| 48″ | 43 |
| 49″ | 3 |
| 50″ | 68 |
| 51″ | 2.3 |
| 52″ | 10 |
| 53″ | 7 |
| 54″ | 8 |
| 55″ | 0.5 |
| 56″ | 0.6 |
| 57″ | 6.4 |
| 58″ | 1.8 |
| 59″ | 2.5 |
| 60″ | 2.5 |
| 61″ | 16 |
| 62″ | >100 |
| 63″ | 1.6 |
| 64″ | 5.5 |
| 65″ | 1.4 |
| 66″ | 0.7 |
| 67″ | <0.4 |
| 68″ | 2.7 |
| 69″ | 0.8 |
| 70″ | 0.4 |
| 71″ | >100 |
| 72″ | 0.4 |
| 73″ | 0.4 |
| 74″ | <0.4 |
| 75″ | <0.4 |
| 76″ | 0.9 |
| 77″ | 5.6 |
| 78″ | 0.8 |
| 79″ | 6.2 |
| 80″ | 1.1 |
| 81″ | 6.5 |
| 82″ | 0.8 |
| 83″ | 6.1 |
| 84″ | 0.9 |
| 85″ | 6.5 |
| 86″ | 0.7 |
| 87″ | 6.3 |
| 88″ | 0.9 |
| 89″ | 6.8 |
| 90″ | 6.9 |
| 91″ | 0.7 |
| 92″ | 0.4 |
| 93″ | 0.5 |
| 94″ | 0.8 |
| 95″ | 0.6 |
| 96″ | 0.9 |
| 97″ | 0.6 |
| 98″ | 1.0 |
| 99″ | 1.0 |
| 100″ | 0.9 |
| 101″ | 1.2 |

1. A compound of the following formula (1): 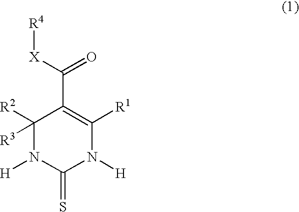
in which
X is O or S,
R1 is hydrogen,
alkyl unsubstituted or substituted by 1 to 3 substituents selected from a group consisting of halogen; alkoxy; alkoxycarbonyl; hydroxy; carboxy; and aryl,
alkoxy,
alkoxycarbonyl unsubstituted or substituted by aryl, or
aryl,
R2 and R3 each are
hydrogen,
alkyl substituted by 1 to 3 substituents selected from a group consisting of halogen; hydroxy; alkoxy; alkylsulfonyl; and aralkyloxy,
alkoxycarbonyl,
alkylsulfonylalkyl,
aryl, or
heteroaryl, or
R2 and R3 together form cycloalkyl with the carbon atom to which they are attached, or when one of R2 and R3 is hydrogen, the other is a structure selected from the following: 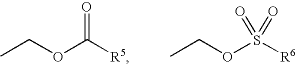
where
R5 is alkyl unsubstituted or substituted by amino,
alkylamino unsubstituted or substituted by alkylcarbonylamino; alkylcarbonyloxy; or dialkylamino,
alkoxy unsubstituted or substituted by 1 to 3 substituents selected from a group consisting of benzoyloxy; alkoxycarbonyl; alkyl; alkylsulfonyl; alkylcarbonylthio; alkoxycarbonylamino; and heterocycle which is unsubstituted or substituted by 1 to 3 substituents selected from a group consisting of alkyl and oxo, and which may include a double bond,
aryl unsubstituted or substituted by carboxy, or
aralkyl unsubstituted or substituted by alkylcarbonyloxy,
R6 is alkyl,
R4 is alkyl unsubstituted or substituted by 1 to 3 substituents selected from a group consisting of alkoxy unsubstituted or substituted by heteroaryl; carboxy; alkylthio; alkoxycarbonyl; alkylaminocarbonyl; biphenyl; aryl; aryloxy; aralkyloxy; alkylaryl; amino unsubstituted or substituted by a substituent(s) selected from a group consisting of alkyl and alkylaryl; cycloalkyl; cycloalkyloxy; alkylarylsulfonyl; heteroaryl unsubstituted or substituted by halogen; and heteroarylthio,
alkenyl unsubstituted or substituted by 1 to 3 substituents selected from a group consisting of halogen; aryl; and heteroaryl,
alkinyl unsubstituted or substituted by 1 to 3 substituents selected from a group consisting of alkoxy; alkenyloxy; alkinyloxy; aralkyloxy; aryl; and heteroaryl,
—(CH2)N-cycloalkyl,
—(CH2)N-cycloalkyl substituted by aryl which is unsubstituted or substituted by alkoxy or halogen; or fused with heteroaryl unsubstituted or substituted by halogen,
—(CH2)N-cycloalkenyl,
—(CH2)N-cycloalkenyl substituted by 1 to 3 substituents selected from a group consisting of alkyl and alkenyl,
—(CH2)N-aryl,
—(CH2)N-aryl substituted by 1 to 5 substituents selected from a group consisting of nitro; cyano; hydroxy; halogen; alkyl; halogenoalkyl; alkoxy; halogenoalkoxy; alkylthio; halogenoalkylthio; alkylsulfonyl; alkoxycarbonyl; alkoxycarbonyloxy; amino unsubstituted or substituted by a substituent(s) selected from a group consisting of alkyl, alkoxyalkyl, alkenyl, cycloalkyl, and cycloalkylalkyl; aryloxy; aralkyloxy unsubstituted or substituted by alkoxy; and heterocycle,
—(CH2)N-heterocycle,
—(CH2)N-heterocycle substituted by 1 to 3 substituents selected from a group consisting of oxo; nitro; alkyl; aralkyl; and aryl unsubstituted or substituted by nitro,
—(CH2)N-heteroaryl, or
—(CH2)N-heteroaryl substituted by 1 to 3 substituents selected from a group consisting of oxo; nitro; halogen; alkyl; alkoxy; alkylthio; alkylarylsulfonyl; aryl; and heteroaryl unsubstituted or substituted by 1 to 3 substituents selected from a group consisting of alkyl and halogenoalkyl,
wherein N is an integer of 0 to 10, and when N is not 0, the —CH2— group is unsubstituted or substituted by halogen, or its pharmaceutically acceptable salt, hydrate, solvate, or isomer including tautomer.

2. The compound of claim 1 wherein
X is O or S,
R1 is hydrogen or
alkyl unsubstituted or substituted by 1 to 3 substituents selected from a group consisting of halogen; alkoxy; alkoxycarbonyl; carboxy; and aryl,
R2 and R3 each are
hydrogen or
alkyl substituted by 1 to 3 substituents selected from a group consisting of halogen; hydroxy; alkoxy; and aralkyloxy,
R4 is alkyl unsubstituted or substituted by 1 to 3 substituents selected from a group consisting of alkoxy; alkoxycarbonyl; biphenyl; aryl; aryloxy; aralkyloxy; alkylaryl; amino unsubstituted or substituted by a substituent(s) selected from a group consisting of alkyl and alkylaryl; cycloalkyl; cycloalkyloxy; alkylarylsulfonyl; heteroaryl; and heteroarylthio,
alkenyl unsubstituted or substituted by 1 to 3 substituents selected from a group consisting of halogen; aryl; and heteroaryl,
alkinyl unsubstituted or substituted by 1 to 3 substituents selected from a group consisting of alkoxy; alkenyloxy; alkinyloxy; aralkyloxy; aryl; and heteroaryl,
—(CH2)N-cycloalkyl,
—(CH2)N-cycloalkenyl substituted by 1 to 3 substituents selected from a group consisting of alkyl and alkenyl,
—(CH2)N-aryl,
—(CH2)N-aryl substituted by 1 to 3 substituents selected from a group consisting of nitro; cyano; hydroxy; halogen; alkyl; halogenoalkyl; alkoxy; halogenoalkoxy; alkylthio; halogenoalkylthio; alkylsulfonyl; alkoxycarbonyl; alkoxycarbonyloxy; amino unsubstituted or substituted by a substituent(s) selected from a group consisting of alkyl and alkoxyalkyl; and aralkyloxy unsubstituted or substituted by alkoxy,
—(CH2)N-heterocycle,
—(CH2)N-heterocycle substituted by 1 to 3 substituents selected from a group consisting of oxo; nitro; alkyl; aralkyl; and aryl unsubstituted or substituted by nitro,
—(CH2)N-heteroaryl, or
—(CH2)N-heteroaryl substituted by 1 to 3 substituents selected from a group consisting of oxo; halogen; alkyl; alkylthio; alkylarylsulfonyl; aryl; and heteroaryl unsubstituted or substituted by 1 to 3 substituents selected from a group consisting of alkyl and halogenoalkyl,
wherein N is an integer of 0 to 10, its pharmaceutically acceptable salt, hydrate, solvate, or isomer including tautomer.

3. The compound of claim 1 or 2 wherein R1 is hydrogen or unsubstituted alkyl, its pharmaceutically acceptable salt, hydrate, solvate, or isomer including tautomer.

4. The compound of claim 1 or 2 wherein R2 is hydrogen, its pharmaceutically acceptable salt, hydrate, solvate, or isomer including tautomer.

5. The compound of claim 1 or 2 wherein R3 is hydrogen, or alkyl substituted by halogen or hydroxy, its pharmaceutically acceptable salt, hydrate, solvate, or isomer including tautomer.

6. The compound of claim 1 wherein R3 is hydrogen, hydroxymethyl, or fluoromethyl, its pharmaceutically acceptable salt, hydrate, solvate, or isomer including tautomer.

7. The compound of claim 1 or 2 wherein X is O or S, R1 is unsubstituted alkyl, R2 is hydrogen, and R3 is hydrogen, hydroxymethyl, or fluoromethyl, its pharmaceutically acceptable salt, hydrate, solvate, or isomer including tautomer.

8. A process for preparing the compound of formula (1) as defined in claim 1, its pharmaceutically acceptable salt, hydrate, solvate, or isomer including tautomer characterized in that
(a) an oxazinane of the following formula (2): 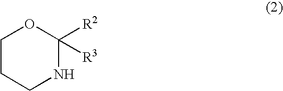
in which R2 and R3 are defined as claim 1, an acetoacetate of the following formula (3): 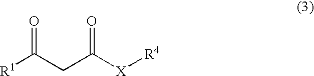
in which X, R1, and R4 are defined as claim 1, and a thiourea of the following formula (4a): 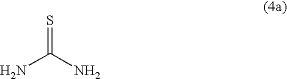
are reacted, or
(b) a carbonyl compound of the following formula (5): 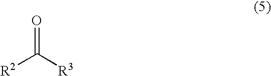
in which R2 and R3 are defined as claim 1, the acetoacetate of formula (3), and an isothiourea hydrochloride of the following formula (4b): 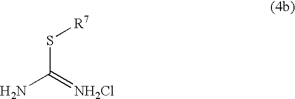
in which R7 is aralkyl or alkoxycarbonyl, each of which is unsubstituted or substituted by alkoxy, are reacted.

9. A composition for the treatment or prevention of hepatitis C, comprising as an active ingredient the compound of formula (1) as defined in claim 1, its pharmaceutically acceptable salt, hydrate, solvate, or isomer including tautomer together with pharmaceutically acceptable carrier.

10. A process for preparing the composition for the treatment or prevention of hepatitis C as defined in claim 9, comprising admixing the compound of formula (1) as defined in claim 1, its pharmaceutically acceptable salt, hydrate, solvate, or isomer including tautomer with pharmaceutically acceptable carrier.

11. A method for the treatment or prevention of hepatitis C, comprising administering the compound of formula (1) as defined in claim 1, its pharmaceutically acceptable salt, hydrate, solvate, or isomer including tautomer to a patient suffering from hepatitis C.

12. A use of the compound of formula (1) as defined in claim 1, its pharmaceutically acceptable salt, hydrate, solvate, or isomer including tautomer for the treatment or prevention of hepatitis C.
